SEQUENCE LISTING (314 sequences)

SEQ ID NO: 1 - protein, 12 aa, synthetic construct
  REGION 1..12: note = Synthetic
QSLLNSGNQKNY

SEQ ID NO: 2 - skipped

SEQ ID NO: 3 - protein, 9 aa, synthetic construct
  REGION 1..9: note = Synthetic
QNGYYFPFT

SEQ ID NO: 4 - protein, 8 aa, synthetic construct
  REGION 1..8: note = Synthetic
GYTFTGYI

SEQ ID NO: 5 - protein, 8 aa, synthetic construct
  REGION 1..8: note = Synthetic
INPYNDGT

SEQ ID NO: 6 - protein, 11 aa, synthetic construct
  REGION 1..11: note = Synthetic
ARAYFGNSFAY

SEQ ID NO: 7 - skipped

SEQ ID NO: 8 - protein, 9 aa, synthetic construct
  REGION 1..9: note = Synthetic
QNDYIYPYT

SEQ ID NO: 9 - protein, 8 aa, synthetic construct
  REGION 1..8: note = Synthetic
GYTFTTYP

SEQ ID NO: 10 - protein, 8 aa, synthetic construct
  REGION 1..8: note = Synthetic
FHPYNDDT

SEQ ID NO: 11 - protein, 13 aa, synthetic construct
  REGION 1..13: note = Synthetic
ARRAYGYPYAMDY

SEQ ID NO: 12 - protein, 12 aa, synthetic construct
  REGION 1..12: note = Synthetic
QSLLNSGNRKNY

SEQ ID NO: 13 - protein, 9 aa, synthetic construct
  REGION 1..9: note = Synthetic
QNAYSYPFT

SEQ ID NO: 14 - protein, 8 aa, synthetic construct
  REGION 1..8: note = Synthetic
GFTFSTFG

SEQ ID NO: 15 - protein, 8 aa, synthetic construct
  REGION 1..8: note = Synthetic
ITSGNSPI

SEQ ID NO: 16 - protein, 12 aa, synthetic construct
  REGION 1..12: note = Synthetic
ARSSYYGNSMDY

SEQ ID NO: 17 - protein, 12 aa, synthetic construct
  REGION 1..12: note = Synthetic
QSLLNAGNQKNY

SEQ ID NO: 18 - protein, 12 aa, synthetic construct
  REGION 1..12: note = Synthetic
QSLLESGNQKNY

SEQ ID NO: 19 - protein, 9 aa, synthetic construct
  REGION 1..9: note = Synthetic
QNAYYFPFT

SEQ ID NO: 20 - protein, 9 aa, synthetic construct
  REGION 1..9: note = Synthetic
QEGYYFPFT

SEQ ID NO: 21 - protein, 8 aa, synthetic construct
  REGION 1..8: note = Synthetic
INPYNDDT

SEQ ID NO: 22 - protein, 11 aa, synthetic construct
  REGION 1..11: note = Synthetic
ARAYFGNAFAY

SEQ ID NO: 23 - protein, 12 aa, synthetic construct
  REGION 1..12: note = Synthetic
QSLLESGNRKNY

SEQ ID NO: 24 - protein, 12 aa, synthetic construct
  REGION 1..12: note = Synthetic
QSLLNAGNRKNY

SEQ ID NO: 25 - protein, 8 aa, synthetic construct
  REGION 1..8: note = Synthetic
ITSGQSPI

SEQ ID NO: 26 - protein, 8 aa, synthetic construct
  REGION 1..8: note = Synthetic
ITSGESPI

SEQ ID NO: 27 - protein, 12 aa, synthetic construct
  REGION 1..12: note = Synthetic
ARSSYYGQSMDY

SEQ ID NO: 28 - protein, 12 aa, synthetic construct
  REGION 1..12: note = Synthetic
ARSSYYGESMDY

SEQ ID NO: 29 - protein, 12 aa, synthetic construct
  REGION 1..12: note = Synthetic
ARSSYYGNAMDY

SEQ ID NO: 30 - protein, 261 aa, synthetic construct
  REGION 1..261: note = Synthetic
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 31 - protein, 12 aa, synthetic construct
  REGION 1..12: note = Synthetic
QSLLNSGNQRNY

SEQ ID NO: 32 - protein, 12 aa, synthetic construct
  REGION 1..12: note = Synthetic
QSLLNSGNLKNY

SEQ ID NO: 33 - protein, 12 aa, synthetic construct
  REGION 1..12: note = Synthetic
QSLLNGGNQKNY

SEQ ID NO: 34 - protein, 12 aa, synthetic construct
  REGION 1..12: note = Synthetic
QSLLNSGNQKKY

SEQ ID NO: 35 - protein, 12 aa, synthetic construct
  REGION 1..12: note = Synthetic
QSLFNSGNQRNY

SEQ ID NO: 36 - protein, 12 aa, synthetic construct
  REGION 1..12: note = Synthetic
MSLFNSGNQKSY

SEQ ID NO: 37 - protein, 12 aa, synthetic construct
  REGION 1..12: note = Synthetic
QSLFNSGNQKNY

SEQ ID NO: 38 - protein, 12 aa, synthetic construct
  REGION 1..12: note = Synthetic
QSVFNSGNQKNY

SEQ ID NO: 39-41 - skipped (3 entries)

SEQ ID NO: 42 - protein, 9 aa, synthetic construct
  REGION 1..9: note = Synthetic
QNDYFYPFT

SEQ ID NO: 43 - protein, 9 aa, synthetic construct
  REGION 1..9: note = Synthetic
QNVYIYPFT

SEQ ID NO: 44 - protein, 9 aa, synthetic construct
  REGION 1..9: note = Synthetic
QNDYYFPFT

SEQ ID NO: 45 - protein, 9 aa, synthetic construct
  REGION 1..9: note = Synthetic
QNDLYYPWT

SEQ ID NO: 46 - protein, 9 aa, synthetic construct
  REGION 1..9: note = Synthetic
QNGYSYPFT

SEQ ID NO: 47 - protein, 9 aa, synthetic construct
  REGION 1..9: note = Synthetic
QNNYYFPLT

SEQ ID NO: 48 - protein, 9 aa, synthetic construct
  REGION 1..9: note = Synthetic
QNVYSYPLT

SEQ ID NO: 49 - protein, 9 aa, synthetic construct
  REGION 1..9: note = Synthetic
QNSYSYPFT

SEQ ID NO: 50 - protein, 9 aa, synthetic construct
  REGION 1..9: note = Synthetic
QNNFIYPLT

SEQ ID NO: 51 - protein, 9 aa, synthetic construct
  REGION 1..9: note = Synthetic
HNDYIYPLT

SEQ ID NO: 52 - protein, 9 aa, synthetic construct
  REGION 1..9: note = Synthetic
QNNYYYPFT

SEQ ID NO: 53 - protein, 9 aa, synthetic construct
  REGION 1..9: note = Synthetic
QNAYYYPLT

SEQ ID NO: 54 - protein, 9 aa, synthetic construct
  REGION 1..9: note = Synthetic
QNAYFYPCT

SEQ ID NO: 55 - protein, 9 aa, synthetic construct
  REGION 1..9: note = Synthetic
QNDYYFPLT

SEQ ID NO: 56 - protein, 9 aa, synthetic construct
  REGION 1..9: note = Synthetic
QNAYFYPFT

SEQ ID NO: 57 - protein, 9 aa, synthetic construct
  REGION 1..9: note = Synthetic
QNNYFYPLT

SEQ ID NO: 58 - protein, 9 aa, synthetic construct
  REGION 1..9: note = Synthetic
QNNYIYPLA

SEQ ID NO: 59 - protein, 8 aa, synthetic construct
  REGION 1..8: note = Synthetic
GYAFTNYL

SEQ ID NO: 60 - protein, 8 aa, synthetic construct
  REGION 1..8: note = Synthetic
GFSLTSYG

SEQ ID NO: 61 - protein, 8 aa, synthetic construct
  REGION 1..8: note = Synthetic
GFTFNSFG

SEQ ID NO: 62 - protein, 8 aa, synthetic construct
  REGION 1..8: note = Synthetic
GFTFNTNA

SEQ ID NO: 63 - protein, 8 aa, synthetic construct
  REGION 1..8: note = Synthetic
GYTFPTYS

SEQ ID NO: 64 - protein, 8 aa, synthetic construct
  REGION 1..8: note = Synthetic
GFTFSNYG

SEQ ID NO: 65 - protein, 8 aa, synthetic construct
  REGION 1..8: note = Synthetic
GYTFTNYG

SEQ ID NO: 66 - protein, 8 aa, synthetic construct
  REGION 1..8: note = Synthetic
GYTFTKYG

SEQ ID NO: 67 - protein, 8 aa, synthetic construct
  REGION 1..8: note = Synthetic
GFSLSSYG

SEQ ID NO: 68 - protein, 8 aa, synthetic construct
  REGION 1..8: note = Synthetic
GLSLTSFG

SEQ ID NO: 69 - protein, 8 aa, synthetic construct
  REGION 1..8: note = Synthetic
GFSLISYG

SEQ ID NO: 70 - protein, 8 aa, synthetic construct
  REGION 1..8: note = Synthetic
GFSLNTYG

SEQ ID NO: 71 - protein, 8 aa, synthetic construct
  REGION 1..8: note = Synthetic
GFSLINYG

SEQ ID NO: 72 - protein, 8 aa, synthetic construct
  REGION 1..8: note = Synthetic
GYTFTGFL

SEQ ID NO: 73 - protein, 8 aa, synthetic construct
  REGION 1..8: note = Synthetic
DFSLTKYG

SEQ ID NO: 74 - protein, 9 aa, synthetic construct
  REGION 1..9: note = Synthetic
GYSITSGYF

SEQ ID NO: 75 - protein, 8 aa, synthetic construct
  REGION 1..8: note = Synthetic
GFSLSNYG

SEQ ID NO: 76 - protein, 8 aa, synthetic construct
  REGION 1..8: note = Synthetic
GYSFTNFL

SEQ ID NO: 77 - protein, 8 aa, synthetic construct
  REGION 1..8: note = Synthetic
INPGNGGS

SEQ ID NO: 78 - protein, 7 aa, synthetic construct
  REGION 1..7: note = Synthetic
IWGDGNT

SEQ ID NO: 79 - protein, 8 aa, synthetic construct
  REGION 1..8: note = Synthetic
ISGGSNTI

SEQ ID NO: 80 - protein, 10 aa, synthetic construct
  REGION 1..10: note = Synthetic
IRSKSNNYAT

SEQ ID NO: 81 - protein, 9 aa, synthetic construct
  REGION 1..9: note = Synthetic
INPGNGGSN

SEQ ID NO: 82 - protein, 8 aa, synthetic construct
  REGION 1..8: note = Synthetic
INPSTIYT

SEQ ID NO: 83 - protein, 8 aa, synthetic construct
  REGION 1..8: note = Synthetic
FSYGDSHN

SEQ ID NO: 84 - protein, 8 aa, synthetic construct
  REGION 1..8: note = Synthetic
INANTGEP

SEQ ID NO: 85 - protein, 8 aa, synthetic construct
  REGION 1..8: note = Synthetic
ISTNTGEP

SEQ ID NO: 86 - protein, 8 aa, synthetic construct
  REGION 1..8: note = Synthetic
INPGRSGT

SEQ ID NO: 87 - protein, 7 aa, synthetic construct
  REGION 1..7: note = Synthetic
IWAGGST

SEQ ID NO: 88 - protein, 7 aa, synthetic construct
  REGION 1..7: note = Synthetic
IWAGGRT

SEQ ID NO: 89 - protein, 7 aa, synthetic construct
  REGION 1..7: note = Synthetic
MLSDGNT

SEQ ID NO: 90 - protein, 7 aa, synthetic construct
  REGION 1..7: note = Synthetic
IRGDGNT

SEQ ID NO: 91 - protein, 7 aa, synthetic construct
  REGION 1..7: note = Synthetic
IWTGGNT

SEQ ID NO: 92 - protein, 7 aa, synthetic construct
  REGION 1..7: note = Synthetic
ISYDGSN

SEQ ID NO: 93 - protein, 7 aa, synthetic construct
  REGION 1..7: note = Synthetic
IWAGGNT

SEQ ID NO: 94 - protein, 8 aa, synthetic construct
  REGION 1..8: note = Synthetic
INPTNGRT

SEQ ID NO: 95 - protein, 8 aa, synthetic construct
  REGION 1..8: note = Synthetic
INPNTIYT

SEQ ID NO: 96 - protein, 11 aa, synthetic construct
  REGION 1..11: note = Synthetic
ARIYYGNSFAY

SEQ ID NO: 97 - protein, 12 aa, synthetic construct
  REGION 1..12: note = Synthetic
AKQGLYGHAMDY

SEQ ID NO: 98 - protein, 11 aa, synthetic construct
  REGION 1..11: note = Synthetic
TRLALGNAMDY

SEQ ID NO: 99 - protein, 14 aa, synthetic construct
  REGION 1..14: note = Synthetic
VSGAYYGNSKAFDY

SEQ ID NO: 100 - protein, 13 aa, synthetic construct
  REGION 1..13: note = Synthetic
AREGYGRGNAMDY

SEQ ID NO: 101 - protein, 11 aa, synthetic construct
  REGION 1..11: note = Synthetic
ARFGRGNTMDY

SEQ ID NO: 102 - protein, 11 aa, synthetic construct
  REGION 1..11: note = Synthetic
ARLTRGNSFDY

SEQ ID NO: 103 - protein, 11 aa, synthetic construct
  REGION 1..11: note = Synthetic
ARLVRGNSFDF

SEQ ID NO: 104 - protein, 12 aa, synthetic construct
  REGION 1..12: note = Synthetic
ARTRYGGNAMDY

SEQ ID NO: 105 - protein, 11 aa, synthetic construct
  REGION 1..11: note = Synthetic
ARSLYGNSLDS

SEQ ID NO: 106 - protein, 11 aa, synthetic construct
  REGION 1..11: note = Synthetic
ARSLYGNSFDY

SEQ ID NO: 107 - protein, 12 aa, synthetic construct
  REGION 1..12: note = Synthetic
ARDRYGGNSLDY

SEQ ID NO: 108 - protein, 12 aa, synthetic construct
  REGION 1..12: note = Synthetic
ARHKAYGNAMDY

SEQ ID NO: 109 - protein, 11 aa, synthetic construct
  REGION 1..11: note = Synthetic
AKVGRGNAMDH

SEQ ID NO: 110 - protein, 11 aa, synthetic construct
  REGION 1..11: note = Synthetic
ARLDYGNAMDY

SEQ ID NO: 111 - protein, 12 aa, synthetic construct
  REGION 1..12: note = Synthetic
ARNGYYGNAMDY

SEQ ID NO: 112 - protein, 11 aa, synthetic construct
  REGION 1..11: note = Synthetic
GRLDYGNAMDY

SEQ ID NO: 113 - protein, 8 aa, synthetic construct
  REGION 1..8: note = Synthetic
ASFRFFAY

SEQ ID NO: 114 - protein, 13 aa, synthetic construct
  REGION 1..13: note = Synthetic
ARHGYGKGNAMDN

SEQ ID NO: 115 - protein, 11 aa, synthetic construct
  REGION 1..11: note = Synthetic
ARIYYGNSMDY

SEQ ID NO: 116 - protein, 11 aa, synthetic construct
  REGION 1..11: note = Synthetic
ARSLYGNSFDH

SEQ ID NO: 117 - protein, 113 aa, synthetic construct
  REGION 1..113: note = Synthetic
DIVMTQSPSSLTVTAGEKVTMNCKSSQSLLNSGNQRNYLTWYQQKPGQPPKLLIYWASTRESGVPDRFTGSGSGTDFTLTISSVQADDLAVYYCQNDYFYPFTFGAGTNLELK

SEQ ID NO: 118 - protein, 113 aa, synthetic construct
  REGION 1..113: note = Synthetic
DIMMTQSPSSLTVTTGEKVTLTCKSSQSLLNSGNLKNYLTWYQQKPGHPPKLLIYWASTRESGVPVRFTGSGSGTDFTLTISSVQAEDLTVYYCQNVYIYPFTFGSGTKLEMR

SEQ ID NO: 119 - protein, 113 aa, synthetic construct
  REGION 1..113: note = Synthetic
DIVMTQSPSSLTVTAGEKVTMSCKSSQSLLNSGNQKNYLTWYQQKPGQPPKLLIYWASTRESGVPDRFTGSGSGTYFTLTISSVQAADLAVYYCQNDYYFPFTFGSGTKLEIK

SEQ ID NO: 120 - protein, 113 aa, synthetic construct
  REGION 1..113: note = Synthetic
DTVMTQFPSSLSVSAGEKVTMSCKSSQSLLNGGNQKNYLAWYQQKPGQPPKLLIYGASTRESGVPDRFTGSGSGTDFTLTISSVQAEDLAVYYCQNDLYYPWTFGGGTKLEFK

SEQ ID NO: 121 - protein, 113 aa, synthetic construct
  REGION 1..113: note = Synthetic
DIVMTQSPSSLTVTAGEKVIMSCKSNQSLLNSGNQKKYLTWYQQKPGQSPKLLIYWASTRESGVPDRFTGSESGTDFTLTISSVRAEDLAVYYCQNGYSYPFTFGSGTKLEMK

SEQ ID NO: 122 - protein, 113 aa, synthetic construct
  REGION 1..113: note = Synthetic
DIVMTQSPSSLTVTAGGKVTVSCKSSQSLLNSGNQKNYLTWYQQKPGQPPKLLIYWASTRESGVPDRFTGSGSGTDFTLTISSVQTEDLAVYYCQNNYYFPLTFGAGTKLELK

SEQ ID NO: 123 - protein, 113 aa, synthetic construct
  REGION 1..113: note = Synthetic
DIVMTQSPSSLTVTAGEKVTMSCKSSQSLFNSGNQRNYLTWYQQKPGQPPKLLIYWASTRESGVPDRFTGSGSGTDFILTITKVQAEDLAVYYCQNVYSYPLTFGAGTKLELK

SEQ ID NO: 124 - protein, 113 aa, synthetic construct
  REGION 1..113: note = Synthetic
DIVMTQSPSSLTVTAGEKVTMSCKSSQSLLNSGNQKNYLTWYQQRPGQPPKLLIYRASSRESGVPVRFTGSGSGTDFTLTISSVQAEDLAVYYCQNDYIYPYTFGGGTKLEMN

SEQ ID NO: 125 - protein, 113 aa, synthetic construct
  REGION 1..113: note = Synthetic
DIVMTQSPSSLTVTAGEKVTMSCKSSQSLLNSGNQKNYLTWYQQKPGQPPTLLIFWAFTRESGVPDRFTGSGSGTDFTLTINSVQAEDLAVYYCQNSYSYPFTFGSGTKLEIK

SEQ ID NO: 126 - protein, 113 aa, synthetic construct
  REGION 1..113: note = Synthetic
DIVMTQSPSSLTVTAGEKVTMSCKSSQSLLNSGNQRNYLTWYQQKPGQPPRLLIYWSSTRDSGVPDRFTGSGSRTDFTLTISSVQAEDLAVYYCQNNFIYPLTFGAGTKLELK

SEQ ID NO: 127 - protein, 113 aa, synthetic construct
  REGION 1..113: note = Synthetic
DIVMTQSPSSLTVTPGEKVTMSCRSSMSLFNSGNQKSYLSWYHQKPGQPPKLLIYWASTRDSGVPVRFTGSGSGTDFTLTISSVQAEDLAVYYCHNDYIYPLTFGAGTKLELK

SEQ ID NO: 128 - protein, 113 aa, synthetic construct
  REGION 1..113: note = Synthetic
DIVMTQSPSSLTVTAGEKVTMNCRSIQSLLNSGNQKNYLSWYQQKPGQPPKLLIYWASTRESGVPDRFTGSGSGTDFTLTIRSVLDEDLAVYYCQNSYSYPFTFGSGTKLEMK

SEQ ID NO: 129 - protein, 113 aa, synthetic construct
  REGION 1..113: note = Synthetic
DIVMTQSPSSLTVTAGEKVTLTCRSSQSLLNSGNRKNYLTWYQQIPGQPPKLLIYWASTRESGVPDRFTGSGSGTYFTLTISSVQAEDLAVYYCQNAYSYPFTFGSGTKLEKK

SEQ ID NO: 130 - protein, 113 aa, synthetic construct
  REGION 1..113: note = Synthetic
DIVMTQSPSSLTVTAGEKVTMTCKSSQSLFNSGNQRNYLTWYQRKPGQPPKLLIYWASTRESGVPDRFTGSGSGTYFTLTVSSVQAEDLAVYYCQNAYYFPFTFGSGTKLEKK

SEQ ID NO: 131 - protein, 113 aa, synthetic construct
  REGION 1..113: note = Synthetic
DIVMTQSPSSLTVTARERVSMTCKSSQSLFNSGNQRNYLTWYQQKPGQPPKLLIYWSSTRESGVPDRFTGSGSGTDFTLTISSVQAEDLAIYFCQNNYYYPFTFGSGTKLEIK

SEQ ID NO: 132 - protein, 113 aa, synthetic construct
  REGION 1..113: note = Synthetic
DIVMTQSPSSLTVTAGERVTMRCRSTQSLFNSGNQKNYLTWYQQKPGQPPKLLIYWASTRESGVPDRFTGGGSGTDFTLTISSVQAEDLAVYYCQNAYYYPLTFGAGTKLERK

SEQ ID NO: 133 - protein, 113 aa, synthetic construct
  REGION 1..113: note = Synthetic
DIVMTQSPSSLTVTAGERVTMSCKPSQSLLNSGNQKNYLAWYQQKPGQPPKLLLYWASTRESGVPDRFKGSGSGTDFTLTISSVQAEDLAVYYCQNAYFYPCTFGGGTKLEMK

SEQ ID NO: 134 - protein, 113 aa, synthetic construct
  REGION 1..113: note = Synthetic
DIVMTQSPSSLTVTAGEKVTMRCRSTQSLFNSGNQRNYLTWYQQKPGQPPKLLIYWASTRESGVPDRFTGSGSGTDFTLTISSVQAEDLAVYYCQNAYYYPLTFGVGTKLERK

SEQ ID NO: 135 - protein, 113 aa, synthetic construct
  REGION 1..113: note = Synthetic
DIVMTQSPSSLTVTAGERVTMSCKSSQSLLNSGNQKNYLAWYQQKPGQPPKLLLYWASTRESGVPDRFKGSGSGTDFTLTISSVQAEDLAVYYCQNAYFYPCTFGGGTKLEMK

SEQ ID NO: 136 - protein, 113 aa, synthetic construct
  REGION 1..113: note = Synthetic
DIVMTQSPSSLTVTTGEKVTMSCKSSQSVFNSGNQKNYLTWYQQKPGQPPKLLVYWASTRESGVPARFTGSGSGTVFTLTISSVQAEDLAVYYCQNDYYFPLTFGAGTRLELK

SEQ ID NO: 137 - protein, 113 aa, synthetic construct
  REGION 1..113: note = Synthetic
DIVMTQSPSSQTVTAGEKVTLSCRSSQSLLNGGNQKNYLTWYQQKPGQPPKLLIYWASTRESGVPDRFTGSGSGTYFTFTISSVQAEDLAVYYCQNAYFYPFTFGAGTKLELK

SEQ ID NO: 138 - protein, 113 aa, synthetic construct
  REGION 1..113: note = Synthetic
DIVMTQSPSSLTVTAGEKVTMNCKSSQSLLNSGNQRNYLSWYQQEPGQPPKLLIYWASTRESGVPDRFTGSGSGTDFTLTISNIQAEDLALYFCQNAYFYPFTFGSGTKLEIK

SEQ ID NO: 139 - protein, 113 aa, synthetic construct
  REGION 1..113: note = Synthetic
DILMTQSPSSLTVTAGEKVTMSCKSSQSLLNSGNQKNYLTWYQQKPGQSPKLLIYWASTRESGVPDRFTGSGSGTDFTLTISSVQAEDLAVYYCQNNYFYPLTFGVGTKLELK

SEQ ID NO: 140 - protein, 113 aa, synthetic construct
  REGION 1..113: note = Synthetic
DIVMTQSPSSLTVTTGEKVTMSCKSSQSLFNSGNQKNYLTWYQQKPGQPPKLLIYWASTRESGVPDRFTGSGSGTYFTLTISSVQAEDLAVYYCQNNYIYPLAFGTGTKLELK

SEQ ID NO: 141 - protein, 113 aa, synthetic construct
  REGION 1..113: note = Synthetic
DIVMTQSPSSLTVTAGEKVTMSCKSSQSLLNSGNQKNYLTWYQQRPGQPPKLLMYWASTRESGVPDRFTGSGSGTDFTLTISSGQAEDLAIYFCQNGYYFPFTFGSGTKLETK

SEQ ID NO: 142 - protein, 113 aa, synthetic construct
  REGION 1..113: note = Synthetic
DIVMTQSPSSLTVTAGEKVTMRCRSSQSLFNSGNQRNYLTWYQQKPGQPPKLLIYWASTRESGVPDRFTGSGSGTDFTLTISSVQAEDLAVYYCQNNYIYPLAFGAGTKLELK

SEQ ID NO: 143 - protein, 113 aa, synthetic construct
  REGION 1..113: note = Synthetic
DIVMTQSPSSLTVTAGEKVTMSCKSSQSLFNSGNQKNYLTWYQQKPGQPPKLLIYWASTRESGVPDRFTGSGSGTDFTLTISSVQAEDLAVYYCQNNYIYPLAFGAGTKLELK

SEQ ID NO: 144 - protein, 113 aa, synthetic construct
  REGION 1..113: note = Synthetic
DIVMTQSPSSLTVTAGEKVTMRCKSTQSLLNSGNQRNYLTWYQQKPGQSPKLLIYWASTRESGVPERFTGSGSGTDFTLTISSVQAEDLAVYYCQNAYYYPLTFGPGTKLERK

SEQ ID NO: 145 - protein, 118 aa, synthetic construct
  REGION 1..118: note = Synthetic
QVQLHQSGTELVRPGTSVKVSCKASGYAFTNYLLEWVKQRPGQGLEWIGEINPGNGGSNYNEKFKGKATLTADKSSSTAYMQLSSLTSVDSAVYFCARIYYGNSFAYWGQGTLVTVSA

SEQ ID NO: 146 - protein, 118 aa, synthetic construct
  REGION 1..118: note = Synthetic
QVQLKESGPGLVAPSQSLSITCTVSGFSLTSYGVSWVRQPPGKGLEWLGVIWGDGNTIYHSALKSRLSISRDNSKRQVFLKVNSLQIDDTATYYCAKQGLYGHAMDYWGQGTSVIVSS

SEQ ID NO: 147 - protein, 118 aa, synthetic construct
  REGION 1..118: note = Synthetic
DVQLVESGGGLVQPGGSRKLSCTASGFTFNSFGMNWVRQAPEKGLEWVAFISGGSNTIHYLDTVKGRFTISRDNPKNTLFLQMTSLRSEDTAMYYCTRLALGNAMDYWGQGTSVIVSS

SEQ ID NO: 148 - protein, 123 aa, synthetic construct
  REGION 1..123: note = Synthetic
EVQHVETGGGLVQPKGSLKLSCAASGFTFNTNAMNWVRQAPGKGLEWVARIRSKSNNYATYYADSVKDRFTISRDDSQSMLYVQMNNLKTEDTAMYYCVSGAYYGNSKAFDYWGQGTLVTVSA

SEQ ID NO: 149 - protein, 120 aa, synthetic construct
  REGION 1..120: note = Synthetic
QVQLQQSGAELARPGASVKMSCKASGYTFPTYSIHWLKQGPGQGLEWIGYINPSTIYTNYNQKFKYKATLTADKSSSTAYIQLSSLTSDDSAVYYCAREGYGRGNAMDYWGQGTSVTVSS

SEQ ID NO: 150 - protein, 118 aa, synthetic construct
  REGION 1..118: note = Synthetic
EVQLVESGGDLVKPGGSLKLSCAASGFTFSNYGMSWVRQTPDKRLEWVATFSYGDSHNYYSDSVKGRFTISRDIAKDALYLQMSSLRSEDTAIYYCARFGRGNTMDYWGQGTSVTVSL

SEQ ID NO: 151 - protein, 118 aa, synthetic construct
  REGION 1..118: note = Synthetic
QIQLVQSGPELRKPGETVKISCKASGYTFTNYGMNWVKQAPGKGLKWMAWINANTGEPTYAEEFKGRFAFSLETSARSAYLQINSLKNEDTATYFCARLTRGNSFDYWGQGTTLTVSS

SEQ ID NO: 152 - protein, 118 aa, synthetic construct
  REGION 1..118: note = Synthetic
QIQLVQSGPELKKPGETVKISCKASGYTFTKYGMNWVRQAPGKGLKWMGWISTNTGEPTYAEEFKGRFAFSLETSASTAFLQINNLKNEDTATYFCARLVRGNSFDFWGQGITLTVSS

SEQ ID NO: 153 - protein, 120 aa, synthetic construct
  REGION 1..120: note = Synthetic
QVQLQQSGGELVKPGASVKMSCKAFGYTFTTYPIEWMKQNHGKSLEWIGNFHPYNDDTKYNEKFKGKAKLTVEKSSSTVYLEVSRLTSDDSAVYYCARRAYGYPYAMDYWGQGTSVTVSS

SEQ ID NO: 154 - protein, 119 aa, synthetic construct
  REGION 1..119: note = Synthetic
QVHLQQSGAEVVRPGTSVKVSCKASGYAFTNYLIEWIKKRPGQGLEWVGVINPGRSGTNYNEKFKGKATLTADKSSSTAYMQLSSLTSDDSAVYFCARTRYGGNAMDYWGQGTSVTVSS

SEQ ID NO: 155 - protein, 117 aa, synthetic construct
  REGION 1..117: note = Synthetic
QVQLKESGPGQVAPSQSLSIACTVSGFSLSSYGVHWVRQPPGKGLEWLGVIWAGGSTNYDSALMSRLTISKDNSRTRVFLKMNSLQTDDTAIYYCARSLYGNSLDSWGPGTTLTVSS

SEQ ID NO: 156 - protein, 117 aa, synthetic construct
  REGION 1..117: note = Synthetic
QVQLKESGPGLVAPSQSLSITCTVSGLSLTSFGVHWIRQPPGKGLEWLGVIWAGGSTNYNSALMSRLSISKDNSKSQVYLKMHSLQTDDTAMYYCARSLYGNSFDYWGQGTALTVSS

SEQ ID NO: 157 - protein, 117 aa, synthetic construct
  REGION 1..117: note = Synthetic
QVQLKESGPGLVAPSQSLSITCTVSGFSLSSYGVNWVRQPPGKGLEWLAAIRSDGIITYNSVLKSRLRISKDNSKSQVFLKMNSLQTDDTAMFYCARWFRGNVLDYWGQGTSVTVSS

SEQ ID NO: 158 - protein, 118 aa, synthetic construct
  REGION 1..118: note = Synthetic
QVQLKESGPGLVAPSQSLSITCTVSGFSLISYGVHWVRQPPGKGLEWLGVIWAGGRTNYNSALMSRLSISKDNSKSQVFLKMNSLQTYDTAMYYCARDRYGGNSLDYWGQGTSVTVSS

SEQ ID NO: 159 - protein, 119 aa, synthetic construct
  REGION 1..119: note = Synthetic
DVQLVESGGGLVQPGGSRKLSCAASGFTFSTFGMHWVRQAPEKGLEWVAYITSGNSPIYFTDTVKGRFTISRDNPKNTLFLQMTSLGSEDTAVYYCARSSYYGNSMDYWGQGTSVTVSS

SEQ ID NO: 160 - protein, 118 aa, synthetic construct
  REGION 1..118: note = Synthetic
QVQLKESGPGLVAPSQSLSITCTISGFSLNTYGVHWVRQPPGKGLEWLVVMLSDGNTVYNSSLKSRLSLTKDNSKSQLLLKMNSLQTDDTAIYYCARHKAYGNAMDYWGQGTSVTVSS

SEQ ID NO: 161 - protein, 117 aa, synthetic construct
  REGION 1..117: note = Synthetic
QVQLKESGPGLVAPSQSLSITCTVSGFSLINYGVSWVRQPPGKGLEWLGVIWGDGNTNYQSALRSRLSIRKDTSKSQVFLKLNSVHTDGTATYYCAKVGRGNAMDHWGQGISVIVSS

SEQ ID NO: 162 - protein, 117 aa, synthetic construct
  REGION 1..117: note = Synthetic
QVQLKESGPGLVAPSQSLSITCTVSGFSLINYGVSWVRQPPGKGLEWLGVIRGDGNTNYQSALRSRLSIRKDTSKSQVFLKLNSVHTDGTATYYCAKVGRGNAMDHWGQGISVIVSS

SEQ ID NO: 163 - protein, 118 aa, synthetic construct
  REGION 1..118: note = Synthetic
EVQLQQSGPELVKPGASVKMSCKASGYTFTGFLMHWVKQKPGQGLEWIGYINPYNDGTKYSEKFKGKATLTSDKSSSTAFMELSSLTSDDSAVYYCARLDYGNAMDYWGQGTSVTVSS

SEQ ID NO: 164 - protein, 118 aa, synthetic construct
  REGION 1..118: note = Synthetic
QVQLKQSGPGLVQPSQSLSITCTVSDFSLTKYGVHWFRQSPGKGLEWLGVIWTGGNTDYNPALIPRLSFRKDNSKSQVFFKMNSLQSSDTAVYYCARNGYYGNAMDYWGQGTSVTVSS

SEQ ID NO: 165 - protein, 118 aa, synthetic construct
  REGION 1..118: note = Synthetic
EVQLQQSGPELVKPGASVKMSCKSSGYTFTGFLMHWVKQKPGQGLEWIGYINPYNDGTKYSEKFKGKATLTSDKSSSTAYMELSSLTSDDSAVYYCGRLDYGNAMDYWGQGTSVTVSS

SEQ ID NO: 166 - protein, 115 aa, synthetic construct
  REGION 1..115: note = Synthetic
DVQLQESGPGLVKPSQSLSLSCSVTGYSITSGYFWTWFRQFPGNKLEWMGYISYDGSNNYNPSLKNRISITRDTSKNQFFLKLNSVTTEDTATYYCASFRFFAYWGQGTLVTVSA

SEQ ID NO: 167 - protein, 119 aa, synthetic construct
  REGION 1..119: note = Synthetic
QVQLKESGPGLVAPSQSLSITCTVSGFSLSNYGVSWVRQPPGKGLEWLGVIWAGGNTNYNSALMSRLRISKDNSKSQVFLKMNSLQTDDTARYYCARHGYGKGNAMDNWGQGTSVTVSS

SEQ ID NO: 168 - protein, 118 aa, synthetic construct
  REGION 1..118: note = Synthetic
QVQLQQPGAEPVKPGASVKLSCKASGYSFTNFLTHWVRQRPGQGLEWIGEINPTNGRTYYNEKFKRKATLTVDKSSTTVYMQLSNLTPEDSAVFYCARIYYGNSMDYWGQGTLVTVSA

SEQ ID NO: 169 - protein, 120 aa, synthetic construct
  REGION 1..120: note = Synthetic
QVQLQQSGAELARPGASVKMSCKASGYTFPTYSIHWLKQGPGQGLEWIGYINPNTIYTNYNQKFKYKTTLTADKSSSTAYIQLSSLTSDDSAVYYCAREGYGRGNAMDYWGQGTSVTVSS

SEQ ID NO: 170 - protein, 117 aa, synthetic construct
  REGION 1..117: note = Synthetic
QVQLKESGPGLVAPSQSLSITCTVTGFSLSSYGVHWVRQPPGKGLEWLGVIWAGGSTNYDSALMSRLSISKDRSKSQVFLKMTSLQTDDTAMYYCARSLYGNSFDHWGQGTTLTVSS

SEQ ID NO: 171 - protein, 118 aa, synthetic construct
  REGION 1..118: note = Synthetic
EVQLQQSGPELVKPGASVKMSCKASGYTFTGYIIQWMKQKPGLGLEWIGFINPYNDGTKYNEQFKGKATLTSDKSSNAAYMELSSLTSEDSAVYYCARAYFGNSFAYWGQGTLVTVSA

SEQ ID NO: 172 - protein, 117 aa, synthetic construct
  REGION 1..117: note = Synthetic
QVQLKESGPGLVAPSQSLSITCTVSGFSLTSYGVHWVRQPPGKGLEWLGVIWAGGSTNYDSTLMSRLSISKDRSKSQVFLKMTSLQTDDTAMYYCARSLYGNSFDHWGQGTTLTVSS

SEQ ID NO: 173 - protein, 117 aa, synthetic construct
  REGION 1..117: note = Synthetic
QVQLKESGPGLVAPSQSLSITCTVSGFSLTSYGAHWVRQPPGKGLEWLGVIWAGGSTNYDSALMSRLSISKDRSKSQVFLKMTSLQTDDTAMYYCARSLYGNSFDHWGQGTTLTVSS

SEQ ID NO: 174 - protein, 118 aa, synthetic construct
  REGION 1..118: note = Synthetic
EVQLQQSGPELVKPGASVKMSCKASGYTFTGFLMHWVKQKPGQGLEWIGYINPYNDGTKYSERFKGKATLTSDKSSSTAYMELSSLTSDDSAVYYCARLDYGNAMDYWGQGTSVTVSS

SEQ ID NO: 175 - protein, 119 aa, synthetic construct
  REGION 1..119: note = Synthetic
EVQLVESGGGLVQPGGSLRLSCAASGFTFSTFGMHWVRQAPGKGLEWVSYITSGNSPIYFTDTVKGRFTISRDNAKNSLYLQMNSLRAEDTAVYYCARSSYYGNSMDYWGQGTLVTVSS

SEQ ID NO: 176 - protein, 119 aa, synthetic construct
  REGION 1..119: note = Synthetic
EVQLVESGGGLVQPGGSLRLSCAASGFTFSTFGMHWVRQAPGKGLEWVAYITSGNSPIYFTDTVKGRFTISRDNAKNSLYLQMNSLRAEDTAVYYCARSSYYGNSMDYWGQGTLVTVSS

SEQ ID NO: 177 - protein, 119 aa, synthetic construct
  REGION 1..119: note = Synthetic
EVQLVESGGGLVQPGGSLRLSCAASGFTFSTFGMHWVRQAPGKGLEWVAYITSGNSPIYFTDTVKGRFTISRDNAKNTLYLQMNSLRAEDTAVYYCARSSYYGNSMDYWGQGTLVTVSS

SEQ ID NO: 178 - protein, 113 aa, synthetic construct
  REGION 1..113: note = Synthetic
DIVMTQSPDSLAVSLGERATINCRSSQSLLNSGNRKNYLTWYQQKPGQPPKLLIYWASTRESGVPDRFSGSGSGTDFTLTISSLQAEDVAVYYCQNAYSYPFTFGGGTKLEIK

SEQ ID NO: 179 - protein, 113 aa, synthetic construct
  REGION 1..113: note = Synthetic
DTVMTQSPDSLAVSLGERATINCRSSQSLLNSGNRKNYLTWYQQKPGQPPKLLIYWASTRESGVPDRFSGSGSGTDFTLTISSVQAEDVAVYYCQNAYSYPFTFGGGTKLEIK

SEQ ID NO: 180 - protein, 113 aa, synthetic construct
  REGION 1..113: note = Synthetic
DTVMTQSPDSLAVSLGERVTLNCRSSQSLLNSGNRKNYLTWYQQKPGQPPKLLIYWASTRESGVPDRFSGSGSGTDFTLTISSVQAEDVAVYYCQNAYSYPFTFGGGTKLEIK

SEQ ID NO: 181 - protein, 120 aa, synthetic construct
  REGION 1..120: note = Synthetic
QVQLVQSGAEVKKPGASVKVSCKASGYTFTTYPIEWVRQAPGQRLEWMGNFHPYNDDTKYNEKFKGRVTITRDTSASTAYMELSSLRSEDTAVYYCARRAYGYPYAMDYWGQGTLVTVSS

SEQ ID NO: 182 - protein, 120 aa, synthetic construct
  REGION 1..120: note = Synthetic
QVQLVQSGAEVVKPGASVKVSCKASGYTFTTYPIEWMRQAPGQRLEWMGNFHPYNDDTKYNEKFKGRVTITVDTSASTAYMELSSLRSEDTAVYYCARRAYGYPYAMDYWGQGTLVTVSS

SEQ ID NO: 183 - protein, 120 aa, synthetic construct
  REGION 1..120: note = Synthetic
QVQLVQSGAEVVKPGASVKVSCKASGYTFTTYPIEWMKQAPGQRLEWMGNFHPYNDDTKYNEKFKGRVTITVDTSASTAYMEVSSLRSEDTAVYYCARRAYGYPYAMDYWGQGTLVTVSS

SEQ ID NO: 184 - protein, 120 aa, synthetic construct
  REGION 1..120: note = Synthetic
QVQLVQSGAEVVKPGASVKMSCKASGYTFTTYPIEWMKQAPGQRLEWMGNFHPYNDDTKYNEKFKGRVTLTVDTSASTVYLEVSSLRSEDTAVYYCARRAYGYPYAMDYWGQGTLVTVSS

SEQ ID NO: 185 - protein, 113 aa, synthetic construct
  REGION 1..113: note = Synthetic
DIVMTQSPDSLAVSLGERATINCKSSQSLLNSGNQKNYLTWYQQKPGQPPKLLIYRASSRESGVPDRFSGSGSGTDFTLTISSLQAEDVAVYYCQNDYIYPYTFGGGTKLEIK

SEQ ID NO: 186 - protein, 113 aa, synthetic construct
  REGION 1..113: note = Synthetic
DIVMTQSPDSLAVSLGERATISCKSSQSLLNSGNQKNYLTWYQQKPGQPPKLLIYRASSRESGVPDRFSGSGSGTDFTLTISSLQAEDVAVYYCQNDYIYPYTFGGGTKLEIK

SEQ ID NO: 187 - protein, 113 aa, synthetic construct
  REGION 1..113: note = Synthetic
DIVMTQSPDSLAVSLGERVTMSCKSSQSLLNSGNQKNYLTWYQQKPGQPPKLLIYRASSRESGVPDRFSGSGSGTDFTLTISSVQAEDVAVYYCQNDYIYPYTFGGGTKLEIK

SEQ ID NO: 188 - protein, 118 aa, synthetic construct
  REGION 1..118: note = Synthetic
QVQLVQSGAEVKKPGASVKVSCKASGYTFTGYIIQWVRQAPGQRLEWMGFINPYNDGTKYNEQFKGRVTITRDTSASTAYMELSSLRSEDTAVYYCARAYFGNSFAYWGQGTLVTVSS

SEQ ID NO: 189 - protein, 118 aa, synthetic construct
  REGION 1..118: note = Synthetic
QVQLVQSGAEVVKPGASVKVSCKASGYTFTGYIIQWMRQAPGQRLEWMGFINPYNDGTKYNEQFKGRVTITSDTSASAAYMELSSLRSEDTAVYYCARAYFGNSFAYWGQGTLVTVSS

SEQ ID NO: 190 - protein, 118 aa, synthetic construct
  REGION 1..118: note = Synthetic
QVQLVQSGAEVVKPGASVKVSCKASGYTFTGYIIQWMKQAPGQRLEWIGFINPYNDGTKYNEQFKGRATITSDTSASAAYMELSSLRSEDTAVYYCARAYFGNSFAYWGQGTLVTVSS

SEQ ID NO: 191 - protein, 118 aa, synthetic construct
  REGION 1..118: note = Synthetic
EVQLVQSGAEVVKPGASVKMSCKASGYTFTGYIIQWMKQAPGQRLEWIGFINPYNDGTKYNEQFKGRATLTSDTSASAAYMELSSLRSEDTAVYYCARAYFGNSFAYWGQGTLVTVSS

SEQ ID NO: 192 - protein, 113 aa, synthetic construct
  REGION 1..113: note = Synthetic
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNGYYFPFTFGGGTKLEIK

SEQ ID NO: 193 - protein, 113 aa, synthetic construct
  REGION 1..113: note = Synthetic
DIVMTQSPDSLAVSLGERATISCKSSQSLLNSGNQKNYLTWYQQKPGQPPKLLMYWASTRESGVPDRFSGSGSGTDFTLTISSLQAEDVAVYYCQNGYYFPFTFGGGTKLEIK

SEQ ID NO: 194 - protein, 113 aa, synthetic construct
  REGION 1..113: note = Synthetic
DIVMTQSPDSLAVSLGERATISCKSSQSLLNSGNQKNYLTWYQQKPGQPPKLLMYWASTRESGVPDRFSGSGSGTDFTLTISSGQAEDVAVYFCQNGYYFPFTFGGGTKLEIK

SEQ ID NO: 195 - protein, 113 aa, synthetic construct
  REGION 1..113: note = Synthetic
DIVMTQSPDSLAVSLGERVTMSCKSSQSLLNSGNQKNYLTWYQQKPGQPPKLLMYWASTRESGVPDRFSGSGSGTDFTLTISSGQAEDVAVYFCQNGYYFPFTFGGGTKLEIK

SEQ ID NO: 196 - protein, 119 aa, synthetic construct
  REGION 1..119: note = Synthetic
EVQLVESGGGLVQPGGSLRLSCAASGFTFSTFGMHWVRQAPGKGLEWVSYITSGQSPIYFTDTVKGRFTISRDNAKNSLYLQMNSLRAEDTAVYYCARSSYYGNSMDYWGQGTLVTVSS

SEQ ID NO: 197 - protein, 119 aa, synthetic construct
  REGION 1..119: note = Synthetic
EVQLVESGGGLVQPGGSLRLSCAASGFTFSTFGMHWVRQAPGKGLEWVSYITSGESPIYFTDTVKGRFTISRDNAKNSLYLQMNSLRAEDTAVYYCARSSYYGNSMDYWGQGTLVTVSS

SEQ ID NO: 198 - protein, 119 aa, synthetic construct
  REGION 1..119: note = Synthetic
EVQLVESGGGLVQPGGSLRLSCAASGFTFSTFGMHWVRQAPGKGLEWVSYITSGESPIYFTDTVKGRFTISRDNAKNSLYLQMNSLRAEDTAVYYCARSSYYGQSMDYWGQGTLVTVSS

SEQ ID NO: 199 - protein, 119 aa, synthetic construct
  REGION 1..119: note = Synthetic
EVQLVESGGGLVQPGGSLRLSCAASGFTFSTFGMHWVRQAPGKGLEWVSYITSGESPIYFTDTVKGRFTISRDNAKNSLYLQMNSLRAEDTAVYYCARSSYYGESMDYWGQGTLVTVSS

SEQ ID NO: 200 - protein, 119 aa, synthetic construct
  REGION 1..119: note = Synthetic
EVQLVESGGGLVQPGGSLRLSCAASGFTFSTFGMHWVRQAPGKGLEWVSYITSGESPIYFTDTVKGRFTISRDNAKNSLYLQMNSLRAEDTAVYYCARSSYYGNAMDYWGQGTLVTVSS

SEQ ID NO: 201 - protein, 113 aa, synthetic construct
  REGION 1..113: note = Synthetic
DIVMTQSPDSLAVSLGERATINCRSSQSLLESGNRKNYLTWYQQKPGQPPKLLIYWASTRESGVPDRFSGSGSGTDFTLTISSLQAEDVAVYYCQNAYSYPFTFGGGTKLEIK

SEQ ID NO: 202 - protein, 113 aa, synthetic construct
  REGION 1..113: note = Synthetic
DIVMTQSPDSLAVSLGERATINCRSSQSLLNAGNRKNYLTWYQQKPGQPPKLLIYWASTRESGVPDRFSGSGSGTDFTLTISSLQAEDVAVYYCQNAYSYPFTFGGGTKLEIK

SEQ ID NO: 203 - protein, 113 aa, synthetic construct
  REGION 1..113: note = Synthetic
DIVMTQSPDSLAVSLGERATINCKSSQSLLNAGNQKNYLTWYQQKPGQPPKLLIYRASSRESGVPDRFSGSGSGTDFTLTISSLQAEDVAVYYCQNDYIYPYTFGGGTKLEIK

SEQ ID NO: 204 - protein, 113 aa, synthetic construct
  REGION 1..113: note = Synthetic
DIVMTQSPDSLAVSLGERATINCKSSQSLLESGNQKNYLTWYQQKPGQPPKLLIYRASSRESGVPDRFSGSGSGTDFTLTISSLQAEDVAVYYCQNDYIYPYTFGGGTKLEIK

SEQ ID NO: 205 - protein, 118 aa, synthetic construct
  REGION 1..118: note = Synthetic
QVQLVQSGAEVKKPGASVKVSCKASGYTFTGYIIQWVRQAPGQRLEWMGFINPYNDDTKYNEQFKGRVTITRDTSASTAYMELSSLRSEDTAVYYCARAYFGNAFAYWGQGTLVTVSS

SEQ ID NO: 206 - protein, 113 aa, synthetic construct
  REGION 1..113: note = Synthetic
DIVMTQSPDSLAVSLGERATINCKSSQSLLNAGNQKNYLTWYQQKPGQPPKLLIYWASTRESGVPDRFSGSGSGTDFTLTISSLQAEDVAVYYCQNAYYFPFTFGGGTKLEIK

SEQ ID NO: 207 - protein, 113 aa, synthetic construct
  REGION 1..113: note = Synthetic
DIVMTQSPDSLAVSLGERATINCKSSQSLLESGNQKNYLTWYQQKPGQPPKLLIYWASTRESGVPDRFSGSGSGTDFTLTISSLQAEDVAVYYCQEGYYFPFTFGGGTKLEIK

SEQ ID NO: 208 - protein, 17 aa, synthetic construct
  REGION 1..17: note = Synthetic
KSSQSLLNSGNQRNYLT

SEQ ID NO: 209 - protein, 17 aa, synthetic construct
  REGION 1..17: note = Synthetic
KSSQSLLNSGNLKNYLT

SEQ ID NO: 210 - protein, 17 aa, synthetic construct
  REGION 1..17: note = Synthetic
KSSQSLLNSGNQKNYLT

SEQ ID NO: 211 - protein, 17 aa, synthetic construct
  REGION 1..17: note = Synthetic
KSSQSLLNGGNQKNYLA

SEQ ID NO: 212 - protein, 17 aa, synthetic construct
  REGION 1..17: note = Synthetic
KSNQSLLNSGNQKKYLT

SEQ ID NO: 213 - protein, 17 aa, synthetic construct
  REGION 1..17: note = Synthetic
KSSQSLFNSGNQRNYLT

SEQ ID NO: 214 - protein, 17 aa, synthetic construct
  REGION 1..17: note = Synthetic
RSSMSLFNSGNQKSYLS

SEQ ID NO: 215 - protein, 17 aa, synthetic construct
  REGION 1..17: note = Synthetic
RSIQSLLNSGNQKNYLS

SEQ ID NO: 216 - protein, 17 aa, synthetic construct
  REGION 1..17: note = Synthetic
RSSQSLLNSGNRKNYLT

SEQ ID NO: 217 - protein, 17 aa, synthetic construct
  REGION 1..17: note = Synthetic
RSTQSLFNSGNQKNYLT

SEQ ID NO: 218 - protein, 17 aa, synthetic construct
  REGION 1..17: note = Synthetic
KPSQSLLNSGNQKNYLA

SEQ ID NO: 219 - protein, 17 aa, synthetic construct
  REGION 1..17: note = Synthetic
RSTQSLFNSGNQRNYLT

SEQ ID NO: 220 - protein, 17 aa, synthetic construct
  REGION 1..17: note = Synthetic
KSSQSLLNSGNQKNYLA

SEQ ID NO: 221 - protein, 17 aa, synthetic construct
  REGION 1..17: note = Synthetic
KSSQSVFNSGNQKNYLT

SEQ ID NO: 222 - protein, 17 aa, synthetic construct
  REGION 1..17: note = Synthetic
RSSQSLLNGGNQKNYLT

SEQ ID NO: 223 - protein, 17 aa, synthetic construct
  REGION 1..17: note = Synthetic
KSSQSLLNSGNQRNYLS

SEQ ID NO: 224 - protein, 17 aa, synthetic construct
  REGION 1..17: note = Synthetic
KSSQSLFNSGNQKNYLT

SEQ ID NO: 225 - protein, 17 aa, synthetic construct
  REGION 1..17: note = Synthetic
RSSQSLFNSGNQRNYLT

SEQ ID NO: 226 - protein, 17 aa, synthetic construct
  REGION 1..17: note = Synthetic
KSTQSLLNSGNQRNYLT

SEQ ID NO: 227 - protein, 7 aa, synthetic construct
  REGION 1..7: note = Synthetic
WASTRES

SEQ ID NO: 228 - protein, 7 aa, synthetic construct
  REGION 1..7: note = Synthetic
GASTRES

SEQ ID NO: 229 - protein, 7 aa, synthetic construct
  REGION 1..7: note = Synthetic
RASSRES

SEQ ID NO: 230 - protein, 7 aa, synthetic construct
  REGION 1..7: note = Synthetic
WAFTRES

SEQ ID NO: 231 - protein, 7 aa, synthetic construct
  REGION 1..7: note = Synthetic
WSSTRDS

SEQ ID NO: 232 - protein, 7 aa, synthetic construct
  REGION 1..7: note = Synthetic
WASTRDS

SEQ ID NO: 233 - protein, 7 aa, synthetic construct
  REGION 1..7: note = Synthetic
WSSTRES

SEQ ID NO: 234 - protein, 5 aa, synthetic construct
  REGION 1..5: note = Synthetic
NYLLE

SEQ ID NO: 235 - protein, 5 aa, synthetic construct
  REGION 1..5: note = Synthetic
SYGVS

SEQ ID NO: 236 - protein, 5 aa, synthetic construct
  REGION 1..5: note = Synthetic
SFGMN

SEQ ID NO: 237 - protein, 5 aa, synthetic construct
  REGION 1..5: note = Synthetic
TNAMN

SEQ ID NO: 238 - protein, 5 aa, synthetic construct
  REGION 1..5: note = Synthetic
TYSIH

SEQ ID NO: 239 - protein, 5 aa, synthetic construct
  REGION 1..5: note = Synthetic
NYGMS

SEQ ID NO: 240 - protein, 5 aa, synthetic construct
  REGION 1..5: note = Synthetic
NYGMN

SEQ ID NO: 241 - protein, 5 aa, synthetic construct
  REGION 1..5: note = Synthetic
KYGMN

SEQ ID NO: 242 - protein, 5 aa, synthetic construct
  REGION 1..5: note = Synthetic
TYPIE

SEQ ID NO: 243 - protein, 5 aa, synthetic construct
  REGION 1..5: note = Synthetic
NYLIE

SEQ ID NO: 244 - protein, 5 aa, synthetic construct
  REGION 1..5: note = Synthetic
SYGVH

SEQ ID NO: 245 - protein, 5 aa, synthetic construct
  REGION 1..5: note = Synthetic
SFGVH

SEQ ID NO: 246 - protein, 5 aa, synthetic construct
  REGION 1..5: note = Synthetic
TFGMH

SEQ ID NO: 247 - protein, 5 aa, synthetic construct
  REGION 1..5: note = Synthetic
TYGVH

SEQ ID NO: 248 - protein, 5 aa, synthetic construct
  REGION 1..5: note = Synthetic
NYGVS

SEQ ID NO: 249 - protein, 5 aa, synthetic construct
  REGION 1..5: note = Synthetic
GFLMH

SEQ ID NO: 250 - protein, 5 aa, synthetic construct
  REGION 1..5: note = Synthetic
KYGVH

SEQ ID NO: 251 - protein, 5 aa, synthetic construct
  REGION 1..5: note = Synthetic
SGYFW

SEQ ID NO: 252 - protein, 5 aa, synthetic construct
  REGION 1..5: note = Synthetic
NFLTH

SEQ ID NO: 253 - protein, 5 aa, synthetic construct
  REGION 1..5: note = Synthetic
GYIIQ

SEQ ID NO: 254 - protein, 5 aa, synthetic construct
  REGION 1..5: note = Synthetic
SYGAH

SEQ ID NO: 255 - protein, 17 aa, synthetic construct
  REGION 1..17: note = Synthetic
EINPGNGGSNYNEKFKG

SEQ ID NO: 256 - protein, 16 aa, synthetic construct
  REGION 1..16: note = Synthetic
VIWGDGNTIYHSALKS

SEQ ID NO: 257 - protein, 17 aa, synthetic construct
  REGION 1..17: note = Synthetic
FISGGSNTIHYLDTVKG

SEQ ID NO: 258 - protein, 19 aa, synthetic construct
  REGION 1..19: note = Synthetic
RIRSKSNNYATYYADSVKD

SEQ ID NO: 259 - protein, 17 aa, synthetic construct
  REGION 1..17: note = Synthetic
YINPSTIYTNYNQKFKY

SEQ ID NO: 260 - protein, 17 aa, synthetic construct
  REGION 1..17: note = Synthetic
TFSYGDSHNYYSDSVKG

SEQ ID NO: 261 - protein, 17 aa, synthetic construct
  REGION 1..17: note = Synthetic
WINANTGEPTYAEEFKG

SEQ ID NO: 262 - protein, 17 aa, synthetic construct
  REGION 1..17: note = Synthetic
WISTNTGEPTYAEEFKG

SEQ ID NO: 263 - protein, 17 aa, synthetic construct
  REGION 1..17: note = Synthetic
NFHPYNDDTKYNEKFKG

SEQ ID NO: 264 - protein, 17 aa, synthetic construct
  REGION 1..17: note = Synthetic
VINPGRSGTNYNEKFKG

SEQ ID NO: 265 - protein, 16 aa, synthetic construct
  REGION 1..16: note = Synthetic
VIWAGGSTNYDSALMS

SEQ ID NO: 266 - protein, 16 aa, synthetic construct
  REGION 1..16: note = Synthetic
VIWAGGSTNYNSALMS

SEQ ID NO: 267 - protein, 16 aa, synthetic construct
  REGION 1..16: note = Synthetic
VIWAGGRTNYNSALMS

SEQ ID NO: 268 - protein, 17 aa, synthetic construct
  REGION 1..17: note = Synthetic
YITSGNSPIYFTDTVKG

SEQ ID NO: 269 - protein, 16 aa, synthetic construct
  REGION 1..16: note = Synthetic
VMLSDGNTVYNSSLKS

SEQ ID NO: 270 - protein, 16 aa, synthetic construct
  REGION 1..16: note = Synthetic
VIWGDGNTNYQSALRS

SEQ ID NO: 271 - protein, 16 aa, synthetic construct
  REGION 1..16: note = Synthetic
VIRGDGNTNYQSALRS

SEQ ID NO: 272 - protein, 17 aa, synthetic construct
  REGION 1..17: note = Synthetic
YINPYNDGTKYSEKFKG

SEQ ID NO: 273 - protein, 16 aa, synthetic construct
  REGION 1..16: note = Synthetic
VIWTGGNTDYNPALIP

SEQ ID NO: 274 - protein, 16 aa, synthetic construct
  REGION 1..16: note = Synthetic
YISYDGSNNYNPSLKN

SEQ ID NO: 275 - protein, 16 aa, synthetic construct
  REGION 1..16: note = Synthetic
VIWAGGNTNYNSALMS

SEQ ID NO: 276 - protein, 17 aa, synthetic construct
  REGION 1..17: note = Synthetic
EINPTNGRTYYNEKFKR

SEQ ID NO: 277 - protein, 17 aa, synthetic construct
  REGION 1..17: note = Synthetic
YINPNTIYTNYNQKFKY

SEQ ID NO: 278 - protein, 17 aa, synthetic construct
  REGION 1..17: note = Synthetic
FINPYNDGTKYNEQFKG

SEQ ID NO: 279 - protein, 16 aa, synthetic construct
  REGION 1..16: note = Synthetic
VIWAGGSTNYDSTLMS

SEQ ID NO: 280 - protein, 17 aa, synthetic construct
  REGION 1..17: note = Synthetic
YINPYNDGTKYSERFKG

SEQ ID NO: 281 - protein, 9 aa, synthetic construct
  REGION 1..9: note = Synthetic
IYYGNSFAY

SEQ ID NO: 282 - protein, 10 aa, synthetic construct
  REGION 1..10: note = Synthetic
QGLYGHAMDY

SEQ ID NO: 283 - protein, 9 aa, synthetic construct
  REGION 1..9: note = Synthetic
LALGNAMDY

SEQ ID NO: 284 - protein, 12 aa, synthetic construct
  REGION 1..12: note = Synthetic
GAYYGNSKAFDY

SEQ ID NO: 285 - protein, 11 aa, synthetic construct
  REGION 1..11: note = Synthetic
EGYGRGNAMDY

SEQ ID NO: 286 - protein, 9 aa, synthetic construct
  REGION 1..9: note = Synthetic
FGRGNTMDY

SEQ ID NO: 287 - protein, 9 aa, synthetic construct
  REGION 1..9: note = Synthetic
LTRGNSFDY

SEQ ID NO: 288 - protein, 9 aa, synthetic construct
  REGION 1..9: note = Synthetic
LVRGNSFDF

SEQ ID NO: 289 - protein, 11 aa, synthetic construct
  REGION 1..11: note = Synthetic
RAYGYPYAMDY

SEQ ID NO: 290 - protein, 10 aa, synthetic construct
  REGION 1..10: note = Synthetic
TRYGGNAMDY

SEQ ID NO: 291 - protein, 9 aa, synthetic construct
  REGION 1..9: note = Synthetic
SLYGNSLDS

SEQ ID NO: 292 - protein, 9 aa, synthetic construct
  REGION 1..9: note = Synthetic
SLYGNSFDY

SEQ ID NO: 293 - protein, 10 aa, synthetic construct
  REGION 1..10: note = Synthetic
DRYGGNSLDY

SEQ ID NO: 294 - protein, 10 aa, synthetic construct
  REGION 1..10: note = Synthetic
SSYYGNSMDY

SEQ ID NO: 295 - protein, 10 aa, synthetic construct
  REGION 1..10: note = Synthetic
HKAYGNAMDY

SEQ ID NO: 296 - protein, 9 aa, synthetic construct
  REGION 1..9: note = Synthetic
VGRGNAMDH

SEQ ID NO: 297 - protein, 9 aa, synthetic construct
  REGION 1..9: note = Synthetic
LDYGNAMDY

SEQ ID NO: 298 - protein, 10 aa, synthetic construct
  REGION 1..10: note = Synthetic
NGYYGNAMDY

SEQ ID NO: 299 - protein, 6 aa, synthetic construct
  REGION 1..6: note = Synthetic
FRFFAY

SEQ ID NO: 300 - protein, 11 aa, synthetic construct
  REGION 1..11: note = Synthetic
HGYGKGNAMDN

SEQ ID NO: 301 - protein, 9 aa, synthetic construct
  REGION 1..9: note = Synthetic
IYYGNSMDY

SEQ ID NO: 302 - protein, 9 aa, synthetic construct
  REGION 1..9: note = Synthetic
SLYGNSFDH

SEQ ID NO: 303 - protein, 9 aa, synthetic construct
  REGION 1..9: note = Synthetic
AYFGNSFAY

SEQ ID NO: 304 - protein, 17 aa, synthetic construct
  REGION 1..17: note = Synthetic
KSSQSLLNAGNQKNYLT

SEQ ID NO: 305 - protein, 17 aa, synthetic construct
  REGION 1..17: note = Synthetic
KSSQSLLESGNQKNYLT

SEQ ID NO: 306 - protein, 17 aa, synthetic construct
  REGION 1..17: note = Synthetic
FINPYNDDTKYNEQFKG

SEQ ID NO: 307 - protein, 9 aa, synthetic construct
  REGION 1..9: note = Synthetic
AYFGNAFAY

SEQ ID NO: 308 - protein, 17 aa, synthetic construct
  REGION 1..17: note = Synthetic
RSSQSLLESGNRKNYLT

SEQ ID NO: 309 - protein, 17 aa, synthetic construct
  REGION 1..17: note = Synthetic
RSSQSLLNAGNRKNYLT

SEQ ID NO: 310 - protein, 17 aa, synthetic construct
  REGION 1..17: note = Synthetic
YITSGQSPIYFTDTVKG

SEQ ID NO: 311 - protein, 17 aa, synthetic construct
  REGION 1..17: note = Synthetic
YITSGESPIYFTDTVKG

SEQ ID NO: 312 - protein, 10 aa, synthetic construct
  REGION 1..10: note = Synthetic
SSYYGQSMDY

SEQ ID NO: 313 - protein, 10 aa, synthetic construct
  REGION 1..10: note = Synthetic
SSYYGESMDY

SEQ ID NO: 314 - protein, 10 aa, synthetic construct
  REGION 1..10: note = Synthetic
SSYYGNAMDY

(19) United States
(12) Patent Application Publication
(10) Pub. No.: US 2025/0339550 A1
(43) Pub. Date: Nov. 6, 2025

(54) ANTIBODY-DRUG CONJUGATES TARGETING HUMAN CLAUDIN 18.2

(71) Applicant: LaNova Medicines Limited Company, Shanghai (CN)

(72) Inventor: RunSheng LI, Shanghai (CN)

(21) Appl. No.: 19/232,513

(22) Filed: Jun. 9, 2025

Related U.S. Application Data
(63) Continuation of application No. 18/065,371, filed on Dec. 13, 2022, now Pat. No. 12,324,936.
Application No. 18/065,371 is a continuation of application No. 17/530,209, filed on Nov. 18, 2021, now Pat. No. 11,554,177.
Application No. 17/530,209 is a continuation of application No. PCT/CN2020/126780, filed on Nov. 5, 2020.

(30) Foreign Application Priority Data
Nov. 5, 2019 (WO) PCT/CN2019/115760

(51) Int. Cl. A61P 35/00; A61K 47/68

(52) U.S. Cl. CPC A61K 47/68031; A61K 47/6817; A61K 47/6849; A61P 35/00

(57) ABSTRACT

Provided are antibody-drug conjugates containing a drug moiety attached to an antibody or fragment thereof having binding specificity to the wild-type human claudin 18.2 (CLDN18.2) protein. The antibody or the fragment thereof binds to the β3-β4 loop (residues 45-63 of SEQ ID NO: 30, NYQGLWRSCVRESSGFTEC) and the β5 strand (residues 169-172 of SEQ ID NO: 30, YTFG) of CLDN18.2.

### CROSS REFERENCE TO RELATED APPLICATIONS

This application is a continuation of U.S. application Ser. No. 18/065,371, filed Dec. 13, 2022, which is a continuation of U.S. application Ser. No. 17/530,209, filed Nov. 18, 2021, now U.S. Pat. No. 11,554,177, issued Jan. 17, 2023, which is a continuation of International Application Number PCT/CN2020/126780, filed Nov. 5, 2020, which claims priority to PCT/CN2019/115760, filed Nov. 5, 20219, the contents of each of which is incorporated herein by reference in its entirety in the present disclosure.

### SEQUENCE LISTING

The material in the accompanying sequence listing is hereby incorporated by reference in its entirety. The accompanying file, named 71TD_308092.xml was created on Jun. 9, 2025, and is 384 kilobytes in size.

### BACKGROUND

Claudins, such as claudin 18.2, are considered promising targets for cancer immunotherapy. Claudins are a family of proteins that form the important components of the tight cell junctions. They establish a paracellular barrier which controls the flow of molecules between the cells. The proteins have N-terminus and a C-terminus in the cytoplasm. Different claudins are expressed on different tissues, their altered function has linked to formation of cancers of respective tissues. Claudin-1 is expressed in colon cancer, claudin-18 is expressed in gastric cancer, and claudin-10 is expressed in hepatocellular carcinoma.

Claudin-18 has two isoforms, isoform 1 and isoform 2. Isoform 2 (Claudin 18.2 or CLDN18.2) is a highly selective cell lineage marker. Claudin 18.2's expression in normal tissues is strictly confined to differentiated epithelial cells of the gastric mucosa, but it was absent from the gastric stem cell zone. Claudin 18.2 was retained on malignant transformation and was expressed in a significant proportion of primary gastric cancers and its metastases. Frequently ectopic activation of claudin 18.2 was also found in pancreatic, esophageal, ovarian, and lung tumors. These data suggested that CLDN18.2 has highly restricted expression pattern in normal tissues, with frequent ectopic activation in a diversity of human cancers.

### SUMMARY

Anti-claudin 18.2 antibodies are discovered herein that selectively bind to wild-type claudin 18.2 and a common mutant M149L, and do not bind to other claudin 18 isoforms, such as claudin 18.1. In a surprising and unexpected discovery, the present disclosure demonstrates that these antibodies are highly effective in inducing receptor-mediated antibody internalization, in particular when compared to IMAB362 (claudiximab), a lead anti-claudin 18.2 antibody under clinical development. Therefore, when conjugated to a drug moiety, these antibodies are capable of efficiently delivering the drug into target cells, such as cancer cells overexpressing the claudin 18.2 protein.

The greatly increased ability to induce receptor-mediated antibody internalization of the presently disclosed antibodies may be attributed to the way these antibodies bind to the claudin 18.2 protein. As demonstrated in Example 14 and illustrated in FIG. 20, amino acid residues on the claudin 18.2 protein that are important for the binding to the antibodies include those that are important for stabilizing the conformation of the extracellular loops (e.g., W30, L49, W50, C53, C63 and R80). More important, the residues that are involved in binding to the antibodies are contemplated to include N45, Y46, G48, V54, R55, E56, S58, F60, and E62, which are located between the B3 and B4 strands of the first extracellular loop, and Y169 and G172, which are in β5 of the second extracellular loop. By contrast, it is believed that known anti-claudin 18.2 antibodies only bind to one of the extracellular loops.

In accordance with one embodiment of the present disclosure, provided is an antibody-drug conjugate, comprising a drug moiety covalently attached to an antibody or fragment thereof having binding specificity to a wild-type human claudin 18.2 (CLDN18.2) protein, wherein the antibody or the fragment thereof binds to the B3-β4 loop and the β5 strand of CLDN18.2. The B3-4 loop consists of residues 45-63 of SEQ ID NO:30 (NYQGLWRSCVRESSGFTEC), and the B5 strand consists of residues 169-172 of SEQ ID NO: 30 (YTFG).

In some embodiments, the ratio of the number of drug moieties to the number of antibody or fragment is 1:1 to 20:1. In some embodiments, the ratio is 2:1 to 10:1. In some embodiments, the ratio is 2:1 to 6:1. In some embodiments, the ratio is about 2:1, 2.5:1, 3:1, 3.5:1, 4:1, 4.5:1 or 5:1.

In some embodiments, the antibody or the fragment thereof does not bind to β1 and β2, or binds to β1 or B2 at an affinity that is at least 10 fold lower than to the B3-β4 loop or the B5 strand. In some embodiments, the antibody or the fragment thereof does not bind to the CLDN18.1 protein or binds to CLDN18.1 at an affinity that is at least 10 fold lower than to CLDN18.2.

In some embodiments, the antibody or the fragment thereof binds to the CLDN18.2 M149L mutant at an affinity that is at least 1% of the affinity to the wild-type CLDN18.2 protein.

In some embodiments, the antibody or the fragment thereof binds to at least an amino acid residue selected from the group consisting of N45, Y46, G48, V54, R55, E56, S58, F60, and E62; and at least an amino acid residue selected from the group consisting of Y169 and G172, of SEQ ID NO:30.

The drug moiety may be a cytotoxic or cytostatic agent, an immunosuppressive agent, a radioisotope, a toxin, or the like. The drug moiety, once released in cancer cell, can inhibit the multiplication of the cancer cell, or causing apoptosis in the cancer cell. Examples of drug moieties are selected from the group consisting of DM1 (maytansine, N2′-deacetyl-N2′-(3-mercapto-1-oxopropyl)- or N2′-deacetyl-N2′-(3-mercapto-1-oxopropyl)-maytansine), mc-MMAD (6-maleimidocaproyl-monomethylauristatin-D or N-methyl-L-valyl-N-[(1S,2R)-2-methoxy-4-[(2S)-2-[(1R,2R)-1-methoxy-2-methyl-3-oxo-3-[[(1S)-2-phenyl-1-(2-thiazolyl)ethyl]amino]propyl]-1-pyrrolidinyl]-1-[(1S)-1-methylpropyl]-4-oxobutyl]-N-methyl-(9Cl)-L-valinamide), mc-MMAF (maleimidocaproyl-monomethylauristatin For N-[6-(2,5-dihydro-2,5-dioxo-1H-pyrrol-1-yl)-1-oxohexyl]-N-methyl-L-valyl-L-valyl-(3R,4S,5S)-3-methoxy-5-methyl-4-(methylamino) heptanoyl-(αR, βR,2S)-β-methoxy-α-methyl-2-pyrrolidinepropanoyl-L-phenylalanine) and mc-Val-Cit-PABA-MMAE (6-maleimidocaproyl-ValcCit-(p-aminobenzyloxycarbonyl)-monomethylauristatin E or N-[[[4-[[N-[6-(2,5-dihydro-2,5-dioxo-1H-pyrrol-1-yl)-1-oxohexyl]-L-valyl-N5-(aminocarbonyl)-L-ornithyl]amino]phenyl]methoxy]carbonyl]-N-methyl-L-valyl-N-[(1S,2R)-4-[(2S)-2-[(1R,2R)-3-[[(1R,2S)-2-hydroxy-1-methyl-2-phenylethyl]amino]-1-methoxy-2-methyl-3-oxopropyl]-1-pyrrolidinyl]-2-methoxy-1-[(1S)-1-methylpropyl]-4-oxobutyl]-N-methyl-L-valinamide). DM1 is a derivative of the tubulin inhibitor maytansine while MMAD, MMAE, and MMAF are auristatin derivatives.

Methods and uses for the treatment of diseases and conditions are also provided. In one embodiment, provided is a method of treating cancer in a patient in need thereof, comprising administering to the patient the antibody-drug conjugate of the present disclosure.

### BRIEF DESCRIPTION OF THE DRAWINGS

FIG. 1 shows that the mouse sera from all mice after DNA immunisation have high titration reacted with HEK293 cells transfected with CLD 18A2 by flow cytometry, CLD 18A1 as negtive control.

FIG. 2 shows that the hybridoma supernatants can bind to HEK293 cells transfected with human CLD18A2 by cell ELISA or flow cytometry.

FIG. 3 shows that the purified murine antibodies can bind to MKN45 cells transfected with human CLD18A2 by flow cytometry with high EC50, compared with positive reference antibody.

FIG. 4 shows that the purified murine antibodies can bind to SU620 cells endogenously expressing human CLD18A2 bearing M149L mutation by flow cytometry with high EC50, while the reference antibody did not.

FIG. 5 shows that the purified murine antibodies can bind to HEK293 cells transfected with mouse CLD18A2 by flow cytometry with high EC50.

FIG. 6 shows that the purified murine antibodies can bind to HEK293 cells transfected with cyno CLD18A2 by flow cytometry with high EC50.

FIG. 7 shows that the purified murine antibodies can bind to HEK293 cells transfected with human CLD18A2 by flow cytometry with high EC50.

FIG. 8 shows that the chimeric antibodies can bind to MKN45 cells transfected with human CLD18A2 by flow cytometry with high EC50, compared with positive reference antibody.

FIG. 9 shows that the chimeric antibodies can not bind to MKN45 cells transfected with human CLD18A1 by flow cytometry.

FIG. 10 shows that humanized antibodies can bind to MKN45 cells transfected with human CLD18A2 by flow cytometry with high EC50, compared with positive reference antibody.

FIG. 11 shows that the humanized antibodies cannot bind to MKN45 cells transfected with human CLD18A1 by flow cytometry.

FIG. 12 shows that humanized antibodies with CDR mutation can bind to MKN45 cells transfected with human CLD18A2 by flow cytometry with high EC50, compared with positive reference antibody.

FIG. 13 shows that the humanized antibodies with CDR mutation cannot bind to MKN45 cells transfected with human CLD18A1 by flow cytometry.

FIG. 14 shows that the de-risked variants had potent binding to cell surface CLD18A2.

FIG. 15 shows that certain mutations of CLD18A2 have significant effect on the indicated antibodies binding to HEK293 cells transfected these mutants, suggesting that these amino acid residues constitute at least part of the epitope.

FIG. 16 shows that antibodies 4F11E2, 72C1B6A3 and 120B7B2 had superior binding in both claudin 18.2 high and low CHO-K1 cells, as compared to 175D10.

FIG. 17 shows the potent ADCC testing results of 4F11E2, 72C1B6A3 and 120B7B2 using 175D10 antibody as a reference.

FIG. 18 shows that the S239D/1332E versions of the 4F11E2, 72C1B6A3 and 120B7B2 outperformed the 175D10 counterpart in the ADCC assays.

FIG. 19 shows that 4F11E2, 72C1B6A3 and 120B7B2 also had better ADCP effects than 175D10.

FIG. 20 illustrates the 3D and motif structures of claudin proteins.

FIG. 21 shows the internalizaiton results of tested chimeric antibodies as compared to reference antibody IMAB362 on CHO cells expressing claudin 18.2.

FIG. 22 shows the internalization results of tested humanized antibodies as compared to reference antibody IMAB362 on CHO cells expressing claudin 18.2.

FIG. 23 shows the internalizaiton results of tested humanized antibodies as compared to reference antibody IMAB362 on MKN45 cells expressing claudin 18.2.

FIG. 24 shows the binding affinities of the antibodies and their drug conjugates.

FIG. 25A-C show the cell toxicity of the tested antibody-MMAE conjugates upon internalzation in DAN-G, NUGC or SCG-7901 transfectants.

FIG. 26 shows the cell toxicity of the tested antibody-MMAE conjugates upon internalzation in SNU620 endogenously expressing human claudin 18.2.

FIG. 27 compares antibody-drug conjugate with antibody alone in reducing tumor growth in test animals.

FIG. 28 shows the average or individual tumor reduction effects of the antibody-drug conjugate.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg2+normally found in a cell.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG₁, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence For W-G-X-G, where X is any amino acid.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CK regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

As used herein, “percent humanization” is calculated by determining the number of framework amino acid differences (i.e., non-CDR difference) between the humanized domain and the germline domain, subtracting that number from the total number of amino acids, and then dividing that by the total number of amino acids and multiplying by 100.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Anti-Claudin 18.2 Antibodies and Fragments

The present disclosure provides anti-claudin 18.2 antibodies with high affinity to both the wild-type claudin 18.2 and a common mutant, M149L (the SU620 cell endogenously expressing this mutation). To the best knowledge of the inventors, all currently known anti-claudin 18.2 proteins do not bind to this mutant. The antibodies of the present disclosure, therefore, have the unique advantage of being capable of targeting both the wild-type and the M149L mutant claudin 18.2 protein. This advantage is important because a significant portion of cancer patients harbor this common mutation. It is also worth noting that the antibodies of the present disclosure do not bind to the other claudin 18 isoform, claudin 18.1 (or binds claudin 18.1 at a much lower affinity).

The antibodies and fragments of the present disclosure exhibited superior properties even when using a clinical candidate as a reference. 175D10 (IMAB362; claudiximab) is currently undergoing phase III clinical trials for treating gastric and gastroesophageal junction adenocarcinoma. The instant antibodies and fragment not only showed stronger binding activities, they also exhibited higher ADCC and ADCP activities under various different conditions, as compared to 175D10.

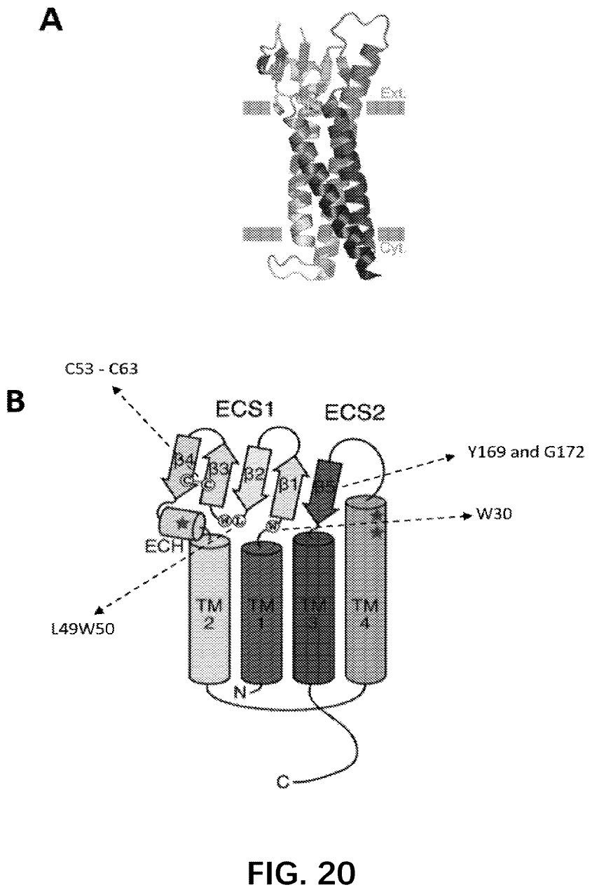

Also important, the present disclosure demonstrates that these antibodies are highly effective in inducing receptor-mediated antibody internalization, even when compared to IMAB362. The greatly increased ability to induce receptor-mediated antibody internalization of the presently disclosed antibodies, it is contemplated, can be attributed to how these antibodies bind to the claudin 18.2 protein. As demonstrated in Example 14 and illustrated in FIG. 20, amino acid residues on the claudin 18.2 protein that are important for the binding to the antibodies include those that are important for stabilizing the conformation of the extracellular loops (e.g., W30, L49, W50, C53, C63 and R80). W30, L49 and W50 are part of the W-LW-C-C consensus motif that helps to stabilize the conformation of loop 1. C53 and C63 form an inter-beta-strand disulfide bond. R80 is likely important for maintaining the interaction between parallel claudin 18.2 molecules on the cell surface, or for stabilizing the conformation of loop 1.

Also important for the antibody binding are residues N45, Y46, G48, V54, R55, E56, S58, F60, E62, Y169 and G172. Among them, N45, Y46, G48, V54, R55, E56, S58, F60 and E62 are located within the β3 strand, or through C63 in the β4 strand. This region, consisting of residues 45-63 of SEQ ID NO:30 (NYQGLWRSCVRESSGFTEC), is hereby referred to as the “β3 to β4 loop,” which is part of the first extracellular loop (loop 1) of claudin 18.2. Y169 and G172, by contrast, are part of the β5 strand (residues 169-172 of SEQ ID NO:30; YTFG) of the second extracellular loop (loop 2).

The greatly increased activity by the presently disclosed antibodies to induce receptor-mediated antibody internalization, it is contemplated, is due to their ability to bind to residues in both the β3 to β4 loop and the β5 strand. In this context, it is believed that known anti-claudin 18.2 antibodies only bind to one of the loops.

The experimental data also show that the presently disclosed antibodies have higher binding specificity and improved ADCC and ADCP as compared to known ones.

### Human Claudin 18.2 Sequence

| Name | Sequence (SEQ ID NO: 30) | |
|---|---|---|
| Human | 1 | MAVTACQGLG FVVSLIGIAG IIAATCMDQW STQDLYNNPV TAVFNYQGLW |
| Claudin 18.2 | | ------ |
| (NP_001002026) | 51 | RSCVRESSGF TECRGYFTLL GLPAMLQAVR ALMIVGIVLG AIGLLVSIFA |
| | | -------------- |
| | | β3-β4 loop |
| | 101 | LKCIRIGSME DSAKANMTLT SGIMFIVSGL CAIAGVSVFA NMLVTNFWMS |
| | 151 | TANMYTGMGG MVQTVQTRYT FGAALFVGWV AGGLTLIGGV MMCIACRGLA |
| | | -- -- |
| | | B5 |
| | 201 | PEETNYKAVS YHASGHSVAY KPGGFKASTG FGSNTKNKKI YDGGARTEDE |
| | 251 | VQSYPSKHDY V |

In accordance with one embodiment of the present disclosure, provided is an antibody or fragment thereof having binding specificity to a wild-type human claudin 18.2 (CLDN18.2) protein, wherein the antibody or the fragment thereof binds to both the first extracellular loop and the second extracellular loop of CLDN18.2. In some embodiments, the antibody or the fragment thereof binds to both the β3-β4 loop and the β5 strand of CLDN18.2.

In accordance with another embodiment of the present disclosure, provided is an antibody or fragment thereof having binding specificity to a wild-type human claudin 18.2 (CLDN18.2) protein, wherein the antibody or fragment further binds to a M149L mutant of the CLDN18.2 protein. In some embodiments, the antibody or fragment does not bind to a human wild-type claudin 18.1 (CLDN18.1) protein, or does not bind CLDN18.1 at an affinity that is greater than about 1% of the affinity to the wild-type CLDN18.2 protein.

The binding affinity of an antibody or fragment to a protein can be measured with many methods known in the art. For examples, it can be measured cell-free assays with standalone CLDN18.1 or CLDN18.2 proteins. Preferably, however, the measurement is done with the CLDN18.1 or CLDN18.2 protein on a cell surface mimicking the actual binding environment. Such binding assays are adequately exemplified in the experimental examples.

In some embodiments, the antibodies or fragments thereof have a binding affinity to the M149L mutant that is at least 1%, or alternatively at least 0.001%, 0.01%, 0.1%, 0.5%, 2%, 3%, 5%, 10%, 15%, 20%, 25%, 30%, 35%, 40%, 45%, 50%, 60%, 70%, 80%, 90%, 95%, 98%, or 99% of the affinity to the wild-type CLDN18.2 protein.

In some embodiments, the antibodies or fragments thereof do not bind human CLDN18.1. In some embodiments, the antibodies or fragments thereof, as compared to binding to CLDN18.2, have a much weaker binding to human CLDN18.1, e.g., not greater than 10%, 5%, 2%, 1%, 0.5%, 0.1%, 0.05%, 0.01%, 0.005%, or 0.001%, without limitation.

As described above, the antibodies and fragments thereof of the present disclosure bind to the claudin 18.2 protein at an epitope that is different from known antibodies (see FIG. 4; at least the reference antibody interacts with M149 whereas the presently disclosed ones do not). In one embodiment, therefore, provided is an antibody or fragment thereof having binding specificity to a wild-type human claudin 18.2 (CLDN18.2) protein, wherein the binding between the antibody or fragment thereof and the wild-type CLDN18.2 protein involves amino acid residues comprising at least an amino acid residue selected from the group consisting of N45, Y46, G48, V54, R55, E56, S58, F60, and E62; and at least an amino acid residue selected from the group consisting of Y169 and G172, of the wild-type CLDN18.2 protein.

In some embodiments, the antibody or fragment thereof binds to N45 of CLDN18.2. In some embodiments, the antibody or fragment thereof binds to Y46 of CLDN18.2. In some embodiments, the antibody or fragment thereof binds to G48 of CLDN18.2. In some embodiments, the antibody or fragment thereof binds to L49 of CLDN18.2. In some embodiments, the antibody or fragment thereof binds to W50 of CLDN18.2. In some embodiments, the antibody or fragment thereof binds to C53 of CLDN18.2. In some embodiments, the antibody or fragment thereof binds to V54 of CLDN18.2. In some embodiments, the antibody or fragment thereof binds to R55 of CLDN18.2. In some embodiments, the antibody or fragment thereof binds to E56 of CLDN18.2. In some embodiments, the antibody or fragment thereof binds to E58 of CLDN18.2. In some embodiments, the antibody or fragment thereof binds to F60 of CLDN18.2. In some embodiments, the antibody or fragment thereof binds to E62 of CLDN18.2. In some embodiments, the antibody or fragment thereof binds to C63 of CLDN18.2.

In some embodiments, the antibody or fragment thereof binds to at least two amino acid residues selected from N45, Y46, G48, V54, R55, E56, S58, F60, and E62. In some embodiments, the antibody or fragment thereof binds to at least three amino acid residues selected from N45, Y46, G48, V54, R55, E56, S58, F60, and E62. In some embodiments, the antibody or fragment thereof binds to at least four amino acid residues selected from N45, Y46, G48, V54, R55, E56, S58, F60, and E62. In some embodiments, the antibody or fragment thereof binds to at least five amino acid residues selected from N45, Y46, G48, V54, R55, E56, S58, F60, and E62.

In some embodiments, the antibody or fragment thereof binds to at least Y169 of CLDN18.2. In some embodiments, the antibody or fragment thereof binds to at least G172 of CLDN18.2. In some embodiments, the antibody or fragment thereof binds to at least two amino acid residues selected from Y169 and G172 of CLDN18.2.

In some embodiments, the binding involves amino acid residues comprising W30; two, three, four, five or more amino acid residues selected from the group consisting of N45, Y46, G48, V54, R55, E56, S58, F60, and E62; and at least an amino acid residue selected from the group consisting of Y169 and G172, of the wild-type CLDN18.2 protein. In some embodiments, the binding involves amino acid residues comprising W30, N45, Y46, G48, V54, R55, E56, S58, F60, E62 and Y169 of the wild-type CLDN18.2 protein.

The weaker bindings to these amino acids on CLDN18.2 may be as compared to the other amino acids, such as G48, L49, W50, C53, V54, R55, E56. In some embodiments, the comparison is to the binding at the same amino acid to 175D10. For instance, the binding of the antibody or fragment of the present disclosure is weaker than that of 175D10 (IMGT/2Dstructure-DB card No: 10473) to at least one, two, three, four, five or all of D28, Q33, N38, V43, G59 and V79.

In some embodiments, the antibody or fragment thereof does not bind M149L of the CLDN18.2 protein. In some embodiments, the antibody or fragment thereof binds to a M149L mutant of the CLDN18.2 protein.

In accordance with one embodiment of the present disclosure, provided is an antibody or fragment thereof that includes the heavy chain and light chain variable domains with the CDR regions as shown in the CDR combinations of Table A.

TABLE A
CDR combinations of tested antibodies (Kabat numbering)

| Comb | | | CDRL2 | CDRL3 | CDRH1 | CDRH2 | CDRH3 |
|---|---|---|---|---|---|---|---|
| No. | Antibody | CDRL1 (SEQ ID NO:) | (SEQ ID NO:) | (SEQ ID NO:) | (SEQ ID NO:) | (SEQ ID NO:) | (SEQ ID NO:) |
| 1 | 64G11B4 | KSSQSLLNSGNORNYLT (208) | WASTRES (227) | QNDYFYPFT (42) | NYLLE (234) | EINPGNGGSNYNEKFKG (255) | IYYGNSFAY (281) |
| 2 | 65G8B8 | KSSQSLLNSGNLKNYLT (209) | WASTRES (227) | QNVYIYPFT (43) | SYGVS (235) | VIWGDGNTIYHSALKS (256) | QGLYGHAMDY (282) |
| 3 | 56E8F10F4 | KSSQSLLNSGNQKNYLT (210) | WASTRES (227) | QNDYYFPFT (44) | SFGMN (236) | FISGGSNTIHYLDTVKG (257) | LALGNAMDY (283) |
| 4 | 54A2C4 | KSSQSLLNGGNQKNYLA (211) | GASTRES (228) | QNDLYYPWT (45) | TNAMN (237) | RIRSKSNNYATYYADSVKD (258) | GAYYGNSKAFDY (284) |
| 5 | 54A2C4′ | KSSQSLLNGGNQKNYLA (211) | GASTRES (228) | QNDLYYPWT (45) | NYLLE (234) | EINPGNGGSNYNEKFKG (255) | IYYGNSFAY (281) |
| 6 | 54A2C4″ | KSSQSLLNGGNQKNYLA (211) | GASTRES (228) | QNDLYYPWT (45) | TYSIH (238) | YINPSTIYTNYNQKFKY (259) | EGYGRGNAMDY (285) |
| 7 | 44F6B11 | KSNQSLLNSGNOKKYLT (212) | WASTRES (227) | QNGYSYPFT (46) | NYGMS (239) | TFSYGDSHNYYSDSVKG (260) | FGRGNTMDY (286) |
| 8 | 15C2B7 | KSSQSLLNSGNQKNYLT (210) | WASTRES (227) | QNNYYFPLT (47) | NYGMN (240) | WINANTGEPTYAEEFKG (261) | LTRGNSFDY (287) |
| 9 | 20F1E10 | KSSQSLFNSGNORNYLT (213) | WASTRES (227) | QNVYSYPLT (48) | KYGMN (241) | WISTNTGEPTYAEEFKG (262) | LVRGNSFDF (288) |
| 10 | 72C1B6A3 | KSSQSLLNSGNQKNYLT (210) | RASSRES (229) | QNDYIYPYT (8) | TYPIE (242) | NFHPYNDDTKYNEKFKG (263) | RAYGYPYAMDY (289) |
| 11 | 58G2C2 | KSSQSLLNSGNQKNYLT (210) | WAFTRES (230) | QNSYSYPFT (49) | NYLIE (243) | VINPGRSGTNYNEKFKG (264) | TRYGGNAMDY (290) |
| 12 | 101C4F12 | KSSQSLLNSGNORNYLT (208) | WSSTRDS (231) | QNNFIYPLT (50) | SYGVH (244) | VIWAGGSTNYDSALMS (265) | SLYGNSLDS (291) |
| 13 | 103A10B2 | RSSMSLFNSGNQKSYLS (214) | WASTRDS (232) | HNDYIYPLT (51) | SFGVH (245) | VIWAGGSTNYNSALMS (266) | SLYGNSFDY (292) |
| 14 | 78E8G9G6 | RSIQSLLNSGNOKNYLS (215) | WASTRES (227) | QNSYSYPFT (49) | SYGVH (244) | VIWAGGRTNYNSALMS (267) | DRYGGNSLDY (293) |
| 15 | 4F11E2 | RSSQSLLNSGNRKNYLT (216) | WASTRES (227) | QNAYSYPFT (13) | TFGMH (246) | YITSGNSPIYFTDTVKG (268) | SSYYGNSMDY (294) |
| 16 | 10G7G11 | KSSQSLFNSGNORNYLT (213) | WASTRES (227) | QNAYYFPFT (19) | TYGVH (247) | VMLSDGNTVYNSSLKS (269) | HKAYGNAMDY (295) |
| 17 | 12F1F4 | KSSQSLENSGNQRNYLT (213) | WSSTRES (233) | QNNYYYPFT (52) | NYGVS (248) | VIWGDGNTNYQSALRS (270) | VGRGNAMDH (296) |
| 18 | 78C10B6G4 | KSSQSLLNSGNQKNYLT (210) | RASSRES (229) | QNDYIYPYT (8) | NYGVS (248) | VIRGDGNTNYQSALRS (271) | VGRGNAMDH (296) |
| 19 | 119G11D9 | RSTQSLFNSGNQKNYLT (217) | WASTRES (227) | QNAYYYPLT (53) | GFLMH (249) | YINPYNDGTKYSEKFKG (272) | LDYGNAMDY (297) |
| 20 | 113G12E5E6 | KPSQSLLNSGNQKNYLA (218) | WASTRES (227) | QNAYFYPCT (54) | KYGVH (250) | VIWTGGNTDYNPALIP (273) | NGYYGNAMDY (298) |
| 21 | 116A8B7 | RSTQSLFNSGNORNYLT (219) | WASTRES (227) | QNAYYYPLT (53) | GFLMH (249) | YINPYNDGTKYSEKFKG (272) | LDYGNAMDY (297) |
| 22 | 105F7G12 | KSSQSLLNSGNQKNYLA (220) | WASTRES (227) | QNAYFYPCT (54) | KYGVH (250) | VIWTGGNTDYNPALIP (273) | NGYYGNAMDY (298) |
| 23 | 84E9E12 | KSSQSVENSGNQKNYLT (221) | WASTRES (227) | QNDYYFPLT (55) | SGYFW (251) | YISYDGSNNYNPSLKN (274) | FRFFAY (299) |
| 24 | 103F4D4 | RSSQSLLNGGNQKNYLT (222) | WASTRES (227) | QNAYFYPFT (56) | TYSIH (238) | YINPSTIYTNYNQKFKY (259) | EGYGRGNAMDY (285) |
| 25 | 110C12B6 | RSTQSLFNSGNORNYLT (219) | WASTRES (227) | QNAYYYPLT (53) | GFLMH (249) | YINPYNDGTKYSEKFKG (272) | LDYGNAMDY (297) |
| 26 | 85H12E8 | KSSQSLLNSGNORNYLS (223) | WASTRES (227) | QNAYFYPFT (56) | NYGVS (248) | VIWAGGNTNYNSALMS (275) | HGYGKGNAMDN (300) |
| 27 | 103H2B4 | KSSQSLLNSGNQKNYLT (210) | WASTRES (227) | QNNYFYPLT (57) | NFLTH (252) | EINPINGRTYYNEKFKR (276) | IYYGNSMDY (301) |
| 28 | 103F6D3 | RSSQSLLNGGNQKNYLT (222) | WASTRES (227) | QNAYFYPFT (56) | TYSIH (238) | YINPNTIYTNYNQKFKY (277) | EGYGRGNAMDY (285) |
| 29 | 113E12F7 | KSSQSLFNSGNQKNYLT (224) | WASTRES (227) | QNNYIYPLA (58) | SYGVH (244) | VIWAGGSTNYDSALMS (265) | SLYGNSFDH (302) |
| 30 | 120B7B2 | KSSQSLLNSGNQKNYLT (210) | WASTRES (227) | QNGYYFPFT (3) | GYIIQ (253) | FINPYNDGTKYNEQFKG (278) | AYFGNSFAY (303) |
| 31 | 111B12D11 | RSSQSLFNSGNQRNYLT (225) | WASTRES (227) | QNNYIYPLA (58) | SYGVH (244) | VIWAGGSTNYDSTLMS (279) | SLYGNSFDH (302) |
| 32 | 111E7E2 | KSSQSLFNSGNQKNYLT (224) | WASTRES (227) | QNNYIYPLA (58) | SYGAH (254) | VIWAGGSTNYDSALMS (265) | SLYGNSFDH (302) |
| 33 | 100F4G12 | KSTQSLLNSGNORNYLT (226) | WASTRES (227) | QNAYYYPLT (53) | GFLMH (249) | YINPYNDGTKYSERFKG (280) | LDYGNAMDY (297) |

TABLE B
CDRs of 120B7B2 (Kabat numbering)

| CDR | Sequence (SEQ ID NO:) | De-Risked Versions (SEQ ID NO:) |
|---|---|---|
| CDRL1 | KSSQSLLNSGNQKNYLT (210) | KSSQSLLNAGNQKNYLT (304) |
| | | KSSQSLLESGNQKNYLT (305) |
| CDRL2 | WASTRES (227) | |
| CDRL3 | QNGYYFPFT (3) | QNAYYFPFT (19) |
| CDRH1 | GYIIQ (253) | QEGYYFPFT (20) |
| CDRH2 | FINPYNDGTKYNEQFKG (278) | FINPYNDDTKYNEQFKG (306) |
| CDRH3 | AYFGNSFAY (303) | AYFGNAFAY (307) |

TABLE C
CDRs of 72C1B6A3 (Kabat numbering)

| CDR | Sequence (SEQ ID NO:) | De-Risked Versions (SEQ ID NO:) |
|---|---|---|
| CDRL1 | KSSQSLLNSGNQKNYLT (210) | KSSQSLLNAGNQKNYLT (304) |
| | | KSSQSLLESGNQKNYLT (305) |
| CDRL2 | RASSRES (229) | |
| CDRL3 | QNDYIYPYT (8) | |
| CDRH1 | TYPIE (242) | |
| CDRH2 | NFHPYNDDTKYNEKFKG (263) | |
| CDRH3 | RAYGYPYAMDY (289) | |

TABLE D
CDRs of 4F11E2 (Kabat numbering)

| CDR | Sequence (SEQ ID NO:) | De-Risked Versions (SEQ ID NO:) |
|---|---|---|
| CDRL1 | RSSQSLLNSGNRKNYLT (216) | RSSQSLLESGNRKNYLT (308) |
| | | RSSQSLLNAGNRKNYLT (309) |
| CDRL2 | WASTRES (227) | |
| CDRL3 | QNAYSYPFT (13) | |
| CDRH1 | TFGMH (246) | |
| CDRH2 | YITSGNSPIYFTDTVKG (268) | YITSGQSPIYFTDTVKG (310) |
| | | YITSGESPIYFTDTVKG (311) |
| CDRH3 | SSYYGNSMDY (294) | SSYYGQSMDY (312) |
| | | SSYYGESMDY (313) |
| | | SSYYGNAMDY (314) |

The antibodies that contained these CDR regions, whether mouse, humanized or chimeric, had potent claudin 18.2 binding and inhibitory activities. As shown in Examples 11 and 12, certain residues within the CDR can be modified to retain or improve the property or reduce their potential to have post-translational modifications (PTMs). Such modified CDR can be referred to as affinity matured or de-risked CDRs.

Non-limiting examples of de-risked CDRs are provided in Tables B-D, in the third columns. Affinity matured ones can include those having one, two or three amino acid addition, deletion and/or substitutions. In some embodiments, the substitutions can be conservative substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

TABLE E
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

TABLE F
Conservative Amino Acid Substitutions

| For Amino Acid | Substitution With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

In one embodiment, therefore, provided is an antibody or fragment thereof having binding specificity to a wild-type human claudin 18.2 (CLDN18.2) protein, wherein the antibody or fragment thereof comprises a light chain variable region comprising light chain complementarity determining regions CDRL1, CDRL2, and CDRL3 and a heavy chain variable region comprising heavy chain complementarity determining regions CDRH1, CDRH2, and CDRH3, and wherein the CDRL1, CDRL2, CDRL3, CDRH1, CDRH2, and CDRH3 are selected from combinations 1-33 of Table A or each of the combinations 1-33 in which one or more of the CDRL1, CDRL2, CDRL3, CDRH1, CDRH2, and CDRH3 each includes one, two, or three amino acid addition, deletion, conservative amino acid substitution or the combinations thereof.

In some embodiments, an anti-CLDN18.2 antibody or fragment is provided that includes CDRL1, CDRL2, CDRL3, CDRH1, CDRH2, and CDRH3, each of which is selected from Table A or Tables B-D. For instance, provided is an antibody or fragment thereof having binding specificity to a wild-type human claudin 18.2 (CLDN18.2) protein, wherein the antibody or fragment thereof comprises a light chain variable region comprising light chain complementarity determining regions CDRL1, CDRL2, and CDRL3 and a heavy chain variable region comprising heavy chain complementarity determining regions CDRH1, CDRH2, and CDRH3, and wherein: the CDRL1 comprises an amino acid sequence selected from the group of SEQ ID NO:208-226, or comprises an amino acid sequence derived anyone of SEQ ID NO:208-226 by one, two, or three amino acid addition, deletion, amino acid substitution; the CDRL2 comprises an amino acid sequence selected from the group of SEQ ID NO:227-233, or comprises an amino acid sequence derived anyone of SEQ ID NO: 227-233 by an amino acid addition, deletion, amino acid substitution; the CDRL3 comprises an amino acid sequence selected from the group of SEQ ID NO:3, 8, 13, 19 and 42-58, or comprises an amino acid sequence derived anyone of SEQ ID NO: 3, 8, 13, 19 and 42-58 by one, two, or three amino acid addition, deletion, amino acid substitution; the CDRH1 comprises an amino acid sequence selected from the group of SEQ ID NO:234-254, or comprises an amino acid sequence derived anyone of SEQ ID NO:234-254 by one, two, or three amino acid addition, deletion, amino acid substitution; the CDRH2 comprises an amino acid sequence selected from the group of SEQ ID NO:255-280, or comprises an amino acid sequence derived anyone of SEQ ID NO:255-280 by one, two, or three amino acid addition, deletion, amino acid substitution; and the CDRH3 comprises an amino acid sequence selected from the group of SEQ ID NO:281-303, or comprises an amino acid sequence derived anyone of SEQ ID NO:281-303 by one, two, or three amino acid addition, deletion, amino acid substitution.

In some embodiments, the CDRL1 comprises an amino acid sequence selected from the group of SEQ ID NO:208-226, 304-305 and 308-309; the CDRL2 comprises an amino acid sequence selected from the group of SEQ ID NO:227-233; the CDRL3 comprises an amino acid sequence selected from the group of SEQ ID NO:3, 8, 13, 19, 20 and 42-58; the CDRH1 comprises an amino acid sequence selected from the group of SEQ ID NO:234-254; the CDRH2 comprises an amino acid sequence selected from the group of SEQ ID NO:255-280, 306, 310 and 311; and the CDRH3 comprises an amino acid sequence selected from the group of SEQ ID NO:281-303, 307, and 312-314.

The antibody 120B7B2 has been demonstrated to be potent inhibitor of claudin 18.2. Its CDR sequences, along with a few de-risked versions, are provided in Table B. In one embodiment, the present disclosure provides an antibody or fragment thereof having binding specificity to a wild-type human claudin 18.2 (CLDN18.2) protein, wherein the antibody or fragment thereof comprises a light chain variable region comprising light chain complementarity determining regions CDRL1, CDRL2, and CDRL3 and a heavy chain variable region comprising heavy chain complementarity determining regions CDRH1, CDRH2, and CDRH3, and wherein: the CDRL1 comprises the amino acid sequence of QSLLNSGNQKNY (SEQ ID NO:1), QSLLNAGNQKNY (SEQ ID NO:17) or QSLLESGNQKNY (SEQ ID NO:18) or an amino acid sequence having one, two or three amino acid substitution from SEQ ID NO:1, 17 or 18, the CDRL2 comprises the amino acid sequence of WAS (SEQ ID NO:2) or an amino acid sequence having one or two amino acid substitution from SEQ ID NO:2, the CDRL3 comprises the amino acid sequence of CONGYYFPFT (SEQ ID NO:3), QNAYYFPFT (SEQ ID NO:19) or QEGYYFPFT (SEQ ID NO: 20) or an amino acid sequence having one, two or three amino acid substitution from SEQ ID NO:3, 19 or 20, the CDRH1 comprises the amino acid sequence of GYTFTGYI (SEQ ID NO:4) or an amino acid sequence having one, two or three amino acid substitution from SEQ ID NO:4, the CDRH2 comprises the amino acid sequence of INPYNDGT (SEQ ID NO: 5) or INPYNDDT (SEQ ID NO:21) or an amino acid sequence having one, two or three amino acid substitution from SEQ ID NO:5 or 21, and the CDRH3 comprises the amino acid sequence of ARAYFGNSFAY (SEQ ID NO:6) or ARAYFGNAFAY (SEQ ID NO:22) or an amino acid sequence having one, two or three amino acid substitution from SEQ ID NO: 6 or 22.

It is interesting to note (see Table A) that the CDRs from different antibody share great homology. It is then contemplated that each corresponding CDR can be interchanged without greatly impacting the antibody or fragment's binding affinity or activity. Alternatively, each particular amino acid in a CDR can be substituted with another amino acid present in a corresponding CDR from a different antibody.

In some embodiments, an antibody or fragment thereof is provided having binding specificity to a wild-type human claudin 18.2 (CLDN18.2) protein. In some embodiments, the antibody or fragment thereof comprises a light chain variable region comprising light chain complementarity determining regions CDRL1, CDRL2, and CDRL3 and a heavy chain variable region comprising heavy chain complementarity determining regions CDRH1, CDRH2, and CDRH3, and wherein: the CDRL1 comprises the amino acid sequence of SEQ ID NO: 210, 304 or 305 or an amino acid sequence having one, two or three amino acid substitution from SEQ ID NO:210, 304 or 305, the CDRL2 comprises the amino acid sequence of SEQ ID NO:227 or an amino acid sequence having one or two amino acid substitution from SEQ ID NO:227, the CDRL3 comprises the amino acid sequence of SEQ ID NO: 3, 19 or 20 or an amino acid sequence having one, two or three amino acid substitution from SEQ ID NO:3, 19 or 20, the CDRH1 comprises the amino acid sequence of SEQ ID NO:253 or an amino acid sequence having one, two or three amino acid substitution from SEQ ID NO: 253, the CDRH2 comprises the amino acid sequence of SEQ ID NO:278 or 306 or an amino acid sequence having one, two or three amino acid substitution from SEQ ID NO: 278 or 306, and the CDRH3 comprises the amino acid sequence of SEQ ID NO:303 or 307 or an amino acid sequence having one, two or three amino acid substitution from SEQ ID NO: 303 or 307.

In some embodiments, the CDRL1 comprises the amino acid sequence of SEQ ID NO: 210, 304 or 305, the CDRL2 comprises the amino acid sequence of SEQ ID NO:227, the CDRL3 comprises the amino acid sequence of SEQ ID NO:3, 19 or 20, the CDRH1 comprises the amino acid sequence of SEQ ID NO:253, the CDRH2 comprises the amino acid sequence of SEQ ID NO:278 or 306, and the CDRH3 comprises the amino acid sequence of SEQ ID NO:303 or 307.

Non-limiting examples of a light chain variable region includes an amino acid sequence selected from the group consisting of SEQ ID NO:141, 192-195 and 206-207, or a biological equivalent, such as a peptide having at least 90% sequence identity to an amino acid sequence selected from the group consisting of SEQ ID NO:141, 192-195 and 206-207.

Non-limiting examples of a heavy chain variable region include an amino acid sequence selected from the group consisting of SEQ ID NO:171, 188-191 and 205, or a biological equivalent, such as a peptide having at least 90% sequence identity to an amino acid sequence selected from the group consisting of SEQ ID NO:171, 188-191 and 205.

In some embodiments, the CDRL1 comprises the amino acid sequence of SEQ ID NO: 304, the CDRL2 comprises the amino acid sequence of SEQ ID NO:227, the CDRL3 comprises the amino acid sequence of SEQ ID NO:19, the CDRH1 comprises the amino acid sequence of SEQ ID NO:253, the CDRH2 comprises the amino acid sequence of SEQ ID NO: 306, and the CDRH3 comprises the amino acid sequence of SEQ ID NO:307. A non-limiting example of the antibody or fragment includes a light chain variable region comprising an amino acid sequence of SEQ ID NO:206 and a heavy chain variable region comprising an amino acid sequence of SEQ ID NO:205.

Likewise, 72C1B6A3 has been shown to be a good antibody. In another embodiment, therefore, provided is an antibody or fragment thereof having binding specificity to a wild-type human claudin 18.2 (CLDN18.2) protein, wherein the antibody or fragment thereof comprises a light chain variable region comprising light chain complementarity determining regions CDRL1, CDRL2, and CDRL3 and a heavy chain variable region comprising heavy chain complementarity determining regions CDRH1, CDRH2, and CDRH3, and wherein: the CDRL1 comprises the amino acid sequence of SEQ ID NO:210, 304 or 305 or an amino acid sequence having one, two or three amino acid substitution from SEQ ID NO:210, 304 or 305, the CDRL2 comprises the amino acid sequence of SEQ ID NO:229 or an amino acid sequence having one or two amino acid substitution from SEQ ID NO:229, the CDRL3 comprises the amino acid sequence of SEQ ID NO:8 or an amino acid sequence having one, two or three amino acid substitution from SEQ ID NO:8, the CDRH1 comprises the amino acid sequence of SEQ ID NO:242 or an amino acid sequence having one, two or three amino acid substitution from SEQ ID NO:242, the CDRH2 comprises the amino acid sequence of SEQ ID NO:263 or an amino acid sequence having one, two or three amino acid substitution from SEQ ID NO:263, and the CDRH3 comprises the amino acid sequence of SEQ ID NO: 289 or an amino acid sequence having one, two or three amino acid substitution from SEQ ID NO:289.

In some embodiments, the CDRL1 comprises the amino acid sequence of SEQ ID NO: 210, 304 or 305, the CDRL2 comprises the amino acid sequence of SEQ ID NO:229, the CDRL3 comprises the amino acid sequence of SEQ ID NO:8, the CDRH1 comprises the amino acid sequence of SEQ ID NO:242, the CDRH2 comprises the amino acid sequence of SEQ ID NO:263, and the CDRH3 comprises the amino acid sequence of SEQ ID NO:289.

Non-limiting examples of a light chain variable region include an amino acid sequence selected from the group consisting of SEQ ID NO:124, 185-187 and 203-204, or a biological equivalent, such as a peptide having at least 90% sequence identity to an amino acid sequence selected from the group consisting of SEQ ID NO:124, 185-187 and 203-204.

Non-limiting examples of a heavy chain variable region comprising an amino acid sequence selected from the group consisting of SEQ ID NO:153 and 181-184, or a biological equivalent, such as a peptide having at least 90% sequence identity to an amino acid sequence selected from the group consisting of SEQ ID NO:153 and 181-184.

In some embodiments, the CDRL1 comprises the amino acid sequence of SEQ ID NO: 304, the CDRL2 comprises the amino acid sequence of SEQ ID NO:229, the CDRL3 comprises the amino acid sequence of SEQ ID NO:8, the CDRH1 comprises the amino acid sequence of SEQ ID NO:242, the CDRH2 comprises the amino acid sequence of SEQ ID NO: 263, and the CDRH3 comprises the amino acid sequence of SEQ ID NO:289. A non-limiting example antibody or fragment thereof includes a light chain variable region comprising an amino acid sequence of SEQ ID NO:203 and a heavy chain variable region comprising an amino acid sequence of SEQ ID NO:181.

Also, 4F11E2 has been shown to be a good antibody. In another embodiment, therefore, provided is an antibody or fragment thereof having binding specificity to a wild-type human claudin 18.2 (CLDN18.2) protein, wherein the antibody or fragment thereof comprises a light chain variable region comprising light chain complementarity determining regions CDRL1, CDRL2, and CDRL3 and a heavy chain variable region comprising heavy chain complementarity determining regions CDRH1, CDRH2, and CDRH3, and wherein: the CDRL1 comprises the amino acid sequence of SEQ ID NO:216, 308 or 309 or an amino acid sequence having one, two or three amino acid substitution from SEQ ID NO:216, 308 or 309, the CDRL2 comprises the amino acid sequence of SEQ ID NO:227 or an amino acid sequence having one or two amino acid substitution from SEQ ID NO:227, the CDRL3 comprises the amino acid sequence of SEQ ID NO:13 or an amino acid sequence having one, two or three amino acid substitution from SEQ ID NO:13, the CDRH1 comprises the amino acid sequence of SEQ ID NO:246 or an amino acid sequence having one, two or three amino acid substitution from SEQ ID NO:246, the CDRH2 comprises the amino acid sequence of SEQ ID NO:268, 310 or 311 or an amino acid sequence having one, two or three amino acid substitution from SEQ ID NO:268, 310 or 311, and the CDRH3 comprises the amino acid sequence of SEQ ID NO:294, 312, 313 or 314, or an amino acid sequence having one, two or three amino acid substitution from SEQ ID NO:294, 312, 313 or 314.

In some embodiments, the CDRL1 comprises the amino acid sequence of SEQ ID NO: 216, 308 or 309, the CDRL2 comprises the amino acid sequence of SEQ ID NO:227, the CDRL3 comprises the amino acid sequence of SEQ ID NO:13, the CDRH1 comprises the amino acid sequence of SEQ ID NO:246, the CDRH2 comprises the amino acid sequence of SEQ ID NO:268, 310 or 311, and the CDRH3 comprises the amino acid sequence of SEQ ID NO: 294, 312, 313 or 314.

Non-limiting examples of a light chain variable region include an amino acid sequence selected from the group consisting of SEQ ID NO:129, 178-180 and 201-202, or a biological equivalent, such as a peptide having at least 90% sequence identity to an amino acid sequence selected from the group consisting of SEQ ID NO:129, 178-180 and 201-202.

Non-limiting examples of a heavy chain variable region comprising an amino acid sequence selected from the group consisting of SEQ ID NO:159, 175-177 and 196-200, or a biological equivalents, such as a peptide having at least 90% sequence identity to an amino acid sequence selected from the group consisting of SEQ ID NO:159, 175-177 and 196-200.

In some embodiments, the CDRL1 comprises the amino acid sequence of SEQ ID NO: 309, the CDRL2 comprises the amino acid sequence of SEQ ID NO:227, the CDRL3 comprises the amino acid sequence of SEQ ID NO:13, the CDRH1 comprises the amino acid sequence of SEQ ID NO:246, the CDRH2 comprises the amino acid sequence of SEQ ID NO: 311, and the CDRH3 comprises the amino acid sequence of SEQ ID NO:294. A non-limiting example of the antibody or fragment includes a light chain variable region comprising an amino acid sequence of SEQ ID NO:202 and a heavy chain variable region comprising an amino acid sequence of SEQ ID NO:197.

In some embodiments, the antibodies are humanized antibodies. Humanized antibodies, as shown in Example 9, can include one or more back mutations to the mouse counterpart. Examples of such back mutations are shown in Table 3. In some embodiments, the antibody or fragment can include one, two, three, four, five or more of the back mutations.

In some embodiments, the anti-claudin 18.2 antibody of the present disclosure includes a VL of any one of SEQ ID NO: 117-144, 178-180, 185-187, 192-195, 201-202, 203-204 or 206-207, and a VH of any one of SEQ ID NO: 145-174, 175-177, 181-184, 188-191, 196-200, or 205 or their respective biological equivalents. A biological equivalent of a VH or VL is a sequence that includes the designated amino acids while having an overall 80%, 85%, 90%, 95%, 98% or 99% sequence identity. A biological equivalent of SEQ ID NO: 145, therefore, can be a VH that has an overall 80%, 85%, 90%, 95%, 98% or 99% sequence identity to SEQ ID NO:145 but retains the CDRs, and optionally retains one or more, or all of the back-mutations.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

In certain embodiments, the antibody comprises an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

### Antibody-Drug Conjugates

In some embodiments, the antibodies or fragments may be conjugated to therapeutic agents, prodrugs, peptides, proteins, enzymes, viruses, lipids, biological response modifiers, pharmaceutical agents, or PEG.

In one embodiment, the antibodies or fragments of the disclosure are covalently attached to a drug moiety. The drug moiety may be, or be modified to include, a group reactive with a conjugation point on the antibody. For example, a drug moiety can be attached by alkylation (e.g., at the epsilon-amino group lysines or the N-terminus of antibodies), reductive amination of oxidized carbohydrate, transesterification between hydroxyl and carboxyl groups, amidation at amino groups or carboxyl groups, and conjugation to thiols.

In some embodiments, the number of drug moieties, p, conjugated per antibody molecule ranges from an average of 1 to 8; 1 to 7, 1 to 6, 1 to 5, 1 to 4, 1 to 3, or 1 to 2. In some embodiments, p ranges from an average of 2 to 8, 2 to 7, 2 to 6, 2 to 5, 2 to 4 or 2 to 3. In other embodiments, p is an average of 1, 2, 3, 4, 5, 6, 7 or 8. In some embodiments, p ranges from an average of about 1 to about 20, about 1 to about 10, about 2 to about 10, about 2 to about 9, about 1 to about 8, about 1 to about 7, about 1 to about 6, about 1 to about 5, about 1 to about 4, about 1 to about 3, or about 1 to about 2. In some embodiments, p ranges from about 2 to about 8, about 2 to about 7, about 2 to about 6, about 2 to about 5, about 2 to about 4 or about 2 to about 3.

For example, when chemical activation of the protein results in formation of free thiol groups, the protein may be conjugated with a sulfhydryl reactive agent. In one aspect, the agent is one which is substantially specific for free thiol groups. Such agents include, for example, malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio.

The drug can be linked to the antibody or fragment by a linker. Suitable linkers include, for example, cleavable and non-cleavable linkers. A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (mc-Val-Cit-PABA) linker. Another linker is Sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Yet another linker is maleimidocaproyl (mc). Other suitable linkers include linkers hydrolyzable at a specific pH or a pH range, such as a hydrazone linker. Additional suitable cleavable linkers include disulfide linkers. The linker may be covalently bound to the antibody to such an extent that the antibody must be degraded intracellularly in order for the drug to be released e.g. the mc linker and the like.

A linker can include a group for linkage to the antibody. For example, linker can include an amino, hydroxyl, carboxyl or sulfhydryl reactive groups (e.g., malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio).

In some embodiments, the drug moiety is a cytotoxic or cytostatic agent, an immunosuppressive agent, a radioisotope, a toxin, or the like. The conjugate can be used for inhibiting the multiplication of a tumor cell or cancer cell, causing apoptosis in a tumor or cancer cell, or for treating cancer in a patient. The conjugate can be used accordingly in a variety of settings for the treatment of animal cancers. The conjugate can be used to deliver a drug to a tumor cell or cancer cell. Without being bound by theory, in some embodiments, the conjugate binds to or associates with a cancer cell expressing claudin 18.2, and the conjugate and/or drug can be taken up inside a tumor cell or cancer cell through receptor-mediated endocytosis.

Once inside the cell, one or more specific peptide sequences within the conjugate (e.g., in a linker) are hydrolytically cleaved by one or more tumor-cell or cancer-cell-associated proteases, resulting in release of the drug. The released drug is then free to migrate within the cell and induce cytotoxic or cytostatic or other activities. In some embodiments, the drug is cleaved from the antibody outside the tumor cell or cancer cell, and the drug subsequently penetrates the cell, or acts at the cell surface.

Examples of drug moieties or payloads are selected from the group consisting of DM1 (maytansine, N2′-deacetyl-N2′-(3-mercapto-1-oxopropyl)- or N2′-deacetyl-N2′-(3-mercapto-1-oxopropyl)-maytansine), mc-MMAD (6-maleimidocaproyl-monomethylauristatin-D or N-methyl-L-valyl-N-[(1S,2R)-2-methoxy-4-[(2S)-2-[(1R,2R)-1-methoxy-2-methyl-3-oxo-3-[(1S)-2-phenyl-1-(2-thiazolyl)ethyl]amino]propyl]-1-pyr rolidinyl]-1-[(1S)-1-methylpropyl]-4-oxobutyl]-N-methyl-(9Cl)-L-valinamide), mc-MMAF (maleimidocaproyl-monomethylauristatin For N-[6-(2,5-dihydro-2,5-dioxo-1H-pyrrol-1-yl)-1-oxohexyl]-N-methyl-L-valyl-L-valyl-(3R,4S,5S)-3-methoxy-5-methyl-4-(methylamino) heptanoyl-(αR, βR,2S)-β-methoxy-α-methyl-2-pyrrolidinepropanoyl-L-phenylalanine) and mc-Val-Cit-PABA-MMAE (6-maleimidocaproyl-ValcCit-(p-aminobenzyloxycarbonyl)-monomethylauristatin E or N-[[4-[N-[6-(2,5-dihydro-2,5-dioxo-1H-pyrrol-1-yl)-1-oxohexyl]-L-valyl-N5-(aminocarbonyl)-L-ornithyl]amino]phenyl]methoxy]carbonyl]-N-meth yl-L-valyl-N-[(1S,2R)-4-[(2S)-2-[(1R,2R)-3-[(1R,2S)-2-hydroxy-1-methyl-2-phenylethyl]amino]-1-methoxy-2-methyl-3-oxopropyl]-1-pyrrolidinyl]-2-methoxy-1-[(1S)-1-methylpropyl]-4-oxobutyl]-N-methyl-L-valinamide). DM1 is a derivative of the tubulin inhibitor maytansine while MMAD, MMAE, and MMAF are auristatin derivatives. In some embodiments, the drug moiety is selected from the group consisting of mc-MMAF and mc-Val-Cit-PABA-MMAE. In some embodiments, the drug moiety is a maytansinoid or an auristatin.

The antibodies or fragments may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The antibodies can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). Techniques for conjugating various moieties to an antibody are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al., (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. (52:119-58 (1982)).

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Treatment Methods

As described herein, the antibodies, variants, derivatives or antibody-drug conjugates of the present disclosure may be used in certain treatment and diagnostic methods.

The present disclosure is further directed to antibody-based therapies which involve administering the antibodies, fragments, or antibody-drug conjugates of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. As provided above, claudin 18.2 can be overexpressed in tumor cells, in particular gastric, pancreatic, esophageal, ovarian, and lung tumors. Inhibition of claudin 18.2 has been shown to be useful for treating the tumors.

Accordingly, in some embodiments, provided are methods for treating a cancer in a patient in need thereof. The method, in one embodiment, entails administering to the patient an effective amount of an antibody, fragment, or antibody-drug conjugate of the present disclosure. In some embodiments, at least one of the cancer cells (e.g., stromal cells) in the patient over-express claudin 18.2.

Cellular therapies, such as chimeric antigen receptor (CAR) T-cell therapies, are also provided in the present disclosure. A suitable cell can be used, that is put in contact with an anti-claudin 18.2 antibody of the present disclosure (or alternatively engineered to express an anti-claudin 18.2 antibody of the present disclosure). Upon such contact or engineering, the cell can then be introduced to a cancer patient in need of a treatment. The cancer patient may have a cancer of any of the types as disclosed herein. The cell (e.g., T cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, and lung cancers.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibodies, fragments, or antibody-drug conjugates or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the antibodies, fragments, or antibody-drug conjugates of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antibodies, fragments, or antibody-drug conjugates of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

### Compositions

The present disclosure also provides pharmaceutical compositions. Such compositions comprise an effective amount of an antibody, fragment, or antibody-drug conjugate, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1: Generation of Murine Monoclonal Antibodies Against Human Claudin 18 Isoform 2 (CLD 18A2)

a. Immunizations:

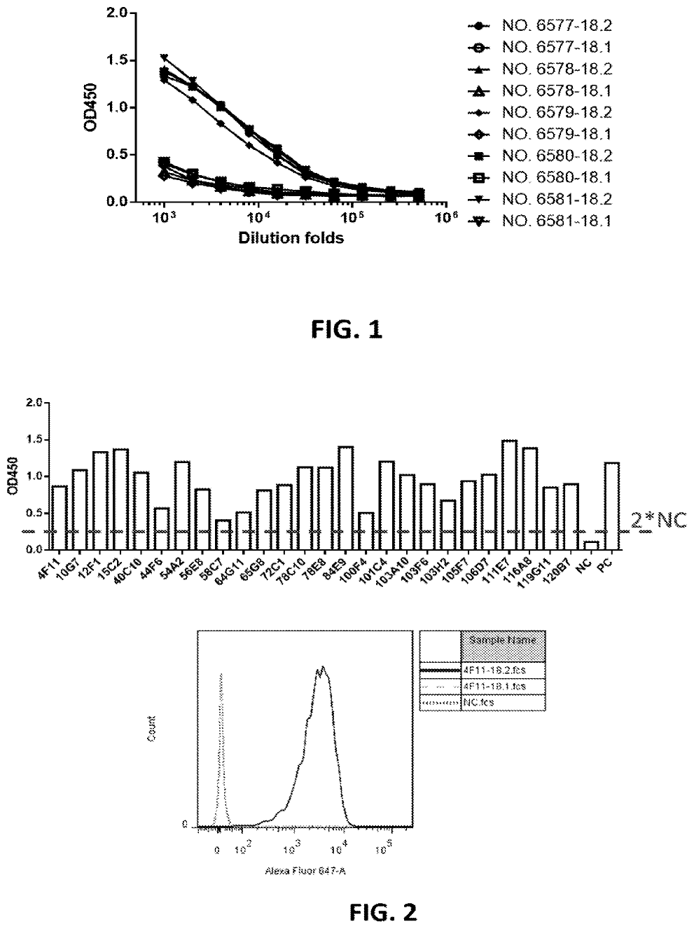

Balb/c and C57/BL6 mice were immunized with eukaryotic expression vectors, encoding human claudin 18.2 (CLD 18A2) fragments. 50 μg of plasmid DNA was injected into the quadriceps (intramuscular, i.m.) on day 1 and 10. The presence of antibodies directed against human CLD 18A2 in the serum of the mice was monitored on day 20 by flow cytometry, using HEK293 cells transiently transfected with a nucleic acid encoding human CLD 18A2. Mice with detectable immune responses (FIG. 1) were boosted three and two days prior to fusion by intraperitoneal injection of 5×10⁷ HEK293 cells transiently transfected with a nucleic acid encoding human CLD 18A2.

b. Generation of Hybridomas Producing Human Monoclonal Antibodies to CLD18A2:

Mouse splenocytes were isolated and fused with PEG to a mouse myeloma cell line based on standard protocols. The resulting hybridomas were then screened for production of immunoglobulins with CLD 18A2 specificity using HEK293 cells transfected with a nucleic acid encoding human CLD18 by cell ELISA.

Single cell suspensions of splenic lymphocytes from immunized mice were fused with P3X63Ag8U.1 non-secreting mouse myeloma cells (ATCC, CRL 1597) in a 2:1 ratio using 50% PEG (Roche Diagnostics, CRL 738641). Cells were plated at approximately 3×10⁴/well in flat bottom microtiter plates, followed by about two weeks incubation in selective medium containing 10% fetal bovine serum, 2% hybridoma fusion and cloning supplement (HFCS, Roche Diagnostics, CRL 1 363 735) plus 10 mM HEPES, 0.055 mM 2-mercaptoethanol, 50 μg/ml gentamycin and 1×HAT (Sigma, CRL H0262). After 10 to 14 days individual wells were screened by Cell ELISA for anti-CLD 18A2 monoclonal antibodies (FIG. 2). The antibody secreting hybridomas were re-plated, screened again with HEK293 expressing CLD 18A2 or CLD 18A1 by FACS and, if still positive for CLD18A2 and negative for CLD18A1, were subcloned by limiting dilution. The stable subclones were then cultured in vitro to generate small amounts of antibody in tissue culture medium for characterization. At least one clone from each hybridoma, which retained the reactivity of parent cells (by FACS), was chosen. Three vial cell banks were generated for each clone and stored in liquid nitrogen.

c. Selection of Monoclonal Antibodies Binding to CLD 18A2 not to CLD18A1:

To determine the isotype of antibodies, an isotype ELISA was performed. The mouse monoAB ID Kit (Zymed, CRL 90-6550) was used to determine Ig subclasses of the identified CLD18A2 reactive monoclonal antibodies. Thirty-two hybridoma cell lines were generated: 64G11B4, 65G8B8, 56E8F10F4, 54A2C4, 44F6B11, 15C2B7, 20F1E10, 72C1B6A3, 58G2C2, 101C4F12, 103A10B2, 40C10E3, 78E8G9G6, 4F11E2, 10G7G11, 12F1F4, 78C10B6G4, 119G11D9, 113G12E5E6, 116A8B7, 105F7G12, 84E9E12, 103F4D4, 110C12B6, 85H12E8, 103H2B4, 103F6D3, 113E12F7, 120B7B2, 111B12D11, 111E7E2, and 100F4G12, with further details as shown below:
64G11B4, mouse monoclonal IgG1, κ antibody
65G8B8, mouse monoclonal IgG1, κ antibody
56E8F10F4, mouse monoclonal IgG1, κ antibody
54A2C4, mouse monoclonal IgG1, κ antibody
44F6B11, mouse monoclonal IgG1, κ antibody
15C2B7, mouse monoclonal IgG1, κ antibody
20F1E10, mouse monoclonal IgG1, κ antibody
72C1B6A3, mouse monoclonal IgG1, κ antibody
58G2C2, mouse monoclonal IgG2a, κ antibody
101C4F12, mouse monoclonal IgG2b, κ antibody
103A10B2, mouse monoclonal IgG2b, κ antibody
40C10E3, mouse monoclonal IgG1, λ antibody
78E8G9G6, mouse monoclonal IgG1, κ antibody
4F11E2, mouse monoclonal IgG1, κ antibody
10G7G11, mouse monoclonal IgG1, κ antibody
12F1F4, mouse monoclonal IgG1, κ antibody
78C10B6G4, mouse monoclonal IgG1, κ antibody
119G11D9, mouse monoclonal IgG1, κ antibody
113G12E5E6, mouse monoclonal IgG1, κ antibody
116A8B7, mouse monoclonal IgG1, κ antibody
105F7G12, mouse monoclonal IgG1, κ antibody
84E9E12, mouse monoclonal IgG1, κ antibody
103F4D4, mouse monoclonal IgG1, κ antibody
110C12B6, mouse monoclonal IgG1, κ antibody
85H12E8, mouse monoclonal IgG1, κ antibody
103H2B4, mouse monoclonal IgG1, κ antibody
103F6D3, mouse monoclonal IgG1, κ antibody
113E12F7, mouse monoclonal IgG2a, κ antibody
120B7B2, mouse monoclonal IgG2a, κ antibody
111B12D11, mouse monoclonal IgG2a, κ antibody
111E7E2, mouse monoclonal IgG2a, κ antibody
100F4G12, mouse monoclonal IgG3, κ antibody.

### Example 2. Hybridoma Sequencing

Hybridoma cells (1×10⁷) were harvested and total RNA was extracted using Tri Reagent as described above for spleen tissue. cDNA was prepared using SuperScript III kit according to the manufacturer's instruction, described above. The resulting cDNA product was used as template for PCR with primers VhRevU and VhForU, the resulting 300 bp PCR product was cleaned up using a PCR clean-up kit and sequenced with the same primer. PCR reaction was also performed with light chain V-region specific primer VkRev7 and VkFor (for variable region only) or KappaFor primers (for entire kappa light chain). Sequencing reactions were performed on cleaned PCR product to obtain DNA sequences for the antibodies, 64G11B4, 65G8B8, 56E8F10F4, 54A2C4, 44F6B11, 15C2B7, 20F1E10, 72C1B6A3, 58G2C2, 101C4F12, 103A10B2, 40C10E3, 78E8G9G6, 4F11E2, 10G7G11, 12F1F4, 78C10B6G4, 119G11D9, 113G12E5E6, 116A8B7, 105F7G12, 84E9E12, 103F4D4, 110C12B6, 85H12E8, 103H2B4, 103F6D3, 113E12F7, 120B7B2, 111B12D11, 111E7E2, and 100F4G12. Their variable (VH and VL) sequences are shown in Table 1 below.

TABLE 1
Sequences of the variable regions of the antibodies

| Antibody | | SEQ |
|---|---|---|
| chain | Sequence | ID NO: |
| Light chains | | |
| 64G11B4 | DIVMTQSPSSLTVTAGEKVTMNCKSSQSLLNSGNQRNYLTWYQQKPGQPP | 117 |
| | KLLIYWASTRESGVPDRFTGSGSGTDFTLTISSVQADDLAVYYCQNDYFY | |
| | PFTFGAGTNLELK | |
| 65G8B8 | DIMMTQSPSSLTVTTGEKVTLTCKSSQSLLNSGNLKNYLTWYQQKPGHPP | 118 |
| | KLLIYWASTRESGVPVRFTGSGSGTDFTLTISSVQAEDLTVYYCQNVYIY | |
| | PFTFGSGTKLEMR | |
| 56E8F10F4 | DIVMTQSPSSLTVTAGEKVTMSCKSSQSLLNSGNQKNYLTWYQQKPGQPP | 119 |
| | KLLIYWASTRESGVPDRFTGSGSGTYFTLTISSVQAADLAVYYCQNDYYF | |
| | PFTFGSGTKLEIK | |
| 54A2C4 | DTVMTQFPSSLSVSAGEKVTMSCKSSQSLLNGGNQKNYLAWYQQKPGQPP | 120 |
| | KLLIYGASTRESGVPDRFTGSGSGTDFTLTISSVQAEDLAVYYCQNDLYY | |
| | PWTFGGGTKLEFK | |
| 44F6B11 | DIVMTQSPSSLTVTAGEKVIMSCKSNQSLLNSGNQKKYLTWYQQKPGQSP | 121 |
| | KLLIYWASTRESGVPDRFTGSESGTDFTLTISSVRAEDLAVYYCQNGYSY | |
| | PFTFGSGTKLEMK | |
| 15C2B7 | DIVMTQSPSSLTVTAGGKVTVSCKSSQSLLNSGNQKNYLTWYQQKPGQPP | 122 |
| | KLLIYWASTRESGVPDRFTGSGSGTDFTLTISSVQTEDLAVYYCQNNYYF | |
| | PLTFGAGTKLELK | |
| 20F1E10 | DIVMTQSPSSLTVTAGEKVTMSCKSSQSLFNSGNQRNYLTWYQQKPGQPP | 123 |
| | KLLIYWASTRESGVPDRFTGSGSGTDFILTITKVQAEDLAVYYCQNVYSY | |
| | PLTFGAGTKLELK | |
| 72C1B6A3 | DIVMTQSPSSLTVTAGEKVTMSCKSSQSLLNSGNQKNYLTWYQQRPGQPP | 124 |
| | KLLIYRASSRESGVPVRFTGSGSGTDFTLTISSVQAEDLAVYYCQNDYIY | |
| | PYTFGGGTKLEMN | |
| 58G2C2 | DIVMTQSPSSLTVTAGEKVTMSCKSSQSLLNSGNQKNYLTWYQQKPGQPP | 125 |
| | TLLIFWAFTRESGVPDRFTGSGSGTDFTLTINSVQAEDLAVYYCQNSYSY | |
| | PFTFGSGTKLEIK | |
| 101C4F12 | DIVMTQSPSSLTVTAGEKVTMSCKSSQSLLNSGNQRNYLTWYQQKPGQPP | 126 |
| | RLLIYWSSTRDSGVPDRFTGSGSRTDFTLTISSVQAEDLAVYYCQNNFIY | |
| | PLTFGAGTKLELK | |
| 103B2 | DIVMTQSPSSLTVTPGEKVTMSCRSSMSLFNSGNQKSYLSWYHQKPGQPP | 127 |
| | KLLIYWASTRDSGVPVRFTGSGSGTDFTLTISSVQAEDLAVYYCHNDYIY | |
| | PLTFGAGTKLELK | |
| 78E8G9G6 | DIVMTQSPSSLTVTAGEKVTMNCRSIQSLLNSGNQKNYLSWYQQKPGQPP | 128 |
| | KLLIYWASTRESGVPDRFTGSGSGTDFTLTIRSVLDEDLAVYYCQNSYSY | |
| | PFTFGSGTKLEMK | |
| 4F11E2 | DIVMTQSPSSLTVTAGEKVTLTCRSSQSLLNSGNRKNYLTWYQQIPGQPP | 129 |
| | KLLIYWASTRESGVPDRFTGSGSGTYFTLTISSVQAEDLAVYYCQNAYSY | |
| | PFTFGSGTKLEKK | |
| 10G7G11 | DIVMTQSPSSLTVTAGEKVTMTCKSSQSLFNSGNQRNYLTWYQRKPGQPP | 130 |
| | KLLIYWASTRESGVPDRFTGSGSGTYFTLTVSSVQAEDLAVYYCQNAYYF | |
| | PFTFGSGTKLEKK | |
| 12F1F4 | DIVMTQSPSSLTVTARERVSMTCKSSQSLENSGNQRNYLTWYQQKPGQPP | 131 |
| | KLLIYWSSTRESGVPDRFTGSGSGTDFTLTISSVQAEDLAIYFCQNNYYY | |
| | PFTFGSGTKLEIK | |
| 78C10B6G4 | DIVMTQSPSSLTVTAGEKVTMSCKSSQSLLNSGNQKNYLTWYQQRPGQPP | 124 |
| | KLLIYRASSRESGVPVRFTGSGSGTDFTLTISSVQAEDLAVYYCQNDYIY | |
| | PYTFGGGTKLEMN | |
| 119G11D9 | DIVMTQSPSSLTVTAGERVTMRCRSTQSLFNSGNQKNYLTWYQQKPGQPP | 132 |
| | KLLIYWASTRESGVPDRFTGGGSGTDFTLTISSVQAEDLAVYYCQNAYYY | |
| | PLTFGAGTKLERK | |
| 113G12E5E6 | DIVMTQSPSSLTVTAGERVTMSCKPSQSLLNSGNQKNYLAWYQQKPGQPP | 133 |
| | KLLLYWASTRESGVPDRFKGSGSGTDFTLTISSVQAEDLAVYYCQNAYFY | |
| | PCTFGGGTKLEMK | |
| 116A8B7 | DIVMTQSPSSLTVTAGEKVTMRCRSTQSLFNSGNQRNYLTWYQQKPGQPP | 134 |
| | KLLIYWASTRESGVPDRFTGSGSGTDFTLTISSVQAEDLAVYYCQNAYYY | |
| | PLTFGVGTKLERK | |
| 105F7G12 | DIVMTQSPSSLTVTAGERVTMSCKSSQSLLNSGNQKNYLAWYQQKPGQPP | 135 |
| | KLLLYWASTRESGVPDRFKGSGSGTDFTLTISSVQAEDLAVYYCQNAYFY | |
| | PCTFGGGTKLEMK | |
| 84E9E12 | DIVMTQSPSSLTVTTGEKVTMSCKSSQSVENSGNQKNYLTWYQQKPGQPP | 136 |
| | KLLVYWASTRESGVPARFTGSGSGTVFTLTISSVQAEDLAVYYCQNDYYF | |
| | PLTFGAGTRLELK | |
| 103F4D4 | DIVMTQSPSSQTVTAGEKVTLSCRSSQSLLNGGNQKNYLTWYQQKPGQPP | 137 |
| | KLLIYWASTRESGVPDRFTGSGSGTYFTFTISSVQAEDLAVYYCQNAYFY | |
| | PFTFGAGTKLELK | |
| 110C12B6 | DIVMTQSPSSLTVTAGEKVTMRCRSTQSLFNSGNQRNYLTWYQQKPGQPP | 134 |
| | KLLIYWASTRESGVPDRFTGSGSGTDFTLTISSVQAEDLAVYYCQNAYYY | |
| | PLTFGVGTKLERK | |
| 85H12E8 | DIVMTQSPSSLTVTAGEKVTMNCKSSQSLLNSGNQRNYLSWYQQEPGQPP | 138 |
| | KLLIYWASTRESGVPDRFTGSGSGTDFTLTISNIQAEDLALYFCQNAYFY | |
| | PFTFGSGTKLEIK | |
| 103H2B4 | DILMTQSPSSLTVTAGEKVTMSCKSSQSLLNSGNQKNYLTWYQQKPGQSP | 139 |
| | KLLIYWASTRESGVPDRFTGSGSGTDFTLTISSVQAEDLAVYYCQNNYFY | |
| | PLTFGVGTKLELK | |
| 103F6D3 | DIVMTQSPSSQTVTAGEKVTLSCRSSQSLLNGGNQKNYLTWYQQKPGQPP | 137 |
| | KLLIYWASTRESGVPDRFTGSGSGTYFTFTISSVQAEDLAVYYCQNAYFY | |
| | PFTFGAGTKLELK | |
| 113E12F7 | DIVMTQSPSSLTVTTGEKVTMSCKSSQSLFNSGNQKNYLTWYQQKPGQPP | 140 |
| | KLLIYWASTRESGVPDRFTGSGSGTYFTLTISSVQAEDLAVYYCQNNYIY | |
| | PLAFGTGTKLELK | |
| 120B7B2 | DIVMTQSPSSLTVTAGEKVTMSCKSSQSLLNSGNQKNYLTWYQQRPGQPP | 141 |
| | KLLMYWASTRESGVPDRFTGSGSGTDFTLTISSGQAEDLAIYFCQNGYYF | |
| | PFTFGSGTKLETK | |
| 111B12D11 | DIVMTQSPSSLTVTAGEKVTMRCRSSQSLFNSGNQRNYLTWYQQKPGQPP | 142 |
| | KLLIYWASTRESGVPDRFTGSGSGTDFTLTISSVQAEDLAVYYCQNNYIY | |
| | PLAFGAGTKLELK | |
| 111E7E2 | DIVMTQSPSSLTVTAGEKVTMSCKSSQSLFNSGNQKNYLTWYQQKPGQPP | 143 |
| | KLLIYWASTRESGVPDRFTGSGSGTDFTLTISSVQAEDLAVYYCQNNYIY | |
| | PLAFGAGTKLELK | |
| 100F4G12 | DIVMTQSPSSLTVTAGEKVTMRCKSTQSLLNSGNQRNYLTWYQQKPGQSP | 144 |
| | KLLIYWASTRESGVPERFTGSGSGTDFTLTISSVQAEDLAVYYCQNAYYY | |
| | PLTFGPGTKLERK | |
| Heavy Chain | | |
| 64G11B4 | QVQLHQSGTELVRPGTSVKVSCKASGYAFTNYLLEWVKQRPGQGLEWIGE | 145 |
| | INPGNGGSNYNEKFKGKATLTADKSSSTAYMQLSSLTSVDSAVYFCARIY | |
| | YGNSFAYWGQGTLVTVSA | |
| 65G8B8 | QVQLKESGPGLVAPSQSLSITCTVSGFSLTSYGVSWVRQPPGKGLEWLGV | 146 |
| | IWGDGNTIYHSALKSRLSISRDNSKRQVFLKVNSLQIDDTATYYCAKQGL | |
| | YGHAMDYWGQGTSVIVSS | |
| 56E8F10F4 | DVQLVESGGGLVQPGGSRKLSCTASGFTFNSFGMNWVRQAPEKGLEWVAF | 147 |
| | ISGGSNTIHYLDTVKGRFTISRDNPKNTLFLQMTSLRSEDTAMYYCTRLA | |
| | LGNAMDYWGQGTSVIVSS | |
| 54A2C4 | EVQHVETGGGLVQPKGSLKLSCAASGFTFNTNAMNWVRQAPGKGLEWVAR | 148 |
| | IRSKSNNYATYYADSVKDRFTISRDDSQSMLYVQMNNLKTEDTAMYYCVS | |
| | GAYYGNSKAFDYWGQGTLVTVSA | |
| 54A2C4′ | QVQLHQSGTELVRPGTSVKVSCKASGYAFTNYLLEWVKQRPGQGLEWIGE | 145 |
| | INPGNGGSNYNEKFKGKATLTADKSSSTAYMQLSSLTSVDSAVYFCARIY | |
| | YGNSFAYWGQGTLVTVSA | |
| 54A2C4″ | QVQLQQSGAELARPGASVKMSCKASGYTFPTYSIHWLKQGPGQGLEWIGY | 149 |
| | INPSTIYTNYNQKFKYKATLTADKSSSTAYIQLSSLTSDDSAVYYCAREG | |
| | YGRGNAMDYWGQGTSVTVSS | |
| 44F6B11 | EVQLVESGGDLVKPGGSLKLSCAASGFTFSNYGMSWVRQTPDKRLEWVAT | 150 |
| | FSYGDSHNYYSDSVKGRFTISRDIAKDALYLQMSSLRSEDTAIYYCARFG | |
| | RGNTMDYWGQGTSVTVSL | |
| 15C2B7 | QIQLVQSGPELRKPGETVKISCKASGYTFTNYGMNWVKQAPGKGLKWMAW | 151 |
| | INANTGEPTYAEEFKGRFAFSLETSARSAYLQINSLKNEDTATYFCARLT | |
| | RGNSFDYWGQGTTLTVSS | |
| 20F1E10 | QIQLVQSGPELKKPGETVKISCKASGYTFTKYGMNWVRQAPGKGLKWMGW | 152 |
| | ISTNTGEPTYAEEFKGRFAFSLETSASTAFLQINNLKNEDTATYFCARLV | |
| | RGNSFDFWGQGITLTVSS | |
| 72C1B6A3 | QVQLQQSGGELVKPGASVKMSCKAFGYTFTTYPIEWMKQNHGKSLEWIGN | 153 |
| | FHPYNDDTKYNEKFKGKAKLTVEKSSSTVYLEVSRLTSDDSAVYYCARRA | |
| | YGYPYAMDYWGQGTSVTVSS | |
| 58G2C2 | QVHLQQSGAEVVRPGTSVKVSCKASGYAFTNYLIEWIKKRPGQGLEWVGV | 154 |
| | INPGRSGTNYNEKFKGKATLTADKSSSTAYMQLSSLTSDDSAVYFCARTR | |
| | YGGNAMDYWGQGTSVTVSS | |
| 101C4F12 | QVQLKESGPGQVAPSQSLSIACTVSGFSLSSYGVHWVRQPPGKGLEWLGV | 155 |
| | IWAGGSTNYDSALMSRLTISKDNSRTRVFLKMNSLQTDDTAIYYCARSLY | |
| | GNSLDSWGPGTTLTVSS | |
| 103B2 | QVQLKESGPGLVAPSQSLSITCTVSGLSLTSFGVHWIRQPPGKGLEWLGV | 156 |
| | IWAGGSTNYNSALMSRLSISKDNSKSQVYLKMHSLQTDDTAMYYCARSLY | |
| | GNSFDYWGQGTALTVSS | |
| 40C10E3 | QVQLKESGPGLVAPSQSLSITCTVSGFSLSSYGVNWVRQPPGKGLEWLAA | 157 |
| | IRSDGIITYNSVLKSRLRISKDNSKSQVFLKMNSLQTDDTAMFYCARWFR | |
| | GNVLDYWGQGTSVTVSS | |
| 78E8G9G6 | QVQLKESGPGLVAPSQSLSITCTVSGFSLISYGVHWVRQPPGKGLEWLGV | 158 |
| | IWAGGRTNYNSALMSRLSISKDNSKSQVFLKMNSLQTYDTAMYYCARDRY | |
| | GGNSLDYWGQGTSVTVSS | |
| 4F11E2 | DVQLVESGGGLVQPGGSRKLSCAASGFTFSTFGMHWVRQAPEKGLEWVAY | 159 |
| | ITSGNSPIYFTDTVKGRFTISRDNPKNTLFLQMTSLGSEDTAVYYCARSS | |
| | YYGNSMDYWGQGTSVTVSS | |
| 10G7G11 | QVQLKESGPGLVAPSQSLSITCTISGFSLNTYGVHWVRQPPGKGLEWLVV | 160 |
| | MLSDGNTVYNSSLKSRLSLTKDNSKSQLLLKMNSLQTDDTAIYYCARHKA | |
| | YGNAMDYWGQGTSVTVSS | |
| 12F1F4 | QVQLKESGPGLVAPSQSLSITCTVSGFSLINYGVSWVRQPPGKGLEWLGV | 161 |
| | IWGDGNTNYQSALRSRLSIRKDTSKSQVFLKLNSVHTDGTATYYCAKVGR | |
| | GNAMDHWGQGISVIVSS | |
| 78C10B6G4 | QVQLKESGPGLVAPSQSLSITCTVSGFSLINYGVSWVRQPPGKGLEWLGV | 162 |
| | IRGDGNTNYQSALRSRLSIRKDTSKSQVFLKLNSVHTDGTATYYCAKVGR | |
| | GNAMDHWGQGISVIVSS | |
| 119G11D9 | EVQLQQSGPELVKPGASVKMSCKASGYTFTGFLMHWVKQKPGQGLEWIGY | 163 |
| | INPYNDGTKYSEKFKGKATLTSDKSSSTAFMELSSLTSDDSAVYYCARLD | |
| | YGNAMDYWGQGTSVTVSS | |
| 113G12E5E6 | QVQLKQSGPGLVQPSQSLSITCTVSDFSLTKYGVHWFRQSPGKGLEWLGV | 164 |
| | IWTGGNTDYNPALIPRLSFRKDNSKSQVFFKMNSLQSSDTAVYYCARNGY | |
| | YGNAMDYWGQGTSVTVSS | |
| 116A8B7 | EVQLQQSGPELVKPGASVKMSCKSSGYTFTGFLMHWVKQKPGQGLEWIGY | 165 |
| | INPYNDGTKYSEKFKGKATLTSDKSSSTAYMELSSLTSDDSAVYYCGRLD | |
| | YGNAMDYWGQGTSVTVSS | |
| 105F7G12 | QVQLKQSGPGLVQPSQSLSITCTVSDFSLTKYGVHWFRQSPGKGLEWLGV | 16 |
| | IWTGGNTDYNPALIPRLSFRKDNSKSQVFFKMNSLQSSDTAVYYCARNGY | |
| | YGNAMDYWGQGTSVTVSS | |
| 84E9E12 | DVQLQESGPGLVKPSQSLSLSCSVTGYSITSGYFWTWFRQFPGNKLEWMG | 166 |
| | YISYDGSNNYNPSLKNRISITRDTSKNQFFLKLNSVTTEDTATYYCASFR | |
| | FFAYWGQGTLVTVSA | |
| 103F4D4 | QVQLQQSGAELARPGASVKMSCKASGYTFPTYSIHWLKQGPGQGLEWIGY | 149 |
| | INPSTIYTNYNQKFKYKATLTADKSSSTAYIQLSSLTSDDSAVYYCAREG | |
| | YGRGNAMDYWGQGTSVTVSS | |
| 110C12B6 | EVQLQQSGPELVKPGASVKMSCKSSGYTFTGFLMHWVKQKPGQGLEWIGY | 165 |
| | INPYNDGTKYSEKFKGKATLTSDKSSSTAYMELSSLTSDDSAVYYCGRLD | |
| | YGNAMDYWGQGTSVTVSS | |
| 85H12E8 | QVQLKESGPGLVAPSQSLSITCTVSGFSLSNYGVSWVRQPPGKGLEWLGV | 167 |
| | IWAGGNTNYNSALMSRLRISKDNSKSQVFLKMNSLQTDDTARYYCARHGY | |
| | GKGNAMDNWGQGTSVTVSS | |
| 103H2B4 | QVQLQQPGAEPVKPGASVKLSCKASGYSFTNFLTHWVRQRPGQGLEWIGE | 168 |
| | INPTNGRTYYNEKFKRKATLTVDKSSTTVYMQLSNLTPEDSAVFYCARIY | |
| | YGNSMDYWGQGTLVTVSA | |
| 103F6D3 | QVQLQQSGAELARPGASVKMSCKASGYTFPTYSIHWLKQGPGQGLEWIGY | 169 |
| | INPNTIYTNYNQKFKYKTTLTADKSSSTAYIQLSSLTSDDSAVYYCAREG | |
| | YGRGNAMDYWGQGTSVTVSS | |
| 113E12F7 | QVQLKESGPGLVAPSQSLSITCTVTGFSLSSYGVHWVRQPPGKGLEWLGV | 170 |
| | IWAGGSTNYDSALMSRLSISKDRSKSQVFLKMTSLQTDDTAMYYCARSLY | |
| | GNSFDHWGQGTTLTVSS | |
| 120B7B2 | EVQLQQSGPELVKPGASVKMSCKASGYTFTGYIIQWMKQKPGLGLEWIGF | 171 |
| | INPYNDGTKYNEQFKGKATLTSDKSSNAAYMELSSLTSEDSAVYYCARAY | |
| | FGNSFAYWGQGTLVTVSA | |
| 111B12D11 | QVQLKESGPGLVAPSQSLSITCTVSGFSLTSYGVHWVRQPPGKGLEWLGV | 172 |
| | IWAGGSTNYDSTLMSRLSISKDRSKSQVFLKMTSLQTDDTAMYYCARSLY | |
| | GNSFDHWGQGTTLTVSS | |
| 111E7E2 | QVQLKESGPGLVAPSQSLSITCTVSGFSLTSYGAHWVRQPPGKGLEWLGV | 173 |
| | IWAGGSTNYDSALMSRLSISKDRSKSQVFLKMTSLQTDDTAMYYCARSLY | |
| | GNSFDHWGQGTTLTVSS | |
| 100F4G12 | EVQLQQSGPELVKPGASVKMSCKASGYTFTGFLMHWVKQKPGQGLEWIGY | 174 |
| | INPYNDGTKYSERFKGKATLTSDKSSSTAYMELSSLTSDDSAVYYCARLD | |
| | YGNAMDYWGQGTSVTVSS | |

### Example 3. Production and Purification of Monoclonal Antibodies Reactive to CLD18A2

To produce mg amounts of antibody for functional characterization, hybridoma cells were seeded in dialysis based bioreactors (CELLine CL1000, Integra, Chur, CH) at 2×10⁶ cells/ml. Antibody-containing supernatant was harvested once weekly. Each mouse monoclonal antibody was purified using Melon Gel (Pierce, Rockford, USA) and concentrated by ammonium sulphate precipitation. Antibody concentration and purity was estimated by sodium dodecylsulphate gel electrophoresis and coomassie staining.

### Example 4. Binding of Murine Monoclonal Antibodies Reactive to CLD18A2

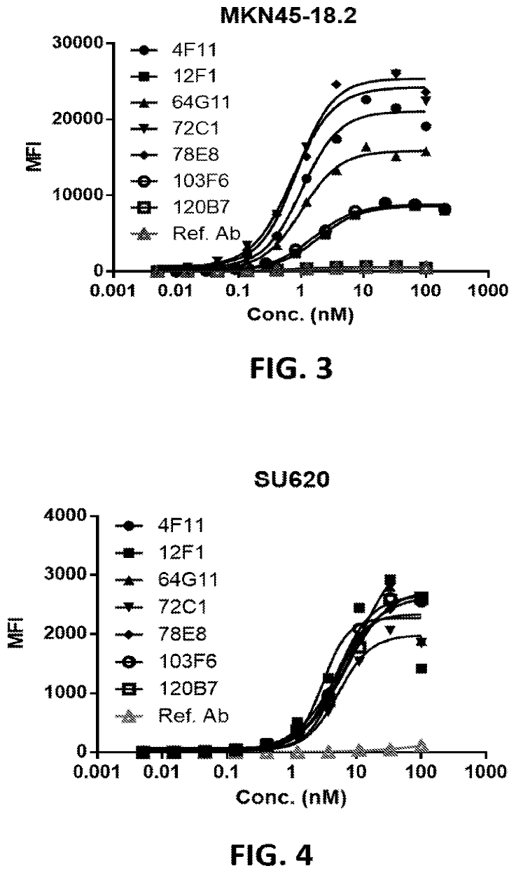

MKN45 cells that over-expressed CLD18A2 were harvested from flasks. 100 μl of 1×10⁶ cells/ml of cells were incubated with primary antibodies indicated as FIG. 3 in 3-fold serial dilutions starting from 100 nM to 0.003 nM for 30 minutes on ice. After being washed with 200 μl of FACS buffer twice, cells were incubated with secondary antibody for 30 minutes on ice. Cells were washed with 200 μl of FACS buffer twice and transferred to BD Falcon 5 ml tube and analyzed by FACS. The results of the study showed that the purified murine antibody could bind to MKN45 cells transfected with human CLD18A2 by flow cytometry with high EC50, compared with positive reference antibody.

### Example 5. Binding of Murine Monoclonal Antibodies Reactive to CLD18A2 Mutant

SU620 cells that endogenously expressed CLD18A2 bearing the M149L mutation were harvested from flasks. 100 μl of 1×10⁶ cells/ml of cells were incubated with primary antibodies indicated as FIG. 4 in 3-fold serial dilutions starting from 100 nM to 0.003 nM for 30 minutes on ice. After being washed with 200 μl of FACS buffer twice, cells were incubated with secondary antibody for 30 minutes on ice. Cells were washed with 200 μl of FACS buffer twice and transferred to BD Falcon 5 ml tube and analyzed by FACS. The results of the study showed that the purified murine antibody could bind to SU620 cells endogenously expressing human CLD18A2 bearing M149L mutation by flow cytometry with high EC50, while the refrence antibody did not (FIG. 4).

### Example 6. Binding of Murine Monoclonal Antibodies Reactive to Mouse and Cyno CLD18A2

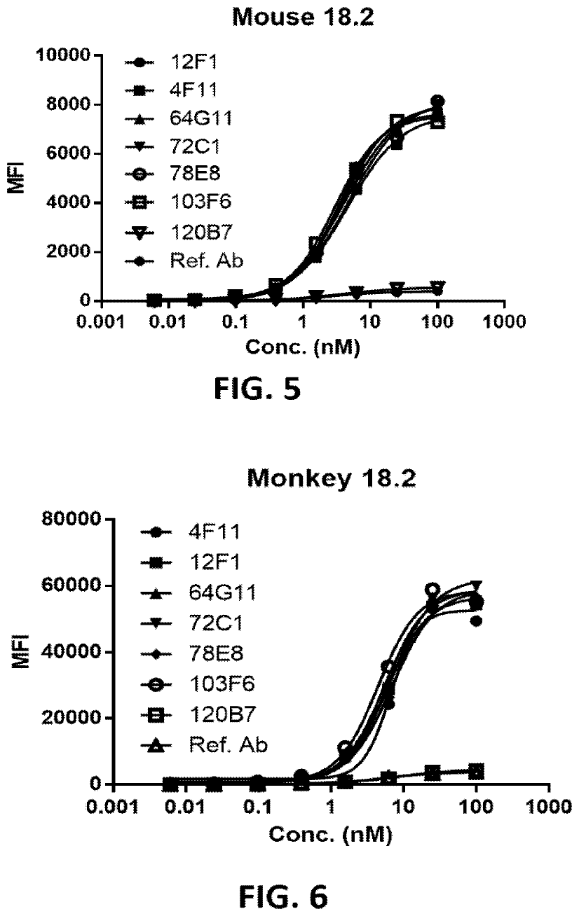

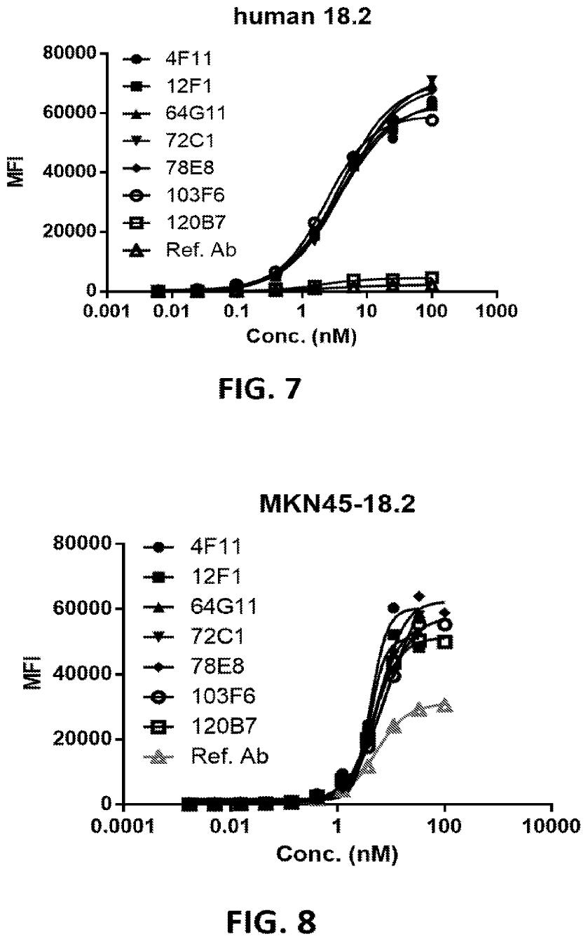

To evaluate these antibodies' cross-reactivities with mouse and cyno CLD18A2, HEK293 cells that over-expressed mouse, cyno or human CLD18A2 were harvested from flasks. 100 μl of 1×10⁶ cells/ml of cells were incubated with primary antibodies indicated as FIG. 3 in 3-fold serial dilutions starting from 100 nM to 0.003 nM for 30 minutes on ice. After being washed with 200 μl of FACS buffer twice, cells were incubated with secondary antibody for 30 minutes on ice. Cells were washed with 200 μl of FACS buffer twice and transferred to BD Falcon 5 ml tube and analyzed by FACS. The results of the study showed that the purified murine antibodies can bind to mouse and cyno CLD18A2 by flow cytometry with high EC50, at least similar to the reference anbibodies (FIGS. 5, 6 and 7).

### Example 7. Binding of Chimeric Antibodies Reactive to CLD18A2

The murine VH and VK genes were produced synthetically and then respectively cloned into vectors containing the human gamma 1 and human kappa constant domains. The purified chimeric antibodies were produced from transfected CHOs cells.

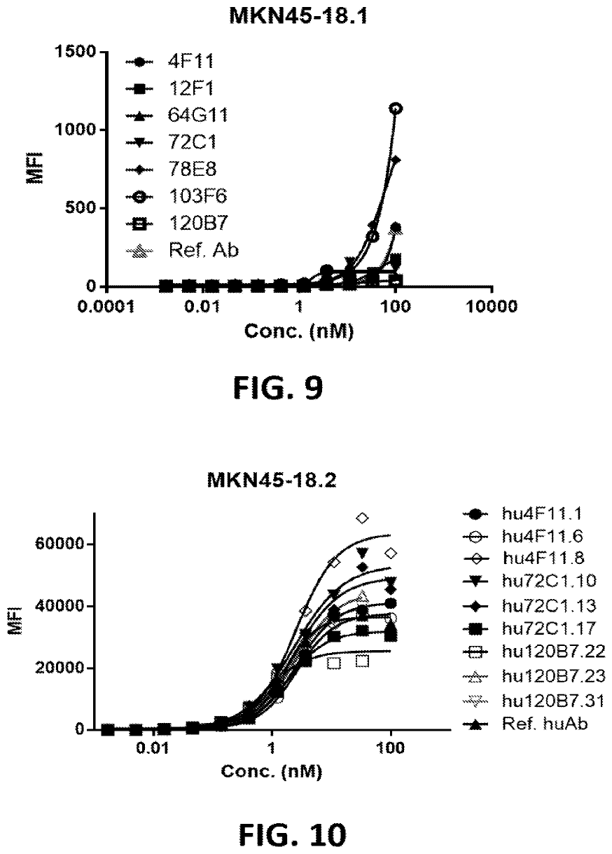

MKN45 cells that stably expressed human CLD18A2 or CLD18A1 were harvested from flasks. 100 μl of 1×10⁶ cells/ml of cells were incubated with primary chimeric antibodies indicated as FIG. 4 in 3-fold serial dilutions starting from 100 nM to 0.003 nM for 30 minutes on ice. After being washed with 200 μl of FACS buffer twice, cells were incubated with secondary antibody for 30 minutes on ice. Cells were washed with 200 μl of FACS buffer twice and transferred to BD Falcon 5 ml tube and analyzed by FACS. The results of the study showed that the chimeric antibodies can bind to human CLD18A2 with high EC50, but not CLD18A1 (FIGS. 8 and 9).

### Example 8. Antibody-Dependent Cellular Cytotoxicity (ADCC) of Chimeric Antibodies

The ADCC Reporter Bioassay uses an alternative readout at an earlier point in ADCC MOA pathway activation: the activation of gene transcription through the NFAT (nuclear factor of activated T-cells) pathway in the effector cell. In addition, the ADCC Reporter Bioassay uses engineered Jurkat cells stably expressing the FcγRIIIa receptor, V158 (high affinity) variant, and an NFAT response element driving expression of firefly luciferase as effector cells. Antibody biological activity in ADCC MOA is quantified through the luciferase produced as a result of NFAT pathway activation; luciferase activity in the effector cell was quantified with luminescence readout (FIG. 1). Signal was high, and assay background was low.

Serial dilutions of claudin 18.2 chimeric monoclonal antibody or Ref. Ab were incubated for 6 hours of induction at 37° C. with engineered Jurkat effector cells (ADCC Bioassay Effector Cells), with or without ADCC Bioassay Target Cells (expressing claudin 18.2). Luciferase activity was quantified using Bio-Glo™ Reagent (Table 2). The results show that these chimeric antibodies have very strong ADCC activities.

TABLE 2

| EC50 of the tested antibodies | |
|---|---|
| Antibody | EC50 (pM) |
| 4F11E2 | 22.18 |
| 12F1F4 | 36.77 |
| 64G11B4 | 125.7 |
| 72C186A3 | 46.32 |
| 78E8G9G6 | 15.86 |
| 103F6D3 | 79.53 |
| 120B7B2 | 5.806 |
| Ref. Ab | 458.5 |

### Example 9. Humanization of the 4F11E2, 72C1B6A3 and 120B7B2 Mouse mAbs

The mAb 4F11E2, 72C1B6A3 and 120B7B2 variable region genes were employed to create humanized MAbs. In the first step of this process, the amino acid sequences of the VH and VL of MAb were compared against the available database of human Ig gene sequences to find the overall best-matching human germline Ig gene sequences.

The amino acid sequences of the humanized antibodies are listed in Table 3 below.

TABLE 3
Humanized sequences

| | | SEQ ID |
|---|---|---|
| Name | Sequence | NO: |
| 4F11VH_1 | EVQLVESGGGLVQPGGSLRLSCAASGFTFSTFGMHWVRQAPGKGLEWVSY | 175 |
| (grafted VH) | ITSGNSPIYFTDTVKGRFTISRDNAKNSLYLQMNSLRAEDTAVYYCARSS | |
| | YYGNSMDYWGQGTLVTVSS | |
| 4F11VH_2 | EVQLVESGGGLVQPGGSLRLSCAASGFTFSTFGMHWVRQAPGKGLEWVAY | 176 |
| | ITSGNSPIYFTDTVKGRFTISRDNAKNSLYLQMNSLRAEDTAVYYCARSS | |
| | YYGNSMDYWGQGTLVTVSS | |
| 4F11VH_3 | EVQLVESGGGLVQPGGSLRLSCAASGFTFSTFGMHWVRQAPGKGLEWVAY | 177 |
| | ITSGNSPIYFTDTVKGRFTISRDNAKNTLYLQMNSLRAEDTAVYYCARSS | |
| | YYGNSMDYWGQGTLVTVSS | |
| 4F11VL_1 | DIVMTQSPDSLAVSLGERATINCRSSQSLLNSGNRKNYLTWYQQKPGQPP | 178 |
| (grafted VL) | KLLIYWASTRESGVPDRFSGSGSGTDFTLTISSLQAEDVAVYYCQNAYSY | |
| | PFTFGGGTKLEIK | |
| 4F11VL_2 | DTVMTQSPDSLAVSLGERATINCRSSQSLLNSGNRKNYLTWYQQKPGQPP | 179 |
| | KLLIYWASTRESGVPDRFSGSGSGTDFTLTISSVQAEDVAVYYCQNAYSY | |
| | PFTFGGGTKLEIK | |
| 4F11VL_3 | DTVMTQSPDSLAVSLGERVTLNCRSSQSLLNSGNRKNYLTWYQQKPGQPP | 180 |
| | KLLIYWASTRESGVPDRFSGSGSGTDFTLTISSVQAEDVAVYYCQNAYSY | |
| | PFTFGGGTKLEIK | |
| 72C1VH_1 | QVQLVQSGAEVKKPGASVKVSCKASGYTFTTYPIEWVRQAPGQRLEWMGN | 181 |
| (grafted VH) | FHPYNDDTKYNEKFKGRVTITRDTSASTAYMELSSLRSEDTAVYYCARRA | |
| | YGYPYAMDYWGQGTLVTVSS | |
| 72C1VH_2 | QVQLVQSGAEVVKPGASVKVSCKASGYTFTTYPIEWMRQAPGQRLEWMGN | 182 |
| | FHPYNDDTKYNEKFKGRVTITVDTSASTAYMELSSLRSEDTAVYYCARRA | |
| | YGYPYAMDYWGQGTLVTVSS | |
| 72C1VH_3 | QVQLVQSGAEVVKPGASVKVSCKASGYTFTTYPIEWMKQAPGQRLEWMGN | 183 |
| | FHPYNDDTKYNEKFKGRVTITVDTSASTAYMEVSSLRSEDTAVYYCARRA | |
| | YGYPYAMDYWGQGTLVTVSS | |
| 72C1VH_4 | QVQLVQSGAEVVKPGASVKMSCKASGYTFTTYPIEWMKQAPGQRLEWMGN | 184 |
| | FHPYNDDTKYNEKFKGRVTLTVDTSASTVYLEVSSLRSEDTAVYYCARRA | |
| | YGYPYAMDYWGQGTLVTVSS | |
| 72C1VL_1 | DIVMTQSPDSLAVSLGERATINCKSSQSLLNSGNQKNYLTWYQQKPGQPP | 185 |
| (grafted VL) | KLLIYRASSRESGVPDRFSGSGSGTDFTLTISSLQAEDVAVYYCQNDYIY | |
| | PYTFGGGTKLEIK | |
| 72C1VL_2 | DIVMTQSPDSLAVSLGERATISCKSSQSLLNSGNQKNYLTWYQQKPGQPP | 186 |
| | KLLIYRASSRESGVPDRFSGSGSGTDFTLTISSLQAEDVAVYYCQNDYIY | |
| | PYTFGGGTKLEIK | |
| 72C1VL_3 | DIVMTQSPDSLAVSLGERVTMSCKSSQSLLNSGNQKNYLTWYQQKPGQPP | 187 |
| | KLLIYRASSRESGVPDRFSGSGSGTDFTLTISSVQAEDVAVYYCQNDYIY | |
| | PYTFGGGTKLEIK | |
| 120B7VH_1 | QVQLVQSGAEVKKPGASVKVSCKASGYTFTGYIIQWVRQAPGQRLEWMGF | 188 |
| (grafted VH) | INPYNDGTKYNEQFKGRVTITRDTSASTAYMELSSLRSEDTAVYYCARAY | |
| | FGNSFAYWGQGTLVTVSS | |
| 120B7VH_2 | QVQLVQSGAEVVKPGASVKVSCKASGYTFTGYIIQWMRQAPGQRLEWMGF | 189 |
| | INPYNDGTKYNEQFKGRVTITSDTSASAAYMELSSLRSEDTAVYYCARAY | |
| | FGNSFAYWGQGTLVTVSS | |
| 120B7VH_3 | QVQLVQSGAEVVKPGASVKVSCKASGYTFTGYIIQWMKQAPGQRLEWIGF | 190 |
| | INPYNDGTKYNEQFKGRATITSDTSASAAYMELSSLRSEDTAVYYCARAY | |
| | FGNSFAYWGQGTLVTVSS | |
| 120B7VH_4 | EVQLVQSGAEVVKPGASVKMSCKASGYTFTGYIIQWMKQAPGQRLEWIGE | 191 |
| | INPYNDGTKYNEQFKGRATLTSDTSASAAYMELSSLRSEDTAVYYCARAY | |
| | FGNSFAYWGQGTLVTVSS | |
| 120B7VL_1 | DIVMTQSPDSLAVSLGERATINCKSSQSLLNSGNQKNYLTWYQQKPGQPP | 192 |
| (grafted VL) | KLLIYWASTRESGVPDRFSGSGSGTDFTLTISSLQAEDVAVYYCQNGYYF | |
| | PFTFGGGTKLEIK | |
| 120B7VL_2 | DIVMTQSPDSLAVSLGERATISCKSSQSLLNSGNQKNYLTWYQQKPGQPP | 193 |
| | KLLMYWASTRESGVPDRFSGSGSGTDFTLTISSLQAEDVAVYYCQNGYYF | |
| | PFTFGGGTKLEIK | |
| 120B7VL_3 | DIVMTQSPDSLAVSLGERATISCKSSQSLLNSGNQKNYLTWYQQKPGQPP | 194 |
| | KLLMYWASTRESGVPDRFSGSGSGTDFTLTISSGQAEDVAVYFCQNGYYF | |
| | PFTFGGGTKLEIK | |
| 120B7VL_4 | DIVMTQSPDSLAVSLGERVTMSCKSSQSLLNSGNQKNYLTWYQQKPGQPP | 195 |
| | KLLMYWASTRESGVPDRFSGSGSGTDFTLTISSGQAEDVAVYFCQNGYYF | |
| | PFTFGGGTKLEIK | |

The humanized VH and VL genes were produced synthetically and then respectively cloned into vectors containing the human gamma 1 and human kappa constant domains. The pairing of the human VH and the human VL created the humanized antibodies (see Table 4).

TABLE 4
Humanized antibodies with their VH an VL regions
4F11E2

| VH | | | |
|---|---|---|---|
| VL | 4F11VH_1 | 4F11VH_2 | 4F11VH_3 |
| 4F11VL_1 | hu4F11.1 | hu4F11.4 | hu4F11.7 |
| 4F11VL_2 | hu4F11.2 | hu4F11.5 | hu4F11.8 |
| 4F11VL_3 | hu4F11.3 | hu4F11.6 | hu4F11.9 |

72C1B6A3

| VH | | | | |
|---|---|---|---|---|
| VL | 72C1VH_1 | 72C1VH_2 | 72C1VH_3 | 72C1VH_4 |
| 72C1VL_1 | hu72C1.10 | hu72C1.13 | hu72C1.16 | hu72C1.19 |
| 72C1VL_2 | hu72C1.11 | hu72C1.14 | hu72C1.17 | hu72C1.20 |
| 72C1VL_3 | hu72C1.12 | hu72C1.15 | hu72C1.18 | hu72C1.21 |

120B7B2

| VH | | | | |
|---|---|---|---|---|
| VL | 120B7VH_1 | 120B7VH_2 | 120B7VH_3 | 120B7VH_4 |
| 120B7VL_1 | hu120B7.22 | hu120B7.26 | hu120B7.30 | hu120B7.34 |
| 120B7VL_2 | hu120B7.23 | hu120B7.27 | hu120B7.31 | hu120B7.35 |
| 120B7VL_3 | hu120B7.24 | hu120B7.28 | hu120B7.32 | hu120B7.36 |
| 120B7VL_4 | hu120B7.25 | hu120B7.29 | hu120B7.33 | hu120B7.37 |

### Example 10. Binding of Humanized Antibodies Reactive to CLD18A2

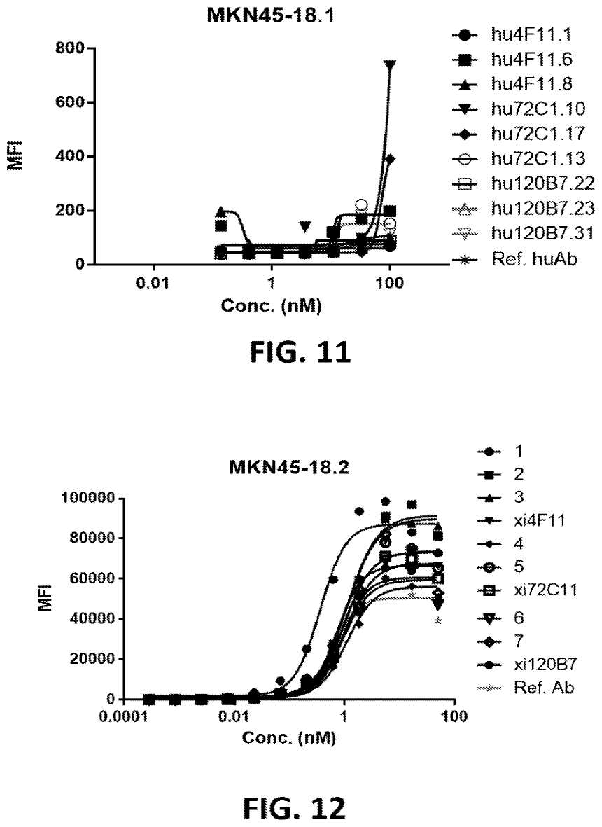

MKN45 cells that stably expressed human CLD18A2 or CLD18A1 were harvested from flasks. 100 μl of 1×10⁶ cells/ml of cells were incubated with primary humanized antibodies indicated as FIG. 4 in 3-fold serial dilutions starting from 100 nM to 0.003 nM for 30 minutes on ice. After being washed with 200 μl of FACS buffer twice, cells were incubated with secondary antibody for 30 minutes on ice. Cells were washed with 200 μl of FACS buffer twice and transferred to BD Falcon 5 ml tube and analyzed by FACS. The results of the study showed that the indicated humanized antibodies can bind to human CLD18A2 with high EC50, but not CLD18A1 (FIGS. 10 and 11).

### Example 11. Binding of PTM (Post-Translational Modification) De-Risk Humanized Antibodies Reactive to CLD18A2

Post-translational modifications (PTMs) can cause problems during the development of a therapeutic protein such as increased heterogeneity, reduced bioactivity, reduced stability, immunogenicity, fragmentation and aggregation. The potential impact of PTMs depends on their location and in some cases on solvent exposure. The CDRs of sequence were analyzed for the following potential PTMs: asparagine deamidation, aspartate isomerization, free cysteine thiol groups, N-glycosylation, oxidation, fragmentation by potential hydrolysis site etc.

To reduce the risk of developing PTM in 4F11E2, 72C1B6A3 and 120B7B2, some concerned amino acids in the VH and VL were mutated. And then nine antibodies were generated:

| Clones | HC:LC* | No. |
|---|---|---|
| 4F11E2 | HC N55Q-LC N31E | 1 |
| | HC N55Q-LC S32A | 2 |
| | HC N55E-LC S32A | 3 |
| | HC N55E&N104Q-LC S32A | 8 |
| | HC N55E&N104E-LC S32A | 9 |
| | HC N55E&S105A-LC S32A | 10 |
| 72C1B6A3 | HC WT-LC N31E | 4 |
| | HC WT-LC S32A | 5 |
| 120B7B2 | HC G57D&S104A-LC-N96E&N31E | 6 |
| | HC G57D&S104A-LC-S32A&G97A | 7 |

*The amino acid location (e.g., N55) is according to the amino acid residue number in the corresponding VH or VL amino acid sequence, not Kabat or Chothia.

| | | | SEQ |
|---|---|---|---|
| Antibody | Mutant | Sequence (mutation highlighted) | ID NO: |
| 4F11E2 | HC N55Q | EVQLVESGGGLVQPGGSLRLSCAASGFTFSTFGMHWVRQAPGKGLEWVSY | 196 |
| | | ITSGQSPIYFTDTVKGRFTISRDNAKNSLYLQMNSLRAEDTAVYYCARSS | |
| | | YYGNSMDYWGQGTLVTVSS | |
| | HC N55E | EVQLVESGGGLVQPGGSLRLSCAASGFTFSTFGMHWVRQAPGKGLEWVSY | 197 |
| | | ITSGESPIYFTDTVKGRFTISRDNAKNSLYLQMNSLRAEDTAVYYCARSS | |
| | | YYGNSMDYWGQGTLVTVSS | |
| | HC N55E & | EVQLVESGGGLVQPGGSLRLSCAASGFTFSTFGMHWVRQAPGKGLEWVSY | 198 |
| | N104Q | ITSGESPIYFTDTVKGRFTISRDNAKNSLYLQMNSLRAEDTAVYYCARSS | |
| | | YYGQSMDYWGQGTLVTVSS | |
| | HC N55E & | EVQLVESGGGLVQPGGSLRLSCAASGFTFSTFGMHWVRQAPGKGLEWVSY | 199 |
| | N104E | ITSGESPIYFTDTVKGRFTISRDNAKNSLYLQMNSLRAEDTAVYYCARSS | |
| | | YYGESMDYWGQGTLVTVSS | |
| | HC N55E & | EVQLVESGGGLVQPGGSLRLSCAASGFTFSTFGMHWVRQAPGKGLEWVSY | 200 |
| | S105A | ITSGESPIYFTDTVKGRFTISRDNAKNSLYLQMNSLRAEDTAVYYCARSS | |
| | | YYGNAMDYWGQGTLVTVSS | |
| | LC N31E | DIVMTQSPDSLAVSLGERATINCRSSQSLLESGNRKNYLTWYQQKPGQPP | 201 |
| | | KLLIYWASTRESGVPDRFSGSGSGTDFTLTISSLQAEDVAVYYCQNAYSY | |
| | | PFTFGGGTKLEIK | |
| | LC S32A | DIVMTQSPDSLAVSLGERATINCRSSQSLLNAGNRKNYLTWYQQKPGQPP | 202 |
| | | KLLIYWASTRESGVPDRFSGSGSGTDFTLTISSLQAEDVAVYYCQNAYSY | |
| | | PFTFGGGTKLEIK | |
| 72C1B6A3 | HC WT | QVQLVQSGAEVKKPGASVKVSCKASGYTFTTYPIEWVRQAPGQRLEWMGN | 181 |
| | | FHPYNDDTKYNEKFKGRVTITRDTSASTAYMELSSLRSEDTAVYYCARRA | |
| | | YGYPYAMDYWGQGTLVTVSS | |
| | LC S32A | DIVMTQSPDSLAVSLGERATINCKSSQSLLNAGNQKNYLTWYQQKPGQPP | 203 |
| | | KLLIYRASSRESGVPDRFSGSGSGTDFTLTISSLQAEDVAVYYCQNDYIY | |
| | | PYTFGGGTKLEIK | |
| | LC N31E | DIVMTQSPDSLAVSLGERATINCKSSQSLLESGNQKNYLTWYQQKPGQPP | 204 |
| | | KLLIYRASSRESGVPDRFSGSGSGTDFTLTISSLQAEDVAVYYCQNDYIY | |
| | | PYTFGGGTKLEIK | |
| 120B7B2 | HC G57D & | QVQLVQSGAEVKKPGASVKVSCKASGYTFTGYIIQWVRQAPGQRLEWMGF | 205 |
| | S104A | INPYNDDTKYNEQFKGRVTITRDTSASTAYMELSSLRSEDTAVYYCARAY | |
| | | FGNAFAYWGQGTLVTVSS | |
| | LC-S32A & | DIVMTQSPDSLAVSLGERATINCKSSQSLLNAGNQKNYLTWYQQKPGQPP | 206 |
| | G97A | KLLIYWASTRESGVPDRFSGSGSGTDFTLTISSLQAEDVAVYYCQNAYYF | |
| | | PFTFGGGTKLEIK | |
| | LC-N96E & | DIVMTQSPDSLAVSLGERATINCKSSQSLLESGNQKNYLTWYQQKPGQPP | 207 |
| | N31E | KLLIYWASTRESGVPDRFSGSGSGTDFTLTISSLQAEDVAVYYCQEGYYF | |
| | | PFTFGGGTKLEIK | |

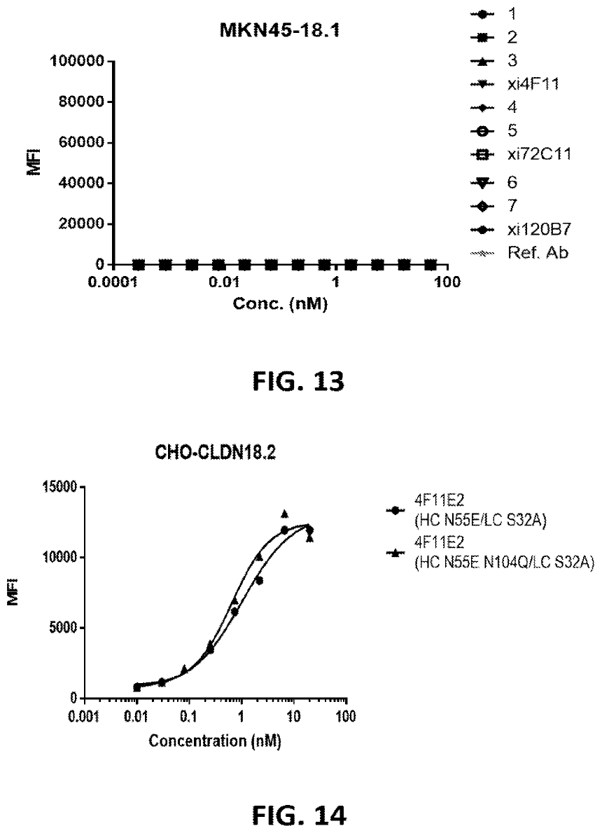

MKN45 cells that stably expressed human CLD18A2 or CLD18A1 were harvested from flasks. 100 μl of 1×10⁶ cells/ml of cells were incubated with primary mutated humanized antibodies indicated as FIG. 4 in 3-fold serial dilutions starting from 100 nM to 0.003 nM for 30 minutes on ice. After being washed with 200 μl of FACS buffer twice, cells were incubated with secondary antibody for 30 minutes on ice. Cells were washed with 200 μl of FACS buffer twice and transferred to BD Falcon 5 ml tube and analyzed by FACS. The results of the study showed that the indicated antibodies can bind to human CLD18A2 with high EC50, but not CLD18A1 (FIGS. 12 and 13).

To evalute the de-risked variants of 4F11E2d (HC N55E/LC S32A) and 4F11E2d (H N55E N104Q/LC S32A) antigen binding potency, the variants were tested in a cell-based binding assay. Serially diluted anti-CLDN18.2 antibodies, starting from 100 nM, were incubated with 10⁵ cells for 30 minutes on ice. After being washed with FACS buffer, cells were then incubated with APC labeled secondary antibody for another 30 minutes on ice. Cells bound with antibodies were analyzed by FACS. The variants showed potent binding to cell surface claudin 18.2 (FIG. 14).

### Example 12. Antibody-Dependent Cellular Cytotoxicity (ADCC) of PTM De-Risked Humanized Antibodies

Serial dilutions of claudin 18.2 PTM de-risked humanized antibodies or Ref. Ab were incubated for 6 hours of induction at 37° C. with engineered Jurkat effector cells (ADCC Bioassay Effector Cells), with or without ADCC Bioassay Target Cells (expressing claudin 18.2). Luciferase activity was quantified using Bio-Glo™ Reagent (Table 5). The results show that these humanized antibodies have very strong ADCC activities.

TABLE 5
ADCC

| No. | Tested antibody | EC50 (pM) |
|---|---|---|
| 1 | 4F11E2-HC N55Q-LC N31E | 238.1 |
| 2 | 4F11E2-HC N55Q-LC S32A | 413.9 |
| 3 | 4F11E2-HC N55E-LCS32A | 148.1 |
| 4 | 72C1B6A3-HC WT-LC N31E | 1651 |
| 5 | 72C1B6A3-HC WT- LCS32A | 190.5 |
| 6 | 120B7B2-HC G57D&104A-LC-N96E&N31E | 492.6 |
| 7 | 120B7B2-HC G57D&10 4A-LC-S32A&G97A | 113.9 |
| Ref. Ab | Ref. Ab | 158.3 |

### Example 13. Epitope Mapping

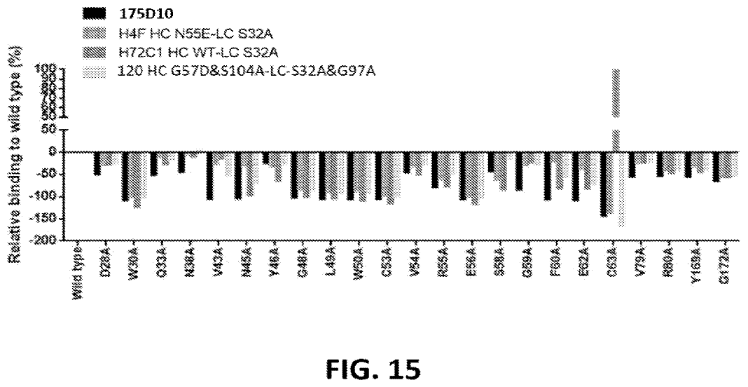

All amino acids of extracellular domain of claudin 18.2 were individually mutated to A. Each mutated or wildtype claudin 18.2 was transfected into Hek293 cells. The expression of claudin 18.2 was evaluated by indicated antibodies. The results are shown in FIG. 15 (only amino acid residues at which the mutation reduced bindings are shown).

As shown in FIG. 15, amino acids W30, N45, Y46, G48, L49, W50, C53, V54, R55, E56, S58, F60, E62, C63, R80, Y169, and G172 are involved in the binding of the three tested antibodies, 4F11E2 (H4F), 72C186A3 (H72C1) and 120B7B2 (120), or the reference antibody 175D10 (IMAB362). W30 appeared to form a cluster of residues at the first half of the first extracellular domain of the claudin 18.2 protein. N45, Y46, G48, L49, W50, C53, V54, R55, E56, S58, F60, E62 and C63 appeared to be a second cluster of residues within the same extracellular domain. Y169 and G172, on the other hand, are located at or close to the second extracellular domain.

The crystal structures of various claudin proteins have been resolved. As shown in FIG. 20 (adapted from Suzuki et al., Ann. N.Y. Acad. Sci., 1397:25-34), claudin proteins include four transmembrane segments, a short intracellular N-terminus, a large first extracellular loop (loop 1, or ECS1) that contains a consensus W-LW-C-C motif, a shorter second extracellular loop (loop 2, or ECS2), and an intracellular C-terminal tail. Loop 1 includes four β strands β1, β2, β3, and β4, and loops includes one β strand, B5.

The mutations of W30, L49 and W50 to alanine likely destabilized the conformation of loop 1. The mutation of C53 or C63 likely disrupted the disulfide bond between β3 and β4. R80 is likely important for maintaining the interaction between parallel claudin 18.2 molecules on the cell surface, or for stabilizing the conformation of loop 1. The remaining residues, including N45, Y46, G48, V54, R55, E56, S58, F60, and E62 (in the β3 to β4 loop), and Y169 and G172 (in β5), likely present the interface for binding to the antibodies tested here.

### Example 14. Comparison of Humanized 4F11E2, 72C1B6A3 and 120B7B2 Antibodies with Benchmark 175D10 Claudin 18.2 Antibody

### Cell Based Binding

To compare the humanized anti-claudin18.2 antibodies: 4F11E2 (HC N55E/LC S32A), 72C1B6A3 (HC WT/LC S32A) and 120B7B2 (HC G57D S104A/LC S32A G97A) to benchmark antibody 175D10 (IMAB362), this example determined the cell-based binding in human claudin 18.2 expressed cells. CHO-K1 cells that stably expressed human CLD18A2 were sorted for high expressor and low expressor based on the level of human CLDN18.2 expression. Serially diluted anti-CLDN18.2 antibodies, starting from 100 nM, were incubated with 10⁵ cells for 30 minutes on ice. After being washed with FACS buffer, cells were then incubated with APC labeled secondary antibody for another 30 minutes on ice. Cells bound with antibodies were analyzed by FACS.

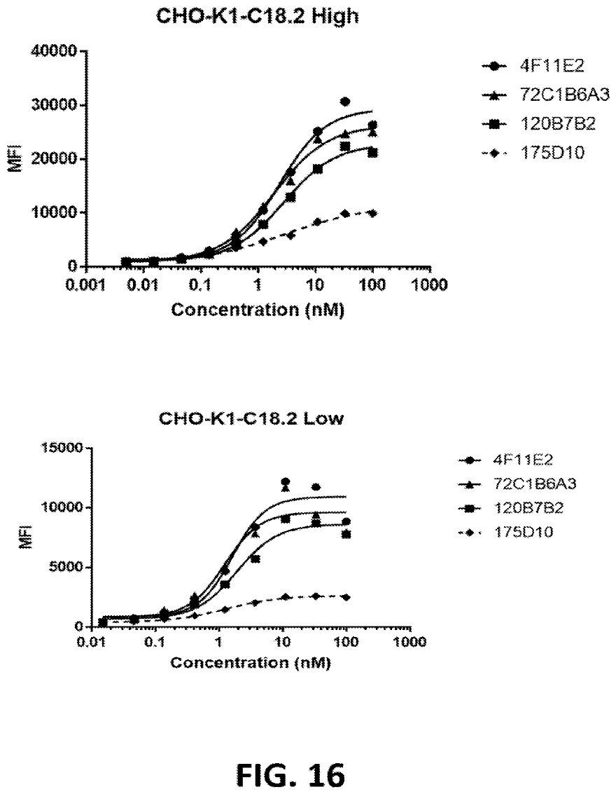

As shown in the FIG. 16, 4F11E2, 72C1B6A3 and 120B7B2 showed superior binding to 175D10 in both claudin 18.2 high and low CHO-K1 cells.

### ADCC Assay

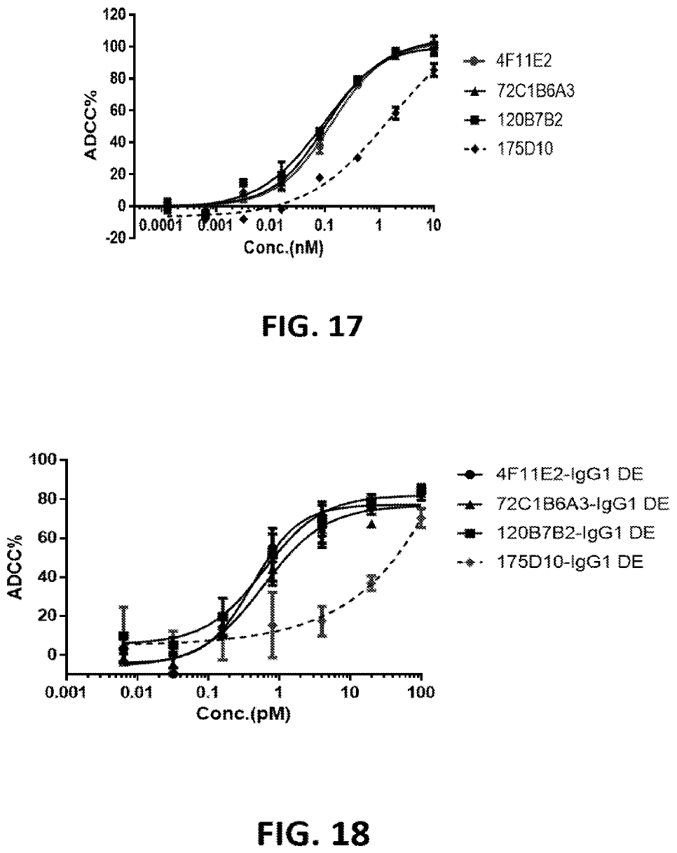

To further compare the ADCC effect of humanized anti-claudin18.2 antibodies: 4F11E2 (HC N55E/LC S32A), 72C1B6A3 (HC WT/LC S32A) and 120B7B2 (HC G57D S104A/LC S32A G97A) to benchmark antibody 175D10 (IMAB362), this example performed a cell based ADCC assay. Briefly, NK92 cells were cocultured with claudin18.2 overexpressed 293 cells in the presence of different dose anti-claudin 18.2 antibodies. As shown in FIG. 17, 4F11E2, 72C1B6A3 and 120B7B2 showed superior ADCC potency to the 175D10 antibody.

For certain therapeutic antibodies, enhanced ADCC may increase the therapeutic window to antibody-based target therapy. Enhanced ADCC may be achieved through engineering the Fc region such as with the S239D/1332E mutations. In a NK92 cell based ADCC assay, the 4H11E2, 72C1B6A3 and 120B7B2 antibodies with S239D/1332E mutations in the Fc region mediated stronger NK92-mediated cell killing of claudin 18.2 overexpressed 293 cells as compared to the control antibody 175D10 with the same S239D/1332E mutations (FIG. 18).

### Antibody-Dependent Cellular Phagocytosis (ADCP)

The effect of anti-CLDN 18.2 mAbs on the tumor cell phagocytosis by macrophages was evaluated in an in vitro assay in which CLDN18.2 positive NUG-C4 cells were co-cultured with human differentiated macrophages in the presence of different concentration of anti-CLDN18.2 mAbs. In short, CD14+ monocytes were purified from human peripheral blood mononuclear cells (PBMCs) and in vitro differentiated into mature macrophages for 6 days. The monocyte derived macrophages (MDMs) were collected and re-plated in 24-well dishes overnight as effector cells. NUG-C4 expressing CLDN 18.2-eGFP as target cells were added to MDMs at a ratio of 5 tumor cells per phagocyte in the presence of different concentrations of anti-CLDN18.2 mAbs. After 3 hours' incubation, non-phagocytosed target cells were washed away with PBS and the remaining phagocytes were collected and stained with macrophage marker CD14 followed by flow cytometry analysis. Phagocytosis index was calculated by quantitating the percent of GFP+ cells in CD14+ cells, normalized to that of IgG control.

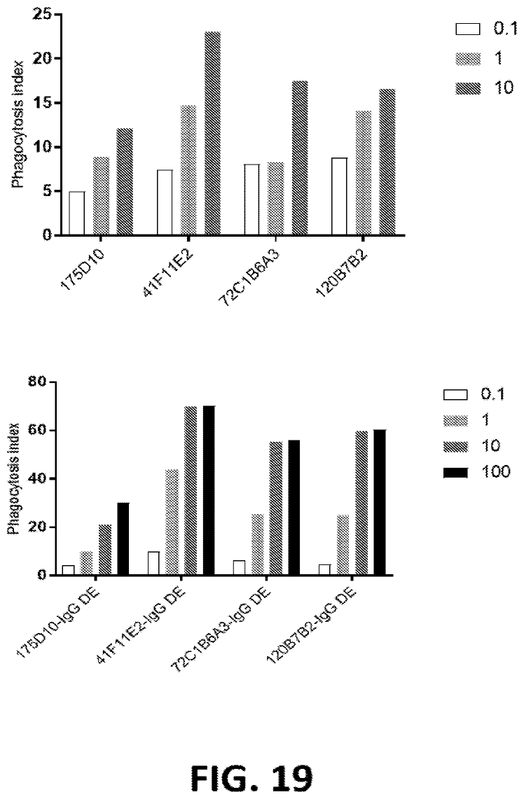

As shown in FIG. 19, all C18.2 mAbs significantly enhanced the phagocytosis of NUG-C4 cells in a concentration-dependent manner. In both wildtype IgG1 and S239D/1332E mutated IgG1 formats, 4H11E2, 72C1B6A3 and 120B7B2 antibodies showed stronger ADCP effect than the reference antibody 175D10.

Taken together, this example demonstrates that the newly developed 4F11E2, 72C1B6A3 and 120B7B2 antibodies had stronger cell-based binding and ADCC/ADCP potency than the reference antibody 175D10. It is contemplated that the improved properties of these new antibodies can be attributed to the higher binding specificity of these antibodies as compared to that of the reference antibody 175D10. For instance, 175D10's interaction with claudin 18.2 is strong across the spectrum in FIG. 15, which includes strong binding to D28, Q33, N38 and V43, and then G59 and V79. The new antibodies, 4F11E2, 72C1B6A3 and 120B7B2, by contrast, have higher specificity to W30 within the first half of the first extracellular domain, and higher specificity to G48 through E56 within the second half of the first extracellular domain. The new antibodies also have slightly stronger binding to Y46 which is also in the second half. Their binding to D28, Q33, N38, V43, G59 and V79 is considerably weaker, which likely contributed to the improved ADCC and ADCP of the new antibodies.

### Example 15: PHAb Conjugation to Claudin 18.2 Antibodies

Internalization of CLDN18.2 bound anti-Claudin 18.2 antibodies was determined using pHAbReactive Dyes based internalization assay. pHAb Dyes are pH sensor dyes that have very low fluorescence at pH>7 and a dramatic increase in fluorescence as the pH of the solution becomes acidic. pHAb Dyes have excitation maxima (Ex) at 532 nm and emission maxima (Em) at 560 nm. pHAb Dye-conjugated antibodies can be used for monitoring receptor-mediated antibody internalization. When an antibody-pHAb Dye conjugate binds to its receptor on the cell membrane, it exhibits minimal fluorescence. However, upon receptor-mediated internalization, antibody-pHAb Dye conjugates traffic to the endosome and lysosomal vesicles where pH is acidic, causing the pHAb Dye to fluoresce. This fluorescence can be detected using various techniques, including cell imaging, flow cytometry and fluorescent plate-based readers with appropriate filters.

### Experimental Protocol:

### A. Antibodies Production

27 chimeric antibodies, 3 humanized antibodies and a control IgG1 were produced by transiently transfecting ExpiCHO cells and purified by Protein A affinity chromatography.

B. On-Bead Antibody Conjugation Using pHAb Thiol Reactive Dye

1. Gently shake or use an end-over-end mixer to uniformly resuspend the AmMag® Protein A Beads (LC00695). Keep the suspension uniform when making aliquots of beads.

2. Add 50 μl of bead slurry to a 1.5 ml microcentrifuge tube. Place the tube on the magnetic stand for 10 seconds.

3. Remove and discard the storage buffer.

4. Add 250 μl of PBS (pH7.4). Mix and place tube on the magnetic stand for 10 seconds. Remove and discard the buffer.

5. Add 1.0 ml of sample containing 100 μg of antibody to the beads.

6. Mix the sample for 60 minutes at room temperature. Keep the beads in suspension by mixing continuously.

7. Place the tube in the magnetic stand for 10 seconds. Remove the supernatant.

8. Add 250 μl of thiol conjugation buffer (10 mM phosphate buffer containing 1 mM EDTA, pH 7.0) and mix. Place the tube in the magnetic stand for 10 seconds. Remove and discard the buffer. Repeat this step for a total of two washes.

9. Add 100 μl of thiol conjugation buffer.

10. Add DTT to a final concentration of 2.5 mM.

11. Mix sample for 60 minutes at room temperature. Keep the beads in suspension by mixing continuously.

12. Place the tube in the magnetic stand for 10 seconds and discard the buffer.

13. Add 250 μl of thiol conjugation buffer and mix. Place the tube in the magnetic stand for 10 seconds. Remove and discard the buffer. Repeat this step for a total of two washes.

14. Add 100 μl of thiol conjugation buffer.

15. Quickly centrifuge the pHAb Thiol Reactive Dye (G9835) (i.e., 14,000×g in a tabletop centrifuge for 5-10 seconds) and dissolve at 10 mg/ml by adding 25 μl of 1:1 DMSO-water mix to 0.25 mg of dye. Mix by vortexing. It may take 1-3 minutes for the dye to dissolve completely. Make this solution just before use.

16. Add 1.2 μl of pHAb Thiol Reactive Dye for 100 μg of antibody to make a 20 molar excess of dye.

17. Mix for 60 minutes. Keep the beads in suspension by mixing continuously.

18. Place the tube in the magnetic stand for 10 seconds. Remove and discard the supernatant. 19. Add 250 μl of thiol conjugation buffer and mix. Place in the magnetic stand for 10 seconds. Remove and discard the bind/wash buffer (PBS.pH7.4).

20. Repeat Step 19 for a total of two washes.

21. Add 100 μl of elution buffer (0.1M Glycine, pH3.0) to the beads.

22. Mix for 5 minutes at room temperature.

23. Place the tube in the magnetic stand for 10 seconds. Remove eluted sample and transfer to a new microcentrifuge tube containing 5 μl of neutralization buffer (1M Tris-HCl, pH9.0).

The antibody concentration and dye-to-antibody ratio (DAR) for the tested antibodies are shown in Table 6.

TABLE 6

| Dye-to-antibody ratio (DAR) | |
|---|---|
| Clone | DAR |
| 64G11B4 | 2.5 |
| 65G8B8 | 4.2 |
| 56E8F10F4 | 3.2 |
| 44F6B11 | 3.0 |
| 15C2B7 | 3.0 |
| 20F1E10 | 2.7 |
| 58G2C2 | 3.1 |
| 101C4F12 | 2.4 |
| 103A10B2 | 3.5 |
| 78E8G9G6 | 2.3 |
| 10G7G11 | 3.1 |
| 12F1F4 | 2.4 |
| 78C10B6G4 | 2.9 |
| 119G11D9 | 3.2 |
| 113G12E5E6 | 2.9 |
| 116A8B7 | 2.6 |
| 105F7G12 | 2.8 |
| 84E9E12 | 2.8 |
| 103F4D4 | 2.1 |
| 110C12B6 | 2.8 |
| 85H12E8 | 3.8 |
| 103H2B4 | 2.7 |
| 103F6D3 | 3.1 |
| 113E12F7 | 3.9 |
| 111B12D11 | 2.7 |
| 111E7E2 | 3.5 |
| 100F4G12 | 3.0 |
| 4F11E2 HC N55E-LC S32A | 3.0 |
| (BG2001-C) | |
| 72C1B6A3 HC WT-LC | 2.6 |
| S32A(BG2001-D) | |
| 120B7B2 HCG57D&S104A-LC | 3.0 |
| S32A&G97A (BG2001-E) | |
| IMAB362 (Ref. Ab) | 3.3 |
| IgG (Control) | 2.9 |

### Example 16: Screen for Claudin 18.2 Antibodies' Internalization

Stably transfected human CLDN18.2 MKN45 cells were harvested with 0.05% Trypsin/EDTA (Gibco, 25300-054) and plated in a 96-well black plate (Thermo Scientific #165305) at the density of 20 K per 90 μl per well. Plates were incubated for 20-24 h before treatment with pHAb labeled antibodies.

For internalization, pHAb conjugated claudin 18.2 antibodies were added to the cells at two concentrations (20 nM and 100 nM) and mixed gently for 1-2 min on a plate mixer and then incubated overnight to allow internalization (internalization can be detected in a few hours). Plates were read on a fluorescent plate reader at Ex/Em: 532 nm/560 nm on a Tecan Infinity M1000 Pro. To achieve higher sensitivity, media was replaced by PBS before reading the plate.

The results normalized by DAR are shown in Table 7. The internalization efficiencies of the tested antibodies were greater than the reference antibody IMAB362.

TABLE 7
Internalization Results

| | Fluorescence | |
|---|---|---|
| Clone | 20 nM | 100 nM |
| 64G11B4 | 24030 | 33742 |
| 65G8B8 | 15101 | 20466 |
| 56E8F10F4 | 19865 | 28672 |
| 44F6B11 | 18438 | 26089 |
| 15C2B7 | 19400 | 33174 |
| 20F1E10 | 21146 | 45093 |
| 58G2C2 | 18645 | 33059 |
| 101C4F12 | 15179 | 44394 |
| 103A10B2 | 11998 | 31328 |
| 78E8G9G6 | 26815 | 41148 |
| 10G7G11 | 9603 | 35549 |
| 12F1F4 | 29457 | 43854 |
| 78C10B6G4 | 16190 | 29146 |
| 119G11D9 | 21438 | 34049 |
| 113G12E5E6 | 11003 | 28896 |
| 116A8B7 | 21356 | 41720 |
| 105F7G12 | 14848 | 43122 |
| 84E9E12 | 18710 | 35080 |
| 103F4D4 | 31176 | 49098 |
| 110C12B6 | 19335 | 40139 |
| 85H12E8 | 19710 | 29070 |
| 103H2B4 | 23459 | 35834 |
| 103F6D3 | 23023 | 41266 |
| 113E12F7 | 13181 | 29861 |
| 111B12D11 | 15926 | 37384 |
| 111E7E2 | 12335 | 28040 |
| 100F4G12 | 17093 | 33537 |
| 4F11E2 HC N55E-LC S32A | 19008 | 28208 |
| (BG2001-C) | | |
| 72C1B6A3 HC WT-LC S32A | 26203 | 36535 |
| (BG2001-D) | | |
| 120B7B2 HC G57D&S104A-LC | 19204 | 31321 |
| S32A&G97A (BG2001-E) | | |
| IMAB362 (Ref. Ab) | 7951 | 18907 |
| IgG (Control) | 854 | 2705 |

### Example 17: EC50 of Internalization of Chimeric Claudin 18.2 Antibodies on CHO-Claudin 18.2 Cells

Stably transfected human CLDN18.2 CHO cells were harvested with 0.05% Trypsin/EDTA (Gibco, 25300-054) and plated in a 96-well black plate (Thermo Scientific #165305) at the density of 10 K per 90 μl per well. Plates were incubated for 20-24 h before treatment with pHAb labeled antibodies.

For internalization, pHAb conjugated chimeric claudin 18.2 antibodies were added to the cells at different concentrations (100 nM, 30 nM, 10 nM, 3 nM, 1 nM, 0.3 nM, 0.1 nM, 0.03 nM and 0.01 nM) and mixed gently for 1-2 min on a plate mixer and then incubated overnight to allow internalization (internalization can be detected in a few hours). Plates were read on a fluorescent plate reader at Ex/Em: 532 nm/560 nm on a Tecan Infinity M1000 Pro. To achieve higher sensitivity, media was replaced by PBS before reading the plate.

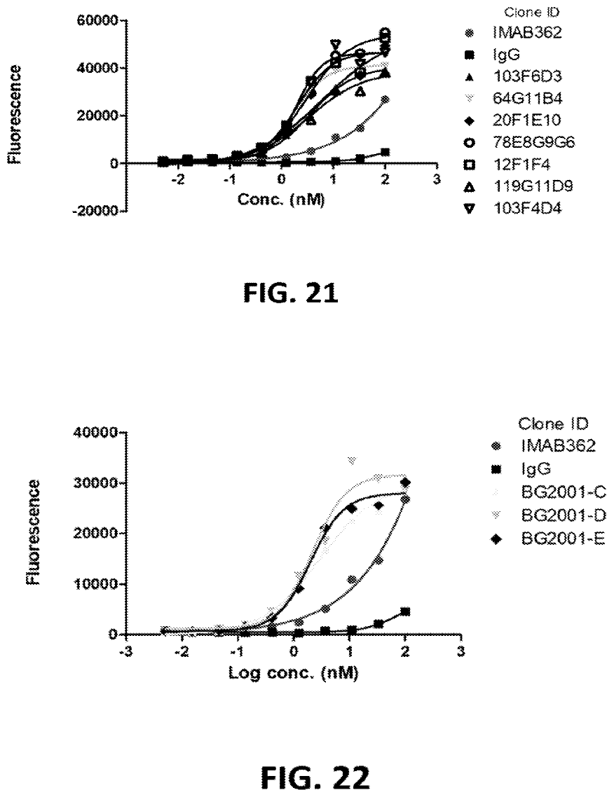

The results normalized with DAR are shown in the FIG. 21. Again, the internalization efficiencies of the tested antibodies were greater than the reference antibody IMAB362.

### Example 18: EC50 of Internalization of Humanized Claudin 18.2 Antibodies on CHO-Claudin 18.2 Cells

Stably transfected human CLDN18.2 CHO cells were harvested with 0.05% Trypsin/EDTA (Gibco, 25300-054) and plated in a 96-well black plate (Thermo Scientific #165305) at the density of 10 K per 90 μl per well. Plates were incubated for 20-24 h before treatment with pHAb labeled antibodies.

For internalization, pHAb conjugated humanized claudin 18.2 antibodies were added to the cells at different concentrations (100 nM, 30 nM, 10 nM, 3 nM, 1 nM, 0.3 nM, 0.1 nM, 0.03 nM and 0.01 nM) and mixed gently for 1-2 min on a plate mixer and then incubated overnight to allow internalization (internalization can be detected in a few hours). Plates were read on a fluorescent plate reader at Ex/Em: 532 nm/560 nm on a Tecan Infinity M1000 Pro. To achieve higher sensitivity, media was replaced by PBS before reading the plate.

The results normalized with DAR are shown in the FIG. 22, which shows that the internalization efficiencies of the tested antibodies were greater than the reference antibody IMAB362.

### Example 19: EC50 of Internalization of Humanized Claudin 18.2 Antibodies on MKN45-Caludin 18.2 Cells

Stably transfected human CLDN18.2 MKN45 cells were harvested with 0.05% Trypsin/EDTA (Gibco, 25300-054) and plated in a 96-well black plate (Thermo Scientific #165305) at the density of 10 K per 90 μl per well. Plates were incubated for 20-24 h before treatment with pHAb labeled antibodies.

For internalization, pHAb conjugated humanized claudin 18.2 antibodies were added to the cells at different concentrations (100 nM, 30 nM, 10 nM, 3 nM, 1 nM, 0.3 nM, 0.1 nM, 0.03 nM and 0.01 nM) and mixed gently for 1-2 min on a plate mixer and then incubated overnight to allow internalization (internalization can be detected in a few hours). Plates were read on a fluorescent plate reader at Ex/Em: 532 nm/560 nm on a Tecan Infinity M1000 Pro. To achieve higher sensitivity, media was replaced by PBS before reading the plate.

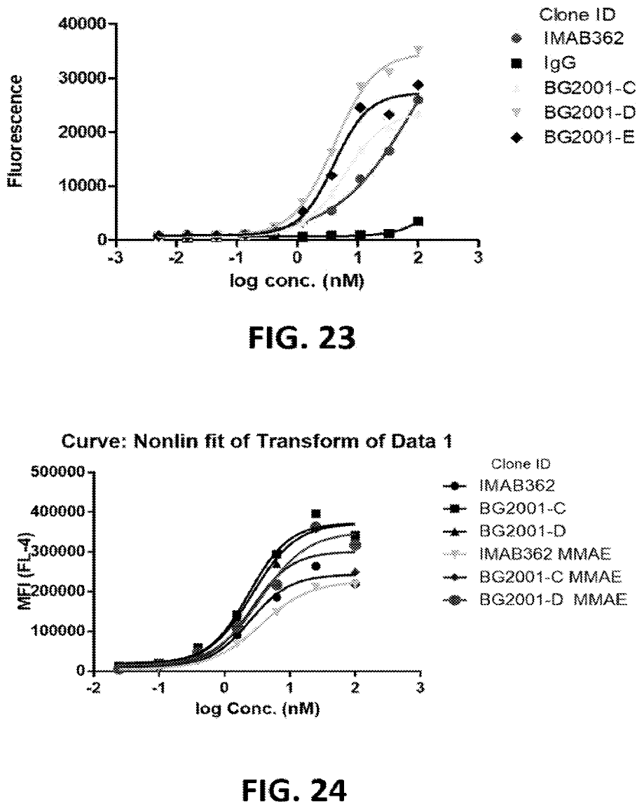

The results normalized with DAR are shown in the FIG. 23, which shows that the internalization efficiencies of the tested antibodies were greater than the reference antibody IMAB362.

### Example 20: Antibody Drug Conjugates

Each antibody was mixed with approximately three-fold TCEP and was stirred for 2 h at 37° C. The reaction system was quickly dropped over eight-fold for VC-MMAE and was incubated for 1 h on ice, and 20-fold excess of cysteine was added over the drug linker to extinguish the reaction. Finally, the ADC product was purified by elution through Sephadex G-25 equilibrated in PBS and concentrated by centrifugal ultrafiltration. The conjugate was filtered through a 0.2 μm filter under sterile conditions and stored at −80° C. for analysis and testing. Drug antibody ratio was analyzed by UV spectrometry, the monomer content by SEC-HPLC and free drug content by RP-HPLC. The (DAR) of vcMMAE conjugated antibodies are shown in Table 8.

TABLE 8

| DAR of vcMMAE conjugated antibodies | |
|---|---|
| Antibodies | DAR |
| 4F11E2 HC N55E-LC S32A | 3.76 |
| 72C1B6A3 HC WT-LC S32A | 3.93 |
| IMAB362 (Ref. Ab) | 4.00 |
| IgG1 (Control) | 3.81 |

### Example 21: Relative Binding Affinities and Specificities of Anti-CLDN18.2 Naked Antibodies and Antibody Drug Conjugates

This example determined the relative binding affinities and specificities of anti-CLDN18.2 naked antibodies and antibody drug conjugates by flow cytometry using CLDN18.2 positive and negative cell lines.

Cells from an exponentially growing culture were harvested with 0.05% Trypsin/EDTA (Gibco, 25300-054), and counted using a Neubauer counting chamber. Cells were centrifuged 5 min at 1,500 rpm (468×g), the supernatant was discarded and cells were resuspended in FACS buffer (PBS containing 2% FCS (Gibco, 10270-106) for analysis with toxin-conjugated antibodies, PBS containing 2% FCS and 2 mM EDTA for screening of CLDN18.2 reactive naked antibodies) at 2×10⁶ cells/ml. 100 μl of the cell suspension per well (correspond to 2×10⁵ cells/well) were transferred to a round bottom 96-well microtiter plate. After centrifugation for 1 min at 1500 rpm, supernatant was discarded and cells were resuspended in FACS buffer containing toxin-conjugated or naked antibodies at appropriate concentrations (up to 20 μg/ml for relative affinity measurement or 50 μg/ml for expression control) and incubated for 30-45 min at 4° C. (Table 8). The cells were centrifuged for 1 min at 1500 rpm and the supernatant was discarded. After the cells were washed three times with FACS buffer, they were resuspended in FACS buffer containing APC-conjugated anti-human IgG (Jackson Immuno Research, 109-136-170) or APC-conjugated goat-anti-mouse IgG (Jackson Immuno Research, 115-136-146) or Protein L-FITC (1 μg/ml, analysis of chim mAB294) and incubated for 30 min at 4° C. (Table 3). After incubation, 100 μl FACS buffer were added to each sample, the cells were centrifuged for 1 min at 1500 rpm and the supernatant was discarded. The washing step with FACS buffer was repeated twice. Finally, the cells were resuspended in 100 μl FACS buffer and binding was determined using a BD FACS Array Bioanalyzer.

It should be noted that toxin-conjugated and naked antibodies were applied at equal concentrations. The results are shown in FIG. 24.

### Example 22: Cell Toxicity of Claudin 18.2 Humanized Antibodies with MMAE is More Potent than IMAB362 with MMAE in DAN-G, NUGC or SCG-7901 Transfectants

Cells overexpressing human claudin 18.2 (DAN-G, NUGC or SCG-7901 transfectants) were harvested with 0.05% Trypsin/EDTA (Gibco, 25300-054), resuspended in cell culture medium and 50 μl of the cell suspension with the corresponding amount of cells were seeded per well in 96-well cell culture plates. After 24 h, toxin conjugated IMAB362 or control antibodies diluted in 50 μl medium at appropriate concentrations were added and cells were cultured for another 72 h. The effect of Claudin 18.2 humanized antibodies with MMAE on cell viability was determined using a CellTiter-Glo® Luminescent Cell Viability Assay (G7572).

CellTiter-Glo® Luminescent Cell Viability Assay (G7572) protocol used was as follows:

1. Prepare opaque-walled multiwell plates with mammalian cells in culture medium, 100 μl per well for 96-well plates or 25 μl per well for 384-well plates. Multiwell plates must be compatible with the luminometer used.

2. Prepare control wells containing medium without cells to obtain a value for background luminescence.

3. Add the test compound to experimental wells, and incubate according to culture protocol. 4. Equilibrate the plate and its contents at room temperature for approximately 30 minutes

5. Add a volume of CellTiter-Glo® Reagent equal to the volume of cell culture medium present in each well (e.g., add 100 μl of reagent to 100 μl of medium containing cells for a 96-well plate, or add 25 μl of reagent to 25 μl of medium containing cells for a 384-well plate).

6. Mix contents for 2 minutes on an orbital shaker to induce cell lysis.

7. Allow the plate to incubate at room temperature for 10 minutes to stabilize luminescent signal. Note: Uneven luminescent signal within standard plates can be caused by temperature gradients, uneven seeding of cells or edge effects in multiwell plates.

8. Record luminescence.

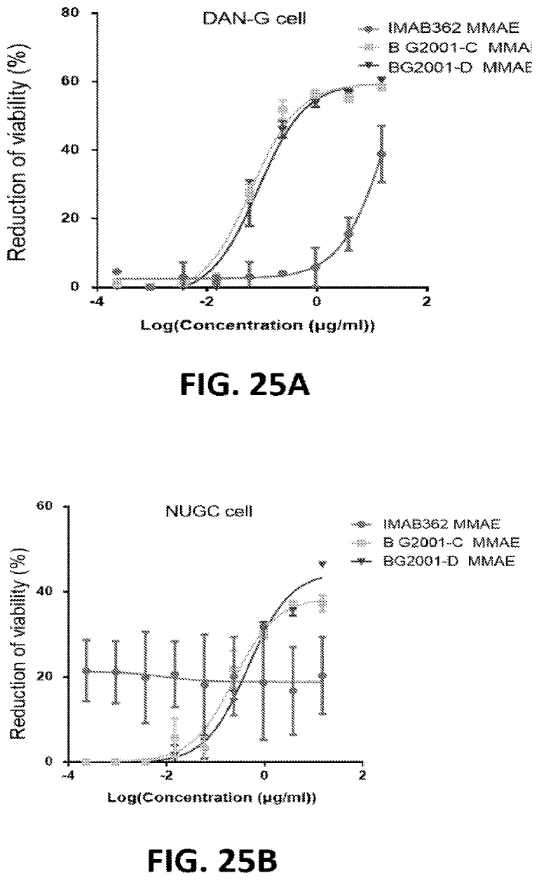

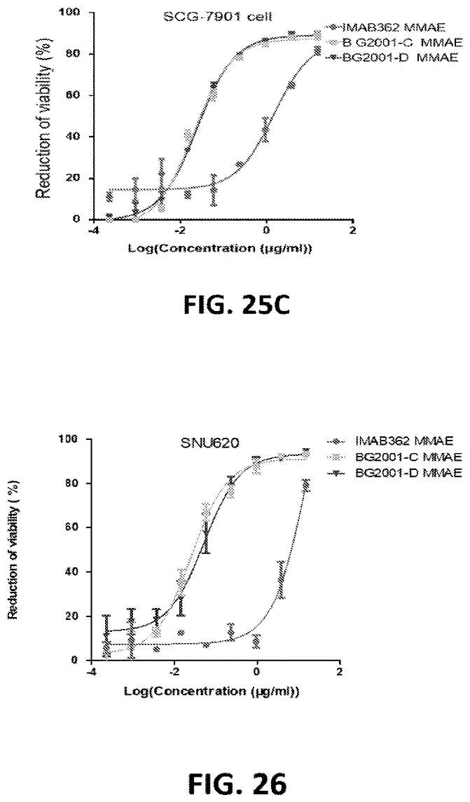

The testing results are shown in FIG. 25A-C. BG2001-C and BG2001-D both, when conjugated with MMAE, had greatly increased cytotoxicity as compared to the reference IMAB362-NMAE conjugate, in all tested cells. These results therefore demonstrate the improved ability of the presently disclosed antibodies in internalizing conjugated drugs.

### Example 23: Cell Toxicity of Claudin 18.2 Humanized Antibodies with MMAE is More Potent than IMAB362 with MMAE in SNU620 Endogenously Expressing Human Claudin 18.2

SNU620 cells were resuspended in cell culture medium and 50 μl of the cell suspension with the corresponding amount of cells were seeded per well in 96-well cell culture plates. After 24 h, toxin conjugated antibodies, including reference antibody IMAB362, diluted in 50 μl medium at appropriate concentrations were added and cells were cultured for another 72 h. The effect of Claudin 18.2 humanized antibodies with MMAE on cell viability was determined using a CellTiter-Glo® Luminescent Cell Viability Assay (G7572). As shown in FIG. 26, BG2001-C and BG2001-D both were greatly more effective in delivering the conjugated MMAE into the SNU620 cells as compared to the reference antibody IMAB362, a lead anti-claudin 18.2 antibody under clinical development.

### Example 24: In Vivo Efficacy of the Antibody Drug Conjugates

This example tested the efficacy of one of the antibody-drug conjugates (ADC), as compared to the antibody alone (mAb), in reducing tumor growth in nude mice transplanted with human tumor cells.

0.1 mL (5×10⁵ cells) of human patient derived cells (mixed with Matrigel 1:1) were subcutaneously inoculated on the right back of each mouse. When the average tumor volume reached 60˜80 mm³, 30 mice were selected for treatment experiments.

Eighteen days following inoculation, 5 mice having a tumor size in the range of 330-520 mm³ were selected for each treatment (1 mg/kg, 3 mg/mk, 10 mg/kg or 20 mg/kg ADC, QW) for three weeks. For comparison, antibody (mAb) only treatment was at 10 mg/kg (BIW).

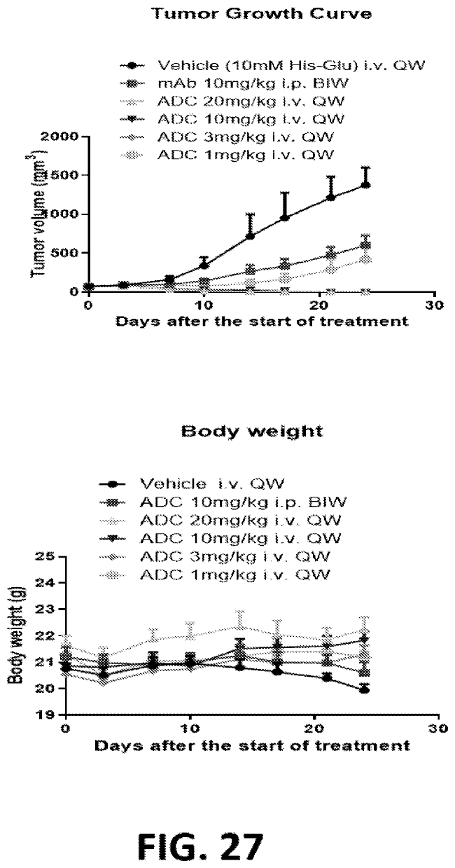

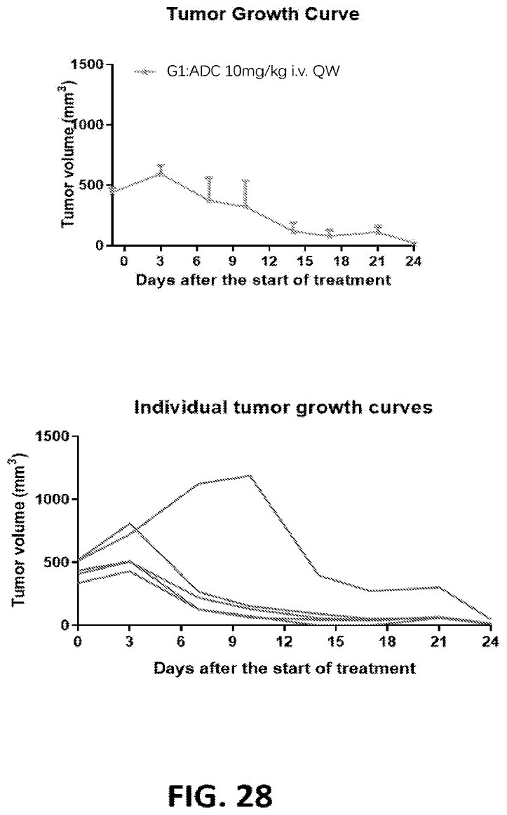

The results are presented in FIG. 27. ADC at both 10 mg/kg and 20 mg/kg completely inhibited tumor growth, without reducing the animals' body weights. FIG. 28 shows the average and individual tumor reduction effect in each animal. The tumor reduction effect of the ADC, therefore, is considerably greater than antibody alone.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody-drug conjugate, comprising a drug moiety covalently attached to an antibody or fragment thereof having binding specificity to a wild-type human claudin 18.2 protein, wherein the antibody or the fragment thereof comprises a light chain variable region comprising light chain complementarity determining regions CDRL1, CDRL2, and CDRL3 and a heavy chain variable region comprising heavy chain complementarity determining regions CDRH1, CDRH2, and CDRH3, wherein
the CDRL1 comprises the amino acid sequence of SEQ ID NO:304,
the CDRL2 comprises the amino acid sequence of SEQ ID NO:229,
the CDRL3 comprises the amino acid sequence of SEQ ID NO:8,
the CDRH1 comprises the amino acid sequence of SEQ ID NO:242,
the CDRH2 comprises the amino acid sequence of SEQ ID NO:263, and
the CDRH3 comprises the amino acid sequence of SEQ ID NO:289; and
wherein the drug moiety comprises monomethyl auristatin E (MMAE).

2. The antibody-drug conjugate of claim 1, wherein the antibody or the fragment thereof comprises a light chain variable region comprising an amino acid sequence of SEQ ID NO: 203 and a heavy chain variable region comprising an amino acid sequence of SEQ ID NO: 181.

3. The antibody-drug conjugate of claim 1, wherein the drug moiety is attached to the antibody or fragment thereof through a linker.

4. The antibody-drug conjugate of claim 3, wherein the linker is hydrolyzable under acidic conditions.

5. The antibody-drug conjugate of claim 1, wherein the antibody or fragment thereof is humanized.

6. The antibody-drug conjugate of claim 1, wherein the antibody or fragment thereof comprises a heavy chain constant region.

7. The antibody-drug conjugate of claim 6, wherein the heavy chain constant region has domains derived from an IgG₁ molecule.

8. The antibody-drug conjugate of claim 6, wherein the heavy chain constant region comprises S239D/1332E mutations according to EU numbering.

9. The antibody-drug conjugate of claim 1, wherein the antibody or fragment thereof comprises a light chain constant region.

10. The antibody-drug conjugate of claim 9, wherein the light chain constant region comprises a constant kappa domain or a constant lambda domain.

11. The antibody-drug conjugate of claim 3, wherein the linker comprises valine-citrulline (Val-Cit), phenylalanine-lysine (Phe-Lys), maleimidocapronic-valine-citrulline-p-aminobenzyloxycarbonyl (mc-Val-Cit-PABA), sulfosuccinimidyl-4-[N-maleimidomethyl]cyclohexane-1-carboxylate (smcc), or maleimidocaproyl (mc).

12. The antibody-drug conjugate of claim 1, wherein a ratio of the drug moiety to the antibody or fragment thereof is from 1:1 to 20:1.

13. The antibody-drug conjugate of claim 1, wherein a ratio of the drug moiety to the antibody or fragment thereof is from 2:1 to 6:1.

14. The antibody-drug conjugate of claim 1, wherein a ratio of the drug moiety to the antibody or fragment thereof is about 2:1, 2.5:1, 3:1, 3.5:1, 4:1, 4.5:1, or 5:1.

15. An antibody-drug conjugate, comprising a drug moiety covalently attached to a humanized antibody having binding specificity to a wild-type human claudin 18.2 protein, wherein the antibody comprises:
(1) a light chain variable region comprising an amino acid sequence of SEQ ID NO: 203 and a heavy chain variable region comprising an amino acid sequence of SEQ ID NO: 181,
(2) a heavy chain constant region has domains derived from an IgG₁ molecule and S239D/1332E mutations according to EU numbering,
(3) a light chain constant region that comprises a constant kappa domain or a constant lambda domain;
wherein the drug moiety comprises monomethyl auristatin E (MMAE);
wherein the drug moiety is attached to the antibody through a linker comprising maleimidocapronic-valine-citrulline-p-aminobenzyloxycarbonyl (mc-Val-Cit-PABA); and
wherein a ratio of the drug moiety to the antibody or fragment thereof is about 2:1, 2.5:1, 3:1, 3.5:1, 4:1, 4.5:1, or 5:1.

16. A pharmaceutical composition comprising:
(i) an antibody-drug conjugate that comprises a drug moiety covalently attached to an antibody or fragment thereof having binding specificity to a wild-type human claudin 18.2 protein, wherein the antibody or the fragment thereof comprises a light chain variable region comprising light chain complementarity determining regions CDRL1, CDRL2, and CDRL3 and a heavy chain variable region comprising heavy chain complementarity determining regions CDRH1, CDRH2, and CDRH3, wherein
the CDRL1 comprises the amino acid sequence of SEQ ID NO:304,
the CDRL2 comprises the amino acid sequence of SEQ ID NO:229,
the CDRL3 comprises the amino acid sequence of SEQ ID NO:8,
the CDRH1 comprises the amino acid sequence of SEQ ID NO:242,
the CDRH2 comprises the amino acid sequence of SEQ ID NO:263, and
the CDRH3 comprises the amino acid sequence of SEQ ID NO:289; and
the drug moiety comprises monomethyl auristatin E (MMAE); and
(ii) a pharmaceutically acceptable carrier.

17. The pharmaceutical composition of claim 16, wherein the antibody-drug conjugate comprises a drug moiety covalently attached to a humanized antibody having binding specificity to a wild-type human claudin 18.2 protein, wherein the antibody comprises:
(1) a light chain variable region comprising an amino acid sequence of SEQ ID NO: 203 and a heavy chain variable region comprising an amino acid sequence of SEQ ID NO:181,
(2) a heavy chain constant region has domains derived from an IgG₁ molecule and S239D/1332E mutations according to EU numbering,
(3) a light chain constant region that comprises a constant kappa domain or a constant lambda domain;
wherein the drug moiety comprises monomethyl auristatin E (MMAE);
wherein the drug moiety is attached to the antibody through a linker comprising maleimidocapronic-valine-citrulline-p-aminobenzyloxycarbonyl (mc-Val-Cit-PABA); and
wherein a ratio of the drug moiety to the antibody or fragment thereof is about 2:1, 2.5:1, 3:1, 3.5:1, 4:1, 4.5:1, or 5:1.
